(19) United States
(12) Patent Application Publication
(10) Pub. No.: US 2012/0225033 A1
(43) Pub. Date: Sep. 6, 2012

(54) Biodegradable Drug Delivery Composition

(75) Inventors: William W. van Osdol, Mountain View, CA (US); Su il Yum, Los Altos, CA (US); Felix Theeuwes, Los Altos Hills, CA (US); Michael Sekar, Palo Alto, CA (US); John Gibson, Springville, AL (US); Keith Branham, Pelham, AL (US); Huey-Ching Su, San Jose, CA (US)

(73) Assignee: Durect Corporation

(21) Appl. No.: 13/304,174

(22) Filed: Nov. 23, 2011

Related U.S. Application Data
(60) Provisional application No. 61/417,126, filed on Nov. 24, 2010.
(60) Provisional application No. 61/563,469, filed on Nov. 23, 2011.

(51) Int. Cl. A61K 38/27; A61K 38/16; A61K 38/26; A61K 38/21; A61K 31/7105

(52) U.S. Cl. 424/85.7; 514/11.4; 514/44 R; 514/11.7; 514/21.3

(57) ABSTRACT

The present disclosure provides a biodegradable drug delivery composition including a vehicle and an insoluble component comprising beneficial agent dispersed in the vehicle. Typically, the composition is not an emulsion, but has a low viscosity and further provides for minimized initial burst and sustained release of the beneficial agent over time. Also provided, are kits including the biodegradable drug delivery composition or components thereof, as well as methods of making and using the biodegradable drug delivery composition.

### CROSS-REFERENCE TO RELATED APPLICATIONS

The present application claims the benefit of and expressly incorporates by reference herein the entire disclosure of U.S. Provisional Application No. 61/417,126, filed Nov. 24, 2010; and U.S. Provisional Application entitled “Radiation-Sterilized Biodegradable Drug Delivery Composition,” Attorney Docket No. DURE-079PRV, filed on Nov. 23, 2011.

### BACKGROUND

A variety of compositions designed for the delivery of beneficial agent, such as depot compositions, are available which utilize various combinations of polymers, solvents and other components. However, many of these compositions require multiple components and/or preparation steps which serve to complicate the formulation process. In addition, various additives may be required in order to provide a composition suited to the desired mode of administration or to provide the desired release kinetics. For example, currently available formulations designed to provide extended release of beneficial agents often rely on high-viscosity vehicles which have poor syringeability and injectability and are therefore unsuitable for use with narrow gauge needles or needless injectors. Alternatively, existing low-viscosity formulations which may be suitable for injection often lack desired release kinetics, showing significant initial burst, followed by an exponentially declining release profile. The present disclosure addresses these issues and provides related advantages.

### SUMMARY OF THE INVENTION

The present disclosure provides biodegradable drug delivery compositions including a vehicle, e.g., a single phase vehicle, and an insoluble component comprising a beneficial agent in the vehicle. In some embodiments, the composition is not an emulsion, but has a low viscosity which can provide good injectability and syringeability and further provides for sustained release of the beneficial agent over time, and minimized initial burst. Also provided, are kits including the biodegradable drug delivery composition or components thereof, as well as methods of making and using the biodegradable drug delivery composition.

A surprising aspect of the biodegradable drug delivery compositions disclosed herein is that they typically maintain a low viscosity both at room temperature prior to injection and following subcutaneous or intramuscular injection while providing desirable pharmacokinetic (PK) characteristics in-vivo. These beneficial PK characteristics include minimal burst and sustained release of the beneficial agent over time.

Certain non-limiting aspects of the disclosure are provided below:
1. A composition comprising:

a vehicle comprising
a biodegradable polymer present in an amount of from about 5% to about 40% by weight of the vehicle and
a hydrophobic solvent present in an amount of from about 95% to about 60% by weight of the vehicle; and

an insoluble beneficial agent complex dispersed in the vehicle, the insoluble beneficial agent complex having a solubility of less than 1 mg/mL in the vehicle at 25° C.,

wherein the composition has a zero shear viscosity less than 1,200 centipoise at 25° C., and

wherein the composition is not an emulsion.
2. A composition comprising:

a vehicle comprising
a biodegradable polymer present in an amount of from about 5% to about 40% by weight of the vehicle and
a hydrophobic solvent present in an amount of from about 95% to about 60% by weight of the vehicle; and

an insoluble beneficial agent complex dispersed in the vehicle, the insoluble beneficial agent complex having a solubility of less than 1 mg/mL in the vehicle at 25° C.,

wherein when 0.8 mL of the composition is placed in a 1 mL syringe at 25° C. fitted with a 0.5 inch needle with a gauge of 21 and 10 lbs of force are applied, at least 0.5 mL of the composition is ejected from the syringe in less than 10 seconds, and

wherein the composition is not an emulsion.
3. A composition comprising:

a vehicle comprising
a biodegradable polymer present in an amount of from about 5% to about 40% by weight of the vehicle and
a single solvent consisting of hydrophobic solvent present in an amount of from about 95% to about 60% by weight of the vehicle; and

an insoluble component comprising beneficial agent dispersed in the vehicle, the insoluble component having a solubility of less than 1 mg/mL in the vehicle at 25° C.,

wherein the composition has a zero shear viscosity less than 1,200 centipoise at 25° C., and

wherein the composition is not an emulsion.
4. The composition of 3, wherein the insoluble component comprises insoluble beneficial agent complex.
5. An injectable depot composition comprising:

a single-phase vehicle comprising
a biodegradable polymer present in an amount of from about 5% to about 30% by weight of the vehicle, and
a hydrophobic solvent present in an amount of from about 95% to about 70% by weight of the vehicle; and

an insoluble beneficial agent complex dispersed in the vehicle, wherein at least 99% of the beneficial agent complex is insoluble in the vehicle at 25° C.,

wherein the injectable depot composition has a zero shear viscosity less than 1200 centipoise at 25° C., and

wherein the injectable depot composition is not an emulsion.
6. The composition of any one of 1, 2, 4, or 5, wherein when 10 mg of the insoluble beneficial agent complex is dispersed and left to stand in 1 mL of a test solution of phosphate buffered saline at pH 7.4 at 37° C. for 24 hours, the amount of beneficial agent dissolved in the test solution is less than 60% of the beneficial agent in the 10 mg of insoluble beneficial agent complex.
7. The composition of any one of 1 to 6, wherein the composition is not a gel.
8. The composition of any one of 1 to 6, wherein the composition has a G″/G′ ratio of greater than or equal to 10.
9. The composition of any one of 1 to 8, wherein the biodegradable polymer has a weight average molecular weight ranging from 1000 Daltons to 20,000 Daltons and comprises an ionizable end group comprising at least one member selected from carboxyl, sulfonate, phosphate, amino, secondary amino, tertiary amino, and quaternary ammonium.
10. The composition of any one of 1 to 9, wherein the biodegradable polymer is selected from the group consisting of poly-lactides, poly-glycolides, poly-caprolactones, poly-butyrolactones, poly-valerolactones, and copolymers and terpolymers thereof.
11. The composition of any of 1 to 10, wherein the biodegradable polymer comprises at least one of polylactic acid and poly(lactic acid-co-glycolic acid).
12. The composition of any one of 1 to 11, wherein the hydrophobic solvent comprises at least one member selected from benzyl alcohol, methyl benzoate, ethyl benzoate, n-propyl benzoate, isopropyl benzoate, butyl benzoate, isobutyl benzoate, sec-butyl benzoate, tert-butyl benzoate, isoamyl benzoate, and benzyl benzoate.
13. The composition of any one of 1 to 11, wherein the hydrophobic solvent comprises benzyl benzoate.
14. The composition of any one of 1 to 13, further comprising benzyl alcohol.
15. The composition of any one of 1 to 14, further comprising ethanol.
16. The composition of any one of 1, 2, and 4 to 15, wherein the insoluble beneficial agent complex comprises beneficial agent, a divalent metal ion, and one of a polymeric cationic complexing agent and a polymeric anionic complexing agent.
17. The composition of any one of 1, 2, and 4 to 16, wherein the insoluble beneficial agent complex comprises at least one member selected from the group consisting of protamine, poly-lysine, poly-arginine, polymyxin, carboxy-methyl-cellulose (CMC), poly-adenosine, and poly-thymine.
18. The composition of any one of 1, 2, and 4 to 17, wherein the insoluble beneficial agent complex is in the form of charge-neutralized particles.
19. The composition of any one of 1, 2, and 4 to 18, wherein the insoluble beneficial agent complex comprises beneficial agent and protamine.
20. The composition of any one of 1, 2, and 4 to 19, wherein the insoluble beneficial agent complex comprises beneficial agent and divalent metal or salt thereof.
21. The composition of 20, wherein the divalent metal is selected from Zn²⁺, Mg²⁺, and Ca²⁺.
22. The composition of any one of 1, 2, and 4 to 21, wherein the insoluble beneficial agent complex further comprises protamine.
23. The composition of any one of 1, 2, and 4 to 22, wherein the insoluble beneficial agent complex comprises beneficial agent and protamine, wherein the molar ratio of the beneficial agent and protamine is approximately 1:0.1 to 0.5.
24. The composition of any one of 1, 2, and 4 to 23, wherein the insoluble beneficial agent complex comprises beneficial agent, zinc, and protamine, wherein the molar ratio of the beneficial, zinc, and protamine is approximately 1:0.4 to 2:0.1 to 0.5.
25. The composition of any one of 1, 2, and 4 to 24, wherein the mean residence time (MRT) of beneficial agent in-vivo is greater than the sum of MRTₛₒₗᵥₑₙₜ+ΔMRT_{complex}+ΔMRT_{polymer}, wherein MRTₛₒₗᵥₑₙₜ is the MRT for the beneficial agent in the hydrophobic solvent alone, ΔMRT_{complex} is the change in MRT due to the insoluble beneficial agent complex in the absence of polymer, and ΔMRT_{polymer} is the change in MRT due to the polymer in the absence of complexation of the beneficial agent.
26. The composition of 25, wherein the MRT of the beneficial agent is up to 10 fold greater than the sum of MRTₛₒₗᵥₑₙₜ+ΔMRT_{complex}+ΔMRT_{polymer}.
27. The composition of any one of 1 to 26, wherein the composition forms a surface layer surrounding a liquid core following injection into phosphate buffered saline at pH 7.4 at 37° C., the surface layer having a thickness less than 10 μm.
28. The composition of any one of 1 to 27, wherein the vehicle consists of a single solvent consisting of the hydrophobic solvent consisting of benzyl benzoate, and the insoluble beneficial agent complex comprises beneficial agent and protamine.
29. The composition of 28, wherein the insoluble beneficial agent complex further comprises zinc.
30. A method of administering a beneficial agent to a subject, comprising administering to the subject via injection the composition of any one of 1 to 29.
31. A composition comprising:

a vehicle comprising
a biodegradable polymer present in an amount of from about 5% to about 40% by weight of the vehicle and
a hydrophobic solvent present in an amount of from about 95% to about 60% by weight of the vehicle; and

an insoluble beneficial agent complex dispersed in the vehicle, the insoluble beneficial agent complex having a solubility of less than 1 mg/mL in the vehicle at 25° C.,

wherein the composition has a zero shear viscosity less than 1,200 centipoise at 25° C., and

wherein the composition is not an emulsion.
32. The composition of 31, wherein the polymer is present in an amount of from about 10% to about 25% by weight of the vehicle.
33. The composition of 31, wherein the polymer is present in an amount of from about 15% to about 20% by weight of the vehicle.
34. The composition of any one of 31 to 33, wherein the hydrophobic solvent is present in an amount of from about 90% to about 75% by weight of the vehicle.
35. The composition of any one of 31 to 34, wherein the hydrophobic solvent is present in an amount of from about 85% to about 80% by weight of the vehicle.
36. The composition of any one of 31 to 35, wherein the hydrophobic solvent is a combination of two or more hydrophobic solvents.
37. The composition of any one of 31 to 36, wherein the composition has a zero shear viscosity less than 1,000 centipoise at 25° C.
38. The composition of any one of 31 to 37, wherein the composition has a zero shear viscosity less than 500 centipoise at 25° C.
39. The composition of any one of 31 to 38, wherein the composition has a zero shear viscosity less than 100 centipoise at 25° C.
40. The composition of any one of 31 to 39, wherein the vehicle maintains a zero shear viscosity which does not deviate by more than an order of magnitude for a period of at least one week when maintained at 37° C. for said period, wherein the zero shear viscosity is measured at a temperature of 37° C. following injection of 1 mL of the vehicle into 100 mL of phosphate buffered saline (PBS) at pH 7.4.
41. The composition of any one of 31 to 40, wherein when 0.8 mL of the composition is placed in a 1 mL syringe at 25° C. fitted with a 0.5 inch needle with a gauge of 21 and 10 lbs of force are applied, at least 0.5 mL of the composition is ejected from the syringe in less than 25 seconds.
42. The composition of 41, wherein the time period is less than 10 seconds.
43. The composition of 41, wherein the time period is less than 5 seconds.
44. The composition of any one of 31 to 43, wherein the composition is capable of being injected using a needless injector.
45. The composition of any one of 31 to 44, wherein the composition is not a gel.
46. The composition of any one of 31 to 45, wherein the composition does not form a gel when maintained at 37° C. for 7 days.
47. The composition of any one of 31 to 46, wherein the composition does not swell when contacted with water at 37° C. for 7 days.
48. The composition of any one of 31 to 47, wherein the biodegradable polymer comprises at least one member selected from poly-lactide, poly-glycolide, poly-caprolactone, and copolymers and terpolymers thereof.
49. The composition of any one of 31 to 48, wherein the biodegradable polymer is a terpolymer.
50. The composition of any one of 31 to 48, wherein the biodegradable polymer comprises polylactic acid (PLA).
51. The composition of 50, wherein the PLA comprises an ionizable end-group.
52. The composition of 51, wherein the ionizable end group is an acid end group.
53. The composition of 50, wherein the PLA comprises an unionizable end-group.
54. The composition of 53, wherein the unionizable end-group comprises at least one member selected from hydroxyl and ester.
55. The composition of any one of 31 to 48, wherein the biodegradable polymer comprises poly(lactic-co-glycolic acid) (PLGA).
56. The composition of 55, wherein the PLGA comprises an ionizable end-group.
57. The composition of 56, wherein the ionizable end-group is an acid end-group.
58. The composition of 55, wherein the PLGA comprises an unionizable end-group.
59. The composition of 58, wherein the unionizable end-group comprises at least one member selected from hydroxyl and ester.
60. The composition of 48, wherein the biodegradable polymer comprises a hydroxycaproic acid-glycolic acid-lactic acid terpolymer.
61. The composition of any one of 31 to 60, wherein the hydrophobic solvent has solubility in water of less than or equal to 5% by weight at 25° C.
62. The composition of 61, wherein the hydrophobic solvent has solubility in water of less than or equal to 1% by weight at 25° C.
63. The composition of any one of 31 to 60, wherein the solubility of water in the hydrophobic solvent is less than or equal to 10% by weight at 25° C.
64. The composition of any one of 31 to 60, wherein the solubility of water in the hydrophobic solvent is less than or equal to 5% by weight at 25° C.
65. The composition of any one of 31 to 60, wherein the solubility of water in the hydrophobic solvent is less than or equal to 1% by weight at 25° C.
66. The composition of any one of 31 to 60, wherein the hydrophobic solvent comprises a combination of two or more hydrophobic solvents.
67. The composition of any one of 31 to 60, wherein the hydrophobic solvent comprises one or more solvents selected from methyl benzoate, ethyl benzoate, n-propyl benzoate, isopropyl benzoate, butyl benzoate, isobutyl benzoate, sec-butyl benzoate, tert-butyl benzoate, isoamyl benzoate, benzyl benzoate and benzyl alcohol.
68. The composition of any one of 31 to 60, wherein the hydrophobic solvent is benzyl alcohol.
69. The composition of any one of 31 to 60, wherein the composition is free of benzyl alcohol.
70. The composition of any one of 31 to 60, wherein the hydrophobic solvent is benzyl benzoate.
71. The composition of any one of 31 to 70, wherein the composition comprises at least one additional solvent.
72. The composition of 71, wherein the at least one additional solvent is benzyl alcohol.
73. The composition of 71, wherein the at least one additional solvent is triacetin.
74. The composition of 71, wherein the at least one additional solvent is ethyl lactate.
75. The composition of 71, wherein the at least one additional solvent is ethanol.
76. The composition of any one of 31 to 65, wherein the composition does not comprise more than one solvent.
77. The composition of any one of 31 to 76, wherein the insoluble beneficial agent complex is charge-neutralized.
78. The composition of any one of 31 to 77, wherein the insoluble beneficial agent complex comprises protamine.
79. The composition of any one of 31 to 78, wherein the insoluble beneficial agent complex comprises a divalent metal salt of the beneficial agent.
80. The composition of 79, wherein the divalent metal comprises at least one member selected from Zn²⁺, Mg²⁺ and Ca²⁺.
81. The composition of any one of 31 to 80, wherein the insoluble beneficial agent complex comprises protamine and a Zn²⁺ salt of the beneficial agent.
82. The composition of any one of 31 to 78, wherein the insoluble beneficial agent complex comprises a beneficial agent and a cationic agent.
83. The composition of 82, wherein the cationic agent is selected from the group consisting of poly-lysine, poly-arginine, and polymyxin.
84. The composition of any one of 31 to 78, wherein the insoluble beneficial agent complex comprises a beneficial agent and an anionic agent.
85. The composition of 84, wherein the anionic agent comprises at least one member selected from carboxy-methyl-cellulose (CMC), a poly-adenosine, and a poly-thymine.
86. The composition of 84, wherein the anionic agent is at least a 10mer poly-adenosine or poly-thymine.
87. The composition of 86, wherein the anionic agent is at least a 20mer poly-adenosine or poly-thymine.
88. The composition of 87, wherein the anionic agent is at least a 150mer poly-adenosine or poly-thymine.
89. The composition of 88, wherein the anionic agent is at least a 1500mer poly-thymine.
90. The composition of any one of 31 to 89, wherein the composition further comprises methionine.
91. The composition of 31 to 90, wherein the insoluble beneficial agent complex is dispersed in the vehicle in the form of particles having an average size ranging from about 1 μm to about 400 μm.
92. The composition of 91, wherein the insoluble beneficial agent complex is dispersed in the vehicle in the form of particles having an average size ranging from about 1 μm to about 10 μm.
93. The composition of 91, wherein the insoluble beneficial agent complex is dispersed in the vehicle in the form of particles having an average size ranging from about 10 μm to about 100 μm.
94. The composition of 91, wherein the apparent density of the vehicle is within 10% of the apparent density of the particles.
95. The composition of any one of 31 to 94, wherein when 10 mg of the insoluble beneficial agent complex is dispersed and left to stand in 1 mL of a test solution of phosphate buffered saline at pH 7.4 at 37° C. for 24 hours, the amount of beneficial agent dissolved in the test solution is not more than 50% of the beneficial agent in the 10 mg of insoluble beneficial agent complex.
96. The composition of any one of 31 to 95, wherein the insoluble beneficial agent complex comprises beneficial agent and protamine, wherein the molar ratio of the beneficial agent and protamine is approximately 1:0.1 to 0.5.
97. The composition of any one of 31 to 81, wherein the insoluble beneficial agent complex comprises beneficial agent, zinc, and protamine, wherein the molar ratio of the beneficial agent, zinc, and protamine is approximately 1:0.4 to 2:0.1 to 0.5.
98. The composition of 90, wherein the insoluble beneficial agent complex comprises a peptide or a protein as the beneficial agent and the composition maintains a purity of about 90% or greater for a period of at least 24 hours following exposure to gamma irradiation at a dose of 25 kGy.
99. The composition of 98, wherein the period is at least one month.
100. The composition of 98, wherein the insoluble beneficial agent complex comprises a peptide or a protein as the beneficial agent and the composition maintains a purity of about 95% or greater for a period of at least 24 hours following exposure to gamma irradiation at a dose of 25 kGy.
101. The composition of 100, wherein the period is at least one month.
102. The composition of any one of 31 to 101, wherein the vehicle further comprises sucrose acetate isobutyrate (SAIB) in an amount of from about 5% to about 20% by weight of the vehicle.
103. The composition of 102, wherein the vehicle comprises SAIB in an amount of from about 5% to about 10% by weight of the vehicle.
104. The composition of 103, wherein the vehicle comprises about 5% to 10% SAIB, about 70% to about 75% of the hydrophobic solvent, and about 15% to 25% of the biodegradable polymer, wherein each % is % by weight of the vehicle.
105. The composition of 104, wherein the insoluble beneficial agent complex comprises a Zn²⁺ salt of the beneficial agent.
106. The composition of 103, wherein the vehicle comprises about 5 to about 10% SAIB, about 65% to about 70% benzyl benzoate, about 3% to about 7% ethanol, and about 15% to about 25% poly(lactic-co-glycolic acid) (PLGA), wherein each % is % by weight of the vehicle.
107. The composition of 102, wherein the vehicle comprises about 15% to about 25% SAIB, about 55% to about 65% benzyl benzoate, about 5% to about 15% benzyl alcohol, and about 5% to about 15% polylactic acid (PLA), wherein each % is % by weight of the vehicle.
108. The composition of 107, wherein the insoluble beneficial agent complex comprises a Zn²⁺ salt of the beneficial agent.
109. The composition of 102, wherein the vehicle comprises about 65% to about 75% benzyl benzoate, about 5% to about 15% benzyl alcohol, and about 15% to about 25% polylactic acid (PLA), wherein each % is % by weight of the vehicle.
110. The composition of 102, wherein the insoluble beneficial agent complex comprises a Zn²⁺ salt of the beneficial agent.
111. The composition of 110, wherein the amount of the insoluble beneficial agent complex ranges from about 1% to about 50% by weight of the composition.
112. The composition of any one of 31 to 111, wherein the insoluble beneficial agent complex comprises a beneficial agent comprising at least one member selected from a protein, a peptide, a nucleic acid, a nucleotide, a nucleoside, and precursors, derivatives, prodrugs and analogues thereof.
113. The composition of 112, wherein the insoluble beneficial agent complex comprises a beneficial agent comprising a protein.
114. The composition of 113, wherein the protein is IFNα2a or recombinant human rhIFNα2a.
115. The composition of 113, wherein the protein is a growth hormone.
116. The composition of 115, wherein the growth hormone is human growth hormone (hGH) or recombinant human growth hormone (rhGH).
117. The composition of 112, wherein the insoluble beneficial agent complex comprises a beneficial agent comprising a peptide.
118. The composition of 117, wherein the peptide is Glucagon-like peptide-1 (GLP-1) or an analogue thereof.
119. The composition of 117, wherein the peptide is exenatide.
120. The composition of 31 to 111, wherein the insoluble beneficial agent complex comprises a beneficial agent comprising an antibody or a fragment thereof.
121. The composition of 31 to 111, wherein the insoluble beneficial agent complex comprises a beneficial agent comprising a nucleotide, nucleoside, or an analogue thereof.
122. The composition of 121, wherein the insoluble beneficial agent complex comprises a beneficial agent comprising a nucleoside analogue.
123. The composition of 122, wherein the nucleoside analogue is azacytidine.
124. The composition of 31 to 111, wherein the insoluble beneficial agent complex comprises a beneficial agent comprising a low molecular weight compound.
125. The composition of 124, wherein the low molecular weight compound comprises an antineoplastic agent.
126. The composition of 125, wherein the antineoplastic agent is bortezomib.
127. The composition of any one of 31 to 126, wherein the composition forms a surface layer surrounding a liquid core following injection into phosphate buffered saline at pH 7.4 at 37° C., the surface layer having a thickness less than 10 μm.
128. An injectable depot composition comprising:

a single-phase vehicle comprising
a biodegradable polymer present in an amount of from about 5% to about 30% by weight of the vehicle, and
a hydrophobic solvent present in an amount of from about 95% to about 70% by weight of the vehicle; and

an insoluble beneficial agent complex dispersed in the vehicle, wherein at least 99% of the beneficial agent complex is insoluble in the vehicle at 25° C., wherein the injectable depot composition has a zero shear viscosity less than 1200 centipoise at 25° C., and

wherein the injectable depot composition is not an emulsion.
129. The composition of 128, wherein the biodegradable polymer comprises polylactic acid.
130. A composition comprising:

a vehicle comprising
a biodegradable polymer present in an amount of from about 5% to about 40% by weight of the vehicle and
a hydrophobic solvent present in an amount of from about 95% to about 60% by weight of the vehicle; and

an insoluble beneficial agent complex dispersed in the vehicle, the insoluble beneficial agent complex having a solubility of less than 1 mg/mL in the vehicle at 25° C.,
wherein the mean residence time (MRT) of the beneficial agent in-vivo is greater than the sum of MRTₛₒₗᵥₑₙₜ+ΔMRT_{complex}+ΔMRT_{polymer}, wherein MRTₛₒₗᵥₑₙₜ is the MRT for the beneficial agent in the hydrophobic solvent alone, ΔMRT_{complex} is the change in MRT due to the insoluble beneficial agent complex, in the absence of polymer, and ΔMRT_{polymer} is the change in MRT due to the polymer, in the absence of complexation of the beneficial agent.
131. The composition of 130, wherein the MRT of the beneficial agent is up to 10 fold greater than the sum of MRTₛₒₗᵥₑₙₜ+ΔMRT_{complex}+ΔMRT_{polymer}.
132. A composition comprising:

a vehicle comprising
a biodegradable polymer present in an amount of from about 5% to about 40% by weight of the vehicle and
a hydrophobic solvent present in an amount of from about 95% to about 60% by weight of the vehicle; and

an insoluble component comprising beneficial agent dispersed in the vehicle, the insoluble component having a solubility of less than 1 mg/mL in the vehicle at 25° C., wherein the biodegradable polymer comprises an ionizable end-group.
133. The composition of any one of 128 to 132, wherein the composition forms a surface layer surrounding a liquid core following injection into phosphate buffered saline at pH 7.4 at 37° C., the surface layer having a thickness less than 10 μm.
134. A composition comprising:

a vehicle comprising
a biodegradable polymer present in an amount of from about 5% to about 40% by weight of the vehicle and
a hydrophobic solvent present in an amount of from about 95% to about 60% by weight of the vehicle; and

an insoluble component comprising beneficial agent dispersed in the vehicle, the insoluble component having a solubility of less than 1 mg/mL in the vehicle at 25° C., wherein the biodegradable polymer has a weight average molecular weight ranging from 1000 Daltons to 11,000 Daltons.
135. A composition comprising:

a vehicle comprising
a biodegradable polymer present in an amount of from about 5% to about 40% by weight of the vehicle and
a hydrophobic solvent present in an amount of from about 95% to about 60% by weight of the vehicle; and

an insoluble beneficial agent complex dispersed in the vehicle, the insoluble beneficial agent complex having a solubility of less than 1 mg/mL in the vehicle at 25° C.,

wherein when 0.8 mL of the composition is placed in a 1 mL syringe at 25° C. fitted with a 0.5 inch needle with a gauge of 21 and 10 lbs of force are applied, at least 0.5 mL of the composition is ejected from the syringe in less than 10 seconds, and

wherein the composition is not an emulsion.
136. A composition comprising:

a vehicle comprising
a biodegradable polymer present in an amount of from about 5% to about 40% by weight of the vehicle, wherein the biodegradable polymer comprises an ionizable end group, and
a hydrophobic solvent present in an amount of from about 95% to about 60% by weight of the vehicle; and

an insoluble component comprising beneficial agent dispersed in the vehicle, the insoluble component having a solubility of less than 1 mg/mL in the vehicle at 25° C.,

wherein the composition has a zero shear viscosity less than 500 centipoise at 25° C., and

wherein the composition is not a gel.
137. The composition of 136, wherein the composition has a G″/G′ ratio of greater than or equal to 10.
138. A composition comprising:

a vehicle comprising
a biodegradable polymer present in an amount of from about 5% to about 40% by weight of the vehicle and
a single solvent consisting of hydrophobic solvent present in an amount of from about 95% to about 60% by weight of the vehicle; and

an insoluble component comprising beneficial agent dispersed in the vehicle, the insoluble component having a solubility of less than 1 mg/mL in the vehicle at 25° C.,

wherein the composition has a zero shear viscosity less than 1,200 centipoise at 25° C., and

wherein the composition is not an emulsion.
139. The composition of 138, wherein the composition has a G″/G′ ratio of greater than or equal to 10.
140. A composition comprising:

a vehicle comprising
a biodegradable polymer present in an amount of from about 5% to about 40% by weight of the vehicle, and
a single solvent consisting of a hydrophobic solvent present in an amount of from about 95% to about 60% by weight of the vehicle; and

an insoluble beneficial agent complex dispersed in the vehicle, the insoluble beneficial agent complex having a solubility of less than 1 mg/mL in the vehicle at 25° C., the insoluble beneficial agent comprising a beneficial agent, a metal, and one of a cationic agent and an anionic agent,

wherein the composition has a zero shear viscosity less than 500 centipoise at 25° C., and

wherein the composition is not a gel.
141. The composition of 140, wherein the composition has a G″/G′ ratio of greater than or equal to 10.
142. A composition comprising:

a vehicle comprising
a biodegradable polymer present in an amount of from about 5% to about 40% by weight of the vehicle, the biodegradable polymer being polylactic acid or poly(lactic acid-co-glycolic acid), and
a hydrophobic benzoate solvent present in an amount of from about 95% to about 60% by weight of the vehicle; and

an insoluble beneficial agent complex dispersed in the vehicle, the insoluble component having a solubility of less than 1 mg/mL in the vehicle at 25° C., the insoluble beneficial agent complex comprising a beneficial agent, zinc, and protamine,

wherein the composition is not a gel.
143. The composition of 142, wherein the composition has a G″/G′ ratio of greater than or equal to 10.
144. A polymer comprising at least one monomer selected from lactic acid, glycolic acid, hydroxybutyric acid, hydroxyvaleric acid, and hydroxycaproic acid, wherein the polymer has a weight average molecular weight ranging from 1000 Daltons to 11,000 Daltons, and wherein the polymer comprises ionizable end groups.
145. The polymer of 144, wherein the weight average molecular weight ranges from 1500 Daltons to 10,500 Daltons.
146. The polymer of 144, wherein the weight average molecular weight ranges from 2000 Daltons to 10,000 Daltons.
147. The polymer of 144, wherein the weight average molecular weight ranges from 2500 Daltons to 9500 Daltons.
148. The polymer of 144, wherein the ionizable end groups comprise at least one member selected from carboxyl, sulfonate, phosphate, amino, secondary amino, tertiary amino, and quaternary ammonium.
149. The polymer of 144, wherein the ionizable end groups comprise carboxyl.
150. A composition comprising:

a vehicle comprising
a biodegradable polymer present in an amount of from about 5% to about 40% by weight of the vehicle and
a hydrophobic solvent present in an amount of from about 95% to about 60% by weight of the vehicle; and

an insoluble component comprising beneficial agent dispersed in the vehicle, the insoluble component having a solubility less than 1 mg/mL in the vehicle at 25° C.,

wherein the composition has a zero shear viscosity less than 1,200 centipoise at 25° C.,

wherein the composition forms a surface layer surrounding a liquid core following injection into phosphate buffered saline at pH 7.4 at 37° C., the surface layer having a thickness less than 10 μm, and

wherein the composition is not an emulsion.
151. A composition comprising:

a vehicle comprising
a biodegradable polymer present in an amount of from about 5% to about 40% by weight of the vehicle, the biodegradable polymer being polylactic acid or poly(lactic acid-co-glycolic acid), and
a hydrophobic benzoate solvent present in an amount of from about 95% to about 60% by weight of the vehicle; and

an insoluble component comprising beneficial agent dispersed in the vehicle, the insoluble component having a solubility of less than 1 mg/mL in the vehicle at 25° C., the insoluble beneficial agent comprising a beneficial agent, zinc, and protamine,

wherein the composition forms a surface layer surrounding a liquid core following injection into phosphate buffered saline at pH 7.4 at 37° C., the surface layer having a thickness less than 10 μm.
152. A method of making a composition, comprising:

combining a biodegradable polymer and a hydrophobic solvent to form a vehicle, wherein the biodegradable polymer is included in an amount of from about 5% to about 40% by weight of the vehicle, and the hydrophobic solvent is included in an amount of from about 95% to about 60% by weight of the vehicle; and

dispersing an insoluble beneficial agent complex in the vehicle, wherein the insoluble beneficial agent complex has a solubility of less than 1 mg/mL in the vehicle at 25° C., thereby providing a composition having a zero shear viscosity less than 1,200 centipoise at 25° C., which composition is not an emulsion.
153. The method of 152, wherein the polymer is included in an amount of from about 10% to about 25% by weight of the vehicle.
154. The method of 153, wherein the polymer is included in an amount of from about 15% to about 20% by weight of the vehicle.
155. The method of any one of 152 to 154, wherein the hydrophobic solvent is included in an amount of from about 90% to about 75% by weight of the vehicle.
156. The method of 155, wherein the hydrophobic solvent is included in an amount of from about 85% to about 80% by weight of the vehicle.
157. The method of any one of 152 to 156, wherein the hydrophobic solvent is a combination of two or more hydrophobic solvents.
158. The method of any one of 152 to 157, wherein the composition has a zero shear viscosity less than 1,000 centipoise at 25° C.
159. The method of 158, wherein the composition has a zero shear viscosity less than 500 centipoise at 25° C.
160. The method of 159, wherein the composition has a zero shear viscosity less than 100 centipoise at 25° C.
161. The method of any one of 152 to 160, wherein the vehicle maintains a zero shear viscosity which does not deviate by more than an order of magnitude for a period of one week when maintained at 37° C. for said period and when measured at any time point during the one week period, wherein the zero shear viscosity is measured at a temperature of 37° C. following injection of about 1 mL of the vehicle into 100 mL of phosphate buffered saline (PBS) at pH 7.4.
162. The method of any one of 152 to 160, wherein when 0.8 mL of the composition is placed in a 1 mL syringe at 25° C. fitted with a 0.5 inch needle with a gauge of 21 and 10 lbs of force are applied, at least 0.5 mL of the composition is ejected from the syringe in less than 25 seconds.
163. The method of 162, wherein the time period is less than 10 seconds.
164. The method of 163, wherein the time period is less than 5 seconds.
165. The method of any one of 152 to 164, wherein the composition is capable of being injected using a needless injector.
166. The method of any one of 152 to 165, wherein the biodegradable polymer comprises at least one member selected from poly-lactides, poly-glycolides, poly-caprolactones and copolymers and terpolymers thereof.
167. The method of any one of 152 to 166, wherein the biodegradable polymer is a terpolymer.
168. The method of any one of 152 to 166, wherein the biodegradable polymer comprises polylactic acid (PLA).
169. The method of 168, wherein the PLA comprises an ionizable end-group.
170. The method of 169, wherein the ionizable end group is an acid end group.
171. The method of 168, wherein the PLA comprises an unionizable end-group.
172. The method of 171, wherein the unionizable end-group comprises at least one member selected from hydroxyl and ester.
173. The method of any one of 152 to 166, wherein the biodegradable polymer comprises poly(lactic-co-glycolic acid) (PLGA).
174. The method of 173, wherein the PLGA comprises an ionizable end-group.
175. The method of 174, wherein the ionizable end-group is an acid end-group.
176. The method of 173, wherein the PLGA comprises an unionizable end-group.
177. The method of 176, wherein the unionizable end-group comprises at least one member selected from hydroxyl and ester.
178. The method of 152, wherein the biodegradable polymer comprises a hydroxycaproic acid-glycolic acid-lactic acid terpolymer.
179. The method of any one of 152 to 156, wherein the hydrophobic solvent has solubility in water of less than or equal to 5% by weight at 25° C.
180. The method of 179, wherein the hydrophobic solvent has solubility in water of less than or equal to 1% by weight at 25° C.
181. The method of any one of 152 to 156, wherein the solubility of water in the hydrophobic solvent is less than or equal to 10% by weight at 25° C.
182. The method of 181, wherein the solubility of water in the hydrophobic solvent is less than or equal to 5% by weight at 25° C.
183. The method of 182, wherein the solubility of water in the hydrophobic solvent is less than or equal to 1% by weight at 25° C.
184. The method of any one of 152 to 183, wherein the composition is free of hydrophilic solvent.
185. The method of any one of 152 to 184, wherein the hydrophobic solvent comprises at least one member selected from methyl benzoate, ethyl benzoate, n-propyl benzoate, isopropyl benzoate, butyl benzoate, isobutyl benzoate, sec-butyl benzoate, tert-butyl benzoate, isoamyl benzoate, benzyl benzoate and benzyl alcohol.
186. The method of any one of 152 to 185, wherein the hydrophobic solvent is benzyl alcohol.
187. The method of any one of 152 to 185, wherein the hydrophobic solvent is triethyl citrate.
188. The method of any one of 152 to 185, wherein the hydrophobic solvent is benzyl benzoate.
189. The method of any one of 152 to 188, wherein the composition comprises at least one additional solvent.
190. The method of 189, wherein the at least one additional solvent is benzyl alcohol.
191. The method of 189, wherein the at least one additional solvent is triacetin.
192. The method of 189, wherein the at least one additional solvent is ethyl lactate.
193. The method of 189, wherein the at least one additional solvent is ethanol.
194. The method of any one of 152 to 193 wherein the insoluble beneficial agent complex is charge-neutralized.
195. The method of any one of 152 to 194, wherein the insoluble beneficial agent complex comprises protamine.
196. The method of any one of 152 to 195, wherein the insoluble beneficial agent complex comprises a divalent metal salt of the beneficial agent.
197. The method of 196, wherein the divalent metal comprises at least one member selected from Zn²⁺, Mg²⁺, and Ca²⁺.
198. The method of any one of 152 to 197, wherein the insoluble beneficial agent complex comprises protamine and a Zn²⁺ salt of the beneficial agent.
199. The method of any one of 152 to 195, wherein the insoluble beneficial agent complex comprises a beneficial agent and a cationic agent.
200. The method of 199, wherein the cationic agent comprises at least one member selected from poly-lysine, poly-arginine, and polymyxin.
201. The method of any one of 152 to 195, wherein the insoluble beneficial agent complex comprises a beneficial agent and an anionic agent.
202. The method of 201, wherein the anionic agent comprises at least one member selected from carboxy-methyl-cellulose (CMC), a poly-adenosine, and a poly-thymine.
203. The method of 201, wherein the anionic agent is at least a 10mer poly-adenosine or poly-thymine.
204. The method of 203, wherein the anionic agent is at least a 20mer poly-adenosine or poly-thymine.
205. The method of 204, wherein the anionic agent is at least a 150mer poly-adenosine or poly-thymine.
206. The method of 205, wherein the anionic agent is at least a 1500mer poly-thymine.
207. The method of any one of 152 to 206, wherein the composition comprises methionine.
208. The method of any one of 152 to 207, comprising dispersing the insoluble beneficial agent complex in the vehicle in the form of particles having sizes ranging from about 1 μm to about 400 μm.
209. The method of 208, wherein the insoluble beneficial agent complex is dispersed in the vehicle in the form of particles having sizes ranging from about 1 μm to about 10 μm.
210. The method of 208, wherein the insoluble beneficial agent complex is dispersed in the vehicle in the form of particles having sizes ranging from about 10 μm to about 100 μm.
211. The method of 209, comprising forming the particles by spray-drying.
212. The method of 208, 209 or 210, comprising forming the particles by freeze-drying.
213. The method of 208, wherein the apparent density of the vehicle is within 10% of the apparent density of the particles.
214. The method of any one of 152 to 213, wherein the vehicle further comprises sucrose acetate isobutyrate (SAIB) in an amount of from about 5% to about 20% by weight of the vehicle.
215. The method of 214, wherein the vehicle comprises SAIB in an amount of from about 6% to about 10% by weight of the vehicle.
216. The method of 215, wherein the vehicle comprises about 5% to about 10% SAIB, about 70% to about 75% of the hydrophobic solvent, and about 15% to about 25% of the biodegradable polymer, wherein each % is % by weight of the vehicle.
217. The method of 216, wherein the beneficial agent complex comprises a Zn²⁺ salt of the beneficial agent.
218. The method of 215, wherein the vehicle comprises about 5% to about 10% SAIB, about 65% to about 70% benzyl benzoate, about 3% to about 7% ethanol, and about 15% to about 25% poly(lactic-co-glycolic acid) (PLGA), wherein each % is % by weight of the vehicle.
219. The method of 218, wherein, wherein the beneficial agent complex comprises a Zn²⁺ salt of the beneficial agent.
220. The method of 214, wherein the vehicle comprises about 15% to about 25% SAIB, about 55% to about 65% benzyl benzoate, about 5% to about 15% benzyl alcohol, and about 5% to about 15% polylactic acid (PLA), wherein each % is % by weight of the vehicle.
221. The method of 220, wherein the beneficial agent complex comprises a Zn²⁺ salt of the beneficial agent.
222. The method of 152, wherein the vehicle comprises about 65% to about 75% benzyl benzoate, about 5% to about 15% benzyl alcohol, and about 15% to about 25% polylactic acid (PLA), wherein each % is % by weight of the vehicle.
223. The method of 222, wherein the beneficial agent complex comprises protamine.
224. The method of 223, wherein the beneficial agent complex comprises a Zn²⁺ salt of the beneficial agent.
225. The method of 152, wherein an amount of the insoluble beneficial agent complex ranges from about 1% to about 50% by weight of the composition.
226. The method of any one of 152 to 225, wherein the insoluble beneficial agent complex comprises beneficial agent and protamine, wherein the molar ratio of the beneficial agent and protamine is approximately 1:0.1 to 0.5.
227. The method of any one of 152 to 198, wherein the insoluble beneficial agent complex comprises beneficial agent, zinc, and protamine, wherein the molar ratio of the beneficial agent, zinc, and protamine is approximately 1:0.4 to 2:0.1 to 0.5.
228. The method of any one of 152 to 227, wherein the insoluble beneficial agent complex comprises at least one beneficial agent selected from a protein, a peptide, a nucleic acid, a nucleotide, a nucleoside, and precursors, derivatives, prodrugs and analogues thereof.
229. The method of any one of 152 to 228, wherein the insoluble beneficial agent complex comprises a beneficial agent comprising a protein.
230. The method of 229, wherein the protein is IFNα2a or recombinant human rhIFNα2a.
231. The method of 229, wherein the protein is a growth hormone.
232. The method of 231, wherein the growth hormone is human growth hormone (hGH) or recombinant human growth hormone (rhGH).
233. The method of any one of 152 to 227, wherein the insoluble beneficial agent complex comprises a beneficial agent comprising an antibody or fragment thereof.
234. The method of 233, wherein the antibody is a monoclonal antibody or fragment thereof.
235. The method of 234, wherein the monoclonal antibody is adalimumab.
236. The method of 234, wherein the monoclonal antibody is bevacizumab.
237. The method of 234, wherein the monoclonal antibody is infliximab.
238. The method of 152 to 228, wherein the insoluble beneficial agent complex comprises a beneficial agent comprising a peptide.
239. The method of 238, wherein the peptide is Glucagon-like peptide-1 (GLP-1) or an analogue thereof.
240. The method of 238, wherein the peptide is exenatide.
241. The method of 152 to 228, wherein the insoluble beneficial agent complex comprises a beneficial agent comprising a nucleotide, nucleoside, or an analogue thereof.
242. The method of 241, wherein the insoluble beneficial agent complex comprises a beneficial agent comprising a nucleoside analogue.
243. The method of 242, wherein the nucleoside analogue is azacytidine.
244. The method of any one of 152 to 227, wherein the insoluble beneficial agent complex comprises a beneficial agent comprising a low molecular weight compound.
245. The method of 244, wherein the low molecular weight compound comprises an antineoplastic agent.
246. The method of 245, wherein the antineoplastic agent is bortezomib.
247. A method of administering a beneficial agent to a subject, comprising:

administering to the subject via injection a composition comprising
a single-phase vehicle comprising a biodegradable polymer present in an amount of from about 5% to about 40% by weight of the vehicle, and a hydrophobic solvent present in an amount of from about 95% to about 60% by weight of the vehicle; and
an insoluble beneficial agent complex dispersed in the vehicle, wherein the composition has a zero shear viscosity less than 1,200 centipoise at 25° C. and is not an emulsion.
248. The method of 247, wherein, following administration of the composition, the beneficial agent is present at detectable levels in the plasma of the subject for an extended period of time relative to administration of the drug alone or administration of the drug in the hydrophobic solvent alone.
249. The method of 247 or 248, wherein the composition is administered to the subject using a needle of 21 gauge or smaller.
250. The method of any one of 247 to 249, wherein the composition is administered to the subject using a 21 to 27 gauge needle.
251. The method of 247, wherein the composition is administered to the subject using a needless injector.
252. The method of any one of 247 to 251, wherein, following administration of the composition, the mean residence time (MRT) of the beneficial agent in-vivo is greater than the sum of sum of MRTₛₒₗᵥₑₙₜ+ΔMRT_{complex}+ΔMRT_{polymer}, wherein MRTₛₒₗᵥₑₙₜ is the MRT for the beneficial agent in the hydrophobic solvent alone, ΔMRT_{complex} is the change in MRT due to the insoluble beneficial agent complex in the absence of polymer, and ΔMRT_{polymer} is the change in MRT due to the polymer in the absence of complexation of the beneficial agent.
253. The method of 252, wherein the MRT of the beneficial agent is up to 10 fold greater than the sum of MRTₛₒₗᵥₑₙₜ+ΔMRT_{complex}+ΔMRT_{polymer}.
254. An injectable composition comprising:
   a vehicle comprising
      a biodegradable polymer present in an amount of from 5% to 30% by weight of the vehicle and
      a liquid hydrophobic solvent present in an amount of from 95% to 60% by weight of the vehicle; and
   a solid complex which comprises a beneficial agent, which complex is insoluble in the vehicle and dispersed in the vehicle.
255. An injectable composition according to 254, wherein the beneficial agent complex comprises a polymeric cationic complexing agent or a polymeric anionic complexing agent.
256. An injectable composition according to 255, wherein:
   the polymeric cationic complexing agent is selected from protamine, polylysine, polyarginine and polymyxin; or
   the polymeric anionic complexing agent is selected from carboxymethylcellulose, polyadenosine and polythymine.
257. An injectable composition according to any one of 254 to 256, wherein the biodegradable polymer is selected from poly-lactides, poly-glycolides, poly-caprolactones and copolymers and terpolymers thereof.
258. An injectable composition according to any one of 254 to 257, wherein the hydrophobic solvent is selected from benzyl alcohol, methyl benzoate, ethyl benzoate, n-propyl benzoate, isopropyl benzoate, butyl benzoate, isobutyl benzoate, sec-butyl benzoate, tert-butyl benzoate, isoamyl benzoate, benzyl benzoate and mixtures thereof.
259. An injectable composition according to any one of 254 to 258, wherein the composition satisfies at least one of the following (A) and (B):
   (A) the composition has a zero shear viscosity less than 1,200 centipoise at 25° C.; and
   (B) when 0.8 mL of the composition is placed in a 1 mL syringe at 25° C. fitted with a 0.5 inch needle with a gauge of 21 and 10 lbs of force are applied, at least 0.5 mL of the composition is ejected from the syringe in less than 25 seconds.
260. An injectable composition according to any one of 254 to 259, wherein the composition satisfies at least one of the following (C) and (D):
   (C) said insoluble beneficial agent complex has a solubility of less than 1 mg/mL in the vehicle at 25° C.; and
   (D) when 10 mg of the insoluble beneficial agent complex is dispersed and left to stand in 1 mL of a test solution of phosphate buffered saline at pH 7.4 at 37° C. for 24 hours, the amount of beneficial agent dissolved in the test solution is not more than 50% of the beneficial agent in the 10 mg of insoluble beneficial agent complex.
261. An injectable composition according to any one of 254 to 260, comprising:
   a vehicle comprising
      a biodegradable polymer present in an amount of from 5% to 40% by weight of the vehicle and which is selected from poly-lactides and poly(lactic acid-co-glycolic acid)s, and
      a liquid hydrophobic solvent present in an amount of from 95% to 60% by weight of the vehicle and which comprises benzyl benzoate; and
   a solid complex which comprises a beneficial agent, which complex is insoluble in the vehicle and dispersed in the vehicle and which complex comprises protamine.
262. An injectable composition according to any one of 254 to 261, wherein the beneficial agent complex comprises a divalent metal or salt thereof.
263. An injectable composition according to 262, wherein the divalent metal is selected from Zn²⁺, Mg²⁺, and Ca²⁺.
264. An injectable composition according to any one of 254 to 263, wherein the beneficial agent complex is in the form of charge-neutral particles.
265. An injectable composition according to any one of 254 to 264, wherein the biodegradable polymer comprises an ionizable end group.
266. An injectable composition according to any one of 254 to 265, wherein the composition is not an emulsion or a gel.
267. An injectable composition according to any one of 254 to 266, wherein the beneficial agent is a peptide.
268. An injectable composition according to any one of 254 to 266, wherein the beneficial agent is a growth hormone.
269. An injectable composition as defined in any one of 254 to 268 for use in a method of treatment of the human or animal body by therapy.
270. A method of making an injectable composition, comprising:
   combining a biodegradable polymer and a liquid hydrophobic solvent to form a vehicle, which vehicle comprises the biodegradable polymer in an amount of from 5% to 40% by weight of the vehicle and the liquid hydrophobic solvent in an amount of from 95% to 60% by weight of the vehicle; and dispersing in the vehicle a solid complex, which complex comprises a beneficial agent and which complex is insoluble in the vehicle.
271. An injectable composition obtainable by the method defined in 270.
272. A method of making a complex comprising:

contacting at least one of a protein and peptide with a cationic complexing agent at a pH greater than 8 to form a complex.
273. The method of 272, wherein the cationic complexing agent comprises at least one member selected from protamine, poly-lysine, poly-arginine, and polymyxin.
274. A method of making a complex comprising:

contacting at least one of a protein and peptide with an anionic complexing agent at a pH less than 3 to form a complex.
275. The method of 274, wherein the anionic complexing agent comprises at least one member selected from carboxy-methyl-cellulose, poly-adenosine, and poly-thymine.

### BRIEF DESCRIPTION OF THE DRAWINGS

The present invention is further described in the description of invention that follows, in reference to the noted plurality of non-limiting drawings, wherein:

FIG. 1 is a graph showing dose-normalized group average rhGH serum profiles for in-vivo experiments (Sprague-Dawley rats) conducted utilizing injectable depot compositions, including injectable, biodegradable drug delivery depots as disclosed herein.

FIG. 2 shows graphs of serum rhGH concentrations plotted over time for each of six animals for each of six drug delivery depot test groups: non-complexed rhGH in aqueous solution (top left), rhGH-protamine complex suspended in aqueous medium (top middle), rhGH-protamine complex in Benzyl Benzoate (BB) (top right), rhGH-protamine complex in sucrose acetate isobutryate (SAIB):BB vehicle (bottom left), rhGH-protamine complex in BB:poly lactic acid (PLA) vehicle (bottom middle), and rhGH-protamine complex in SAIB:BB:PLA vehicle (bottom right).

FIG. 3 is a graph showing IFN-α2a serum concentration in individual rats over a 96 hr period following subcutaneous injection of a 2.5 mg/ml FNα2a formulation with 1% sucrose (w/w) and protamine-zinc (spray dried), in a SAIB/BB/PLA (8:72:20, % w/w) vehicle. The IFN-α2a beneficial agent is provided as a beneficial agent complex with zinc and protamine.

FIG. 4 is a graph showing IFN-α2a serum concentration in individual rats over a 96 hr period following subcutaneous injection of a 2.5 mg/ml FNα2a formulation with 1% sucrose (w/w) and protamine-zinc (spray dried), in a SAIB/BB/PLGA (8:72:20, % w/w) vehicle The IFN-α2a beneficial agent is provided as a beneficial agent complex with zinc and protamine.

FIG. 5 is a graph showing average serum concentration over time for the formulations referenced in FIGS. 3 and 4.

FIG. 6 is a graph showing IFNα2a serum concentration in individual rats over time following a 500 SC bolus of a 20 mg/ml IFNα2a-protamine (1:0.3 m/m) formulation with 1% sucrose, in a SAIB/BB/PLA (8:72:20, % w/w) vehicle. Serum concentrations were determined via Enzyme-Linked Immunosorbent Assay (ELISA).

FIG. 7 is a graph showing IFNα2a serum concentration in individual rats over time following a 500 SC bolus of a 20 mg/ml FNα2a, 1% CMC, 1% sucrose in a SAIB/BB/PLA (8:72:20, % w/w) vehicle. Serum concentrations were determined via ELISA. The IFN-α2a beneficial agent is provided as a beneficial agent complex with carboxy methyl cellulose (CMC).

FIG. 8 is a graph showing IFNα2a serum concentration in individual primates over time following dosing at 2 mg/kg using a 40 mg/ml FNα2a-protamine formulation with sucrose, in a SAIB/BB/PLA (8:72:20, % w/w) vehicle.

FIG. 9 is a graph showing IFNα2a serum concentration in individual primates over time following dosing at 2 mg/kg using a 40 mg/ml FNα2a-CMC formulation with sucrose, in a SAIB/BB/PLA (8:72:20, % w/w) vehicle.

FIG. 10 is a graph showing average IFNα2a serum concentration over time as determined by ELISA and Anti-Viral Assay (AVA) for the formulations referenced in FIGS. 8 and 9.

FIG. 11 is a graph showing average serum concentration over time for a nucleoside analogue pro-drug delivered in primate.

FIG. 12 is a graph showing average serum concentration over time for the active metabolite of the nucleoside analogue pro-drug of FIG. 11.

FIG. 13 is a graph showing equivalent dose plasma profiles for a Glucagon-like peptide-1 (GLP-1) analogue delivered in mini-pig.

FIG. 14 provides graphs showing average serum profiles in rats for rhGH delivered from depots containing free protein dispersed in various BB:Polymer (80:20) vehicles (A), and delivered from depots containing rhGH:Protamine complex dispersed in various BB:Polymer (80:20) vehicles (B).

FIG. 15 (Panels A-E) provides graphs which show within formulation comparisons of serum profiles with free vs. complexed rhGH for the formulations shown in FIG. 14.

FIG. 16 provides graphs showing the results for three rhGH complexes tested in vehicles containing either lactate-initiated PLA, 15.1 kDa, or dodecanol-initiated PLA, 13.9 kDa and compared with uncomplexed (free) rhGH formulations. (A) All forms of rhGH in BB, (B) All forms of rhGH in BB:lactate-initiated-PLA 80:20, (C) All forms of rhGH in BB:dodecanol-initiated PLA 80:20.

FIG. 17 provides a graph showing average mean residence times (MRTs) for each formulation described in FIG. 16.

FIG. 18 shows the fractional contribution of polymer-complex interaction to MRT for Examples 11 and 12.

FIG. 19 provides a photograph of the initiation of cloud formation in a SAIB/BB/PLA vehicle. A 23 G regular needle was used to inject approximately 0.5 mL of a SAIB/BB/PLA (LA-initiated) (8:72:20) vehicle into PBS buffer at pH 7.4 and 37° C. A first picture was taken at about 10 sec following initiation of injection.

FIG. 20 provides a second photograph of the vehicle depicted in FIG. 19 taken about 60 seconds following the completion of the 0.5 mL injection.

FIG. 21 provides a graph showing the viscosity stability of cloud forming vehicle formulations over time at 37° C. Viscosity is characterized for the following vehicle formulations: SAIB/BB/PLA (8/72/20), SAIB/BB/BA/PLA (20/60/10/10), SAIB/BB/EtOH/PLGA 65:35 (8/67/5/20), BB/BA/PLA (70/10/20).

FIG. 22 provides a graph showing viscosity stability as a function of temperature for the vehicle formulations described in FIG. 21.

FIG. 23 provides a graph showing the average serum concentration over time for each of the treatment conditions identified in Examples 19 and 20.

FIG. 24 provides a graph showing mean dose-normalized rhGH serum profiles for BA:dd-PLGA and BA:ga-PLGA vehicles.

FIG. 25 provides a graph showing mean dose-normalized rhGH serum profiles for EB:dd-PLGA and EB:ga-PLGA vehicles.

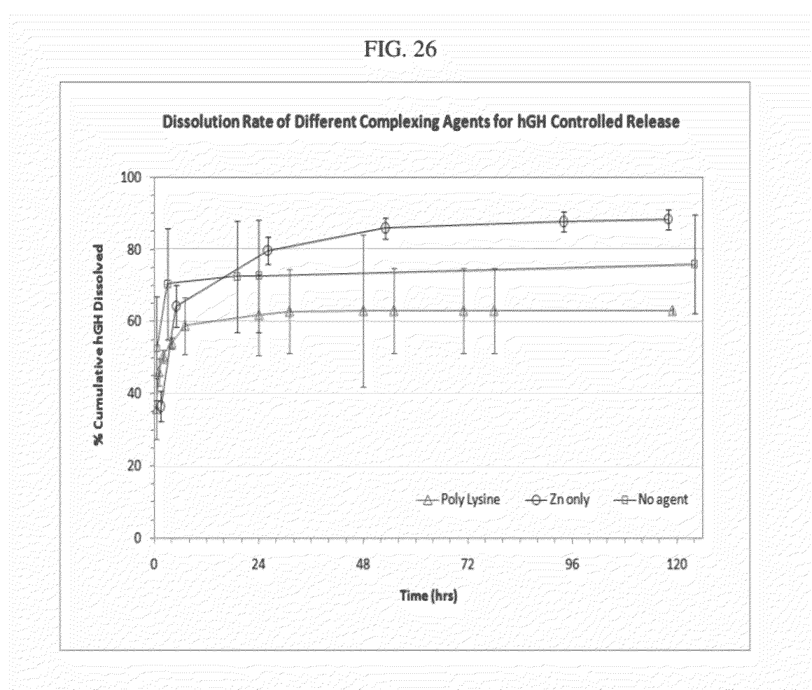

FIGS. 26 and 27 provide graphs showing dissolution rate of hGH from different complexing agents for the controlled delivery of hGH up to 5 days.

FIG. 28 provides a graph showing % cumulative dissolution over time for various hGH powder formulations.

FIG. 29 provides a graph showing serum concentration over time for a peptide beneficial agent (Exenatide) in the following formulations: Exenatide:protamine 1:2 (m/m), lyophilized, 9.5 mg dose, in SAIB/BB/la-PLA (8/72/20) and Exenatide:protamine 1:2 (m/m), spray dried, 9.5 mg dose, SAIB/BB/la-PLA (8/72/20) methionine & polysorbate 80.

FIG. 30 provides a depiction of one embodiment of a composition according to the present disclosure including a charge-neutralized peptide or protein beneficial agent complex including Zn²⁺ and protamine.

### DEFINITIONS

As used herein, the term “insoluble component” refers to a component of a composition as described herein which includes an insoluble beneficial agent and/or an insoluble beneficial agent complex as defined herein.

As used herein, the term “insoluble beneficial agent” refers to a beneficial agent which is completely or substantially insoluble. The term “substantially insoluble” as used in this context means that at least 90%, e.g., at least 95%, at least 98%, at least 99%, or at least 99.5% of the beneficial agent is insoluble in the vehicle at 25° C. In other words, an insoluble beneficial agent is a beneficial agent which may be dispersed in a vehicle and which is not significantly dissolved in the vehicle. An insoluble beneficial agent may include, e.g., a molecule which is substantially insoluble in a vehicle composition as described herein. An insoluble beneficial agent may include, for example, a beneficial agent having a solubility of less than 1 mg/mL in the vehicle at 25° C.

As used herein, the term “insoluble beneficial agent complex” refers to beneficial agent complexes which are completely or substantially insoluble in the vehicle. The term “substantially insoluble” as used in this context means that at least 90%, e.g., at least 95%, at least 98%, at least 99%, or at least 99.5% of the beneficial agent complex is insoluble in the vehicle at 25° C. For instance, an insoluble beneficial agent complex is a complex which may be dispersed in a vehicle and which is not significantly dissolved in the vehicle. An insoluble beneficial agent complex may include, e.g., a charge-neutralized complex. An insoluble beneficial agent complex may include, for example, a beneficial agent having a solubility of less than 1 mg/mL in the vehicle at 25° C.

The term “charge-neutralized complex” is used herein to refer to a complex formed as a result of a non-covalent charge-based interaction between a beneficial agent and an associated molecule, metal, counter ion, etc., and having no net charge or substantially no net charge. Included within this definition are charge neutralized beneficial agents including salts of the beneficial agents.

As used herein, the term “vehicle” means a composition including a biodegradable polymer and a hydrophobic solvent in the absence of a beneficial agent as described herein.

As used herein, the term “zero shear viscosity” means viscosity at zero shear rate. A skilled artisan would be able to determine zero shear viscosity by measuring viscosity at low shear rate (e.g., around 1 sec⁻¹ to 7 sec⁻¹) using a plate and cone viscometer (e.g., Brookfield Model DV-III+(LV)) and then extrapolating a plot of viscosity versus shear rate to a shear rate of zero at a temperature of interest.

As used herein, the term “emulsion” means a stable mixture of two or more immiscible liquids, including a continuous phase and a dispersed phase.

As used herein, the term “emulsifying agent” means an agent which when included in a biodegradable composition as described herein tends to form an emulsion.

As used herein, the term “beneficial agent” means an agent, e.g., a protein, peptide, nucleic acid (including nucleotides, nucleosides and analogues thereof) or small molecule drug, that provides a desired pharmacological effect upon administration to a subject, e.g., a human or a non-human animal, either alone or in combination with other active or inert components. Included in the above definition are precursors, derivatives, analogues and prodrugs of beneficial agents.

As used herein, the term “non-aqueous” refers to a substance that is substantially free of water. Non-aqueous compositions have a water content of less than about 5%, such as less than about 2%, less than about 1%, less than 0.5%, or less than 0.1%, by weight. The present compositions are typically non-aqueous.

As used herein, the terms “burst effect” and “burst” are used interchangeably to mean a rapid, initial release of beneficial agent from a composition following administration of the composition which may be distinguished from a subsequent relatively stable, controlled period of release.

As used herein the term “syringeability” describes the ability of a composition to pass easily through a hypodermic needle on transfer from a container prior to injection. Syringeability may be quantified, for example, by measuring the force required to move a known amount of a composition through a syringe and needle, per unit time.

As used herein the term “injectability” refers to the performance of a composition during injection and includes factors such as pressure or force required for injection, evenness of flow, aspiration qualities, and freedom from clogging. Injectability may be quantified e.g., by measuring the force required to move a known amount of a composition through a syringe and needle, per unit time.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “nucleic acid,” “nucleic acid molecule”, “oligonucleotide” and “polynucleotide” are used interchangeably and refer to a polymeric form of nucleotides of any length, either deoxyribonucleotides or ribonucleotides, or compounds produced synthetically which can hybridize with naturally occurring nucleic acids in a sequence specific manner similar to that of two naturally occurring nucleic acids, e.g., can participate in Watson-Crick base pairing interactions. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, cDNA, recombinant polynucleotides, plasmids, vectors, isolated DNA of any sequence, control regions, isolated RNA of any sequence, nucleic acid probes, and primers.

The terms “rate controlling cloud,” “rate controlling film,” and “rate controlling surface layer” are used interchangeably herein to refer to a rate controlling element of a formulation which is formed at the formulation surface and an aqueous environment, which surrounds a substantially liquid core and has a release rate-controlling effect on a beneficial agent from the substantially liquid core of the formulation to the aqueous environment. Unlike polymeric matrices that are formed by a phase inversion, phase separation, or gelation process in an aqueous environment, the rate controlling cloud or film does not have appreciable physical strength or mechanical structure.

As used herein “bioavailability” refers to the fraction of the beneficial agent dose that enters the systemic circulation following administration.

As used herein “mean residence time (MRT)” refers to the average total time molecules of a given dose reside in the body which may be calculated as area under the first moment curve (AUMC)/area under the curve (AUC), where

AUC=∫₀^{∞}Cₚ(t)dt

and

AUMC=∫₀^{∞}Cₚ(t)·tdt

and, where Cₚ(t) is plasma (or serum or blood) concentration as a function of time.

As used herein, the term “gel” refers to a composition which has a relatively small G″/G′ ratio, for example less than or equal to one, wherein G″=the loss modulus and G′=the storage modulus. Conversely, the terms “non-gel”, “not a gel” and the like refer to a composition which has a relatively large G″/G′ ratio, e.g., a G″/G′ ratio of greater than or equal to 10.

As used herein, the terms “gelling”, “gel-forming” and the like refer to a composition which has a relatively small G″/G′ ratio, for example less than or equal to one (e.g., following aging at 37° C. for a period of 14 days), wherein G″=the loss modulus and G′=the storage modulus. Conversely, the terms “non-gelling”, “non-gel forming” and the like are used herein to refer to a composition which has a relatively large G″/G′ ratio, e.g., a G″/G′ ratio of greater than or equal to 10 (e.g., following aging at 37° C. for a period of 14 days).

As used herein “physical stability” refers to the ability of a material, e.g., a compound or complex to resist physical change.

As used herein “chemical stability” refers to the ability of a material, e.g., a compound or complex to resist chemical change.

As used herein, the terms “Glucagon-like-peptide-1” and “GLP-1” refer to a molecule having GLP-1 activity. One of ordinary skill in the art can determine whether any given moiety has GLP-1 activity, as disclosed in U.S. Published Application No. 2010/0210505, which is incorporated herein by reference. The term “GLP-1” includes native GLP-1 (GLP-1 (7-37)OH or GLP-1 (7-36)NH₂), GLP-1 analogs, GLP-1 derivatives, GLP-1 biologically active fragments, extended GLP-1 (see, for example, International Patent Publication No. WO 03/058203, which is incorporated herein by reference, in particular with respect to the extended glucagon-like peptide-1 analogs described therein), exendin-4, exendin-4 analogs, and exendin-4 derivatives comprising one or two cysteine residues at particular positions as described in WO 2004/093823, which is incorporated herein by reference.

When used to characterize a vehicle component or components as described herein, the term “% w/w” refers to % by weight of the vehicle, for example, SAIB/BB/PLA (8:72:20, % w/w) identifies a vehicle including SAIB at 8% by weight of the vehicle, BB at 72% by weight of the vehicle, and PLA at 20% by weight of the vehicle.

Before the present invention is further described, it is to be understood that this invention is not limited to particular embodiments described, as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an insoluble beneficial agent complex” includes a plurality of such complexes and reference to “the injectable depot composition” includes reference to one or more injectable depot compositions and equivalents thereof, and so forth. It is further noted that the claims may be drafted to exclude any optional element. As such, this statement is intended to provide antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

As discussed above, the present disclosure provides a biodegradable drug delivery composition, e.g., an injectable biodegradable drug delivery depot composition, including a vehicle, e.g., a single phase vehicle, and an insoluble component comprising beneficial agent, e.g., an insoluble beneficial agent complex, dispersed in the vehicle. In some embodiments, the vehicle includes a biodegradable polymer present in an amount of from about 5% to about 40% by weight of the vehicle and a hydrophobic solvent (or mixture of hydrophobic solvents) present in an amount of from about 95% to about 60% by weight of the vehicle. In addition to the vehicle, the composition includes an insoluble component comprising beneficial agent, e.g., an insoluble beneficial agent complex, dispersed in the vehicle. In some embodiments, the biodegradable composition has a zero shear viscosity less than 1,200 centipoise at 25° C. and is not an emulsion or gel.

### Biocompatible-Biodegradable Polymers

A variety of polymers may be suitable for use in the compositions of the present disclosure provided that they are both biocompatible and biodegradable. For example, suitable polymers may include, but are not limited to, homopolymers, block-copolymers and random copolymers. Suitable polymers include those polymers or combinations of polymers which have solubility of at least about 20 weight %, 30 weight %, or 40 weight % in the selected solvent or solvent combination. In some embodiments, suitable polymers include polymers having both hydrophilic and hydrophobic regions, e.g., an AB-type block copolymer composed of hydrophobic and hydrophilic components. Such polymers may have a tendency to form micelles when exposed to an aqueous environment as a result of the amphiphilic character of the polymer. Suitable polymers may include, but are not limited to, polylactides, polyglycolides, polycaprolactones, copolymers including any combination of two or more monomers involved in the above, e.g., terpolymers of lactide, glycolide and c-caprolactone, and mixtures including any combination of two or more of the above. In other words, suitable polymers may also include, for example, polylactic acids, polyglycolic acids, polycaprolactones, copolymers including any combination of two or more monomers involved in the above, e.g., terpolymers of lactic acid, glycolic acid and c-caprolactone, and mixtures including any combination of two or more of the above.

In some embodiments, the biodegradable polymer is polylactic acid (PLA), e.g., a PLA including an ionizable end-group (e.g., an acid end-group, e.g., in an acid-terminated PLA). Acid end-group PLAs include, e.g., lactate initiated PLAs described herein. In some embodiments, the PLA includes an unionizable end-group (e.g., an ester end-group, e.g., in an ester terminated PLA). Ester end-group PLAs include, but are not limited to, dodecanol-initiated (dd) PLAs described herein. In some embodiments, the PLA is dl-PLA. In other embodiments, the biodegradable polymer is poly(lactic-co-glycolic acid) (PLGA), e.g., dl-PLGA. In some embodiments, the PLGA includes an ionizable end-group, e.g., an acid end-group. Acid end-group PLGAs include, but are not limited to, the glycolate initiated (ga) PLGAs described herein. In some embodiments, the PLGA includes an unionizable end-group, e.g., an ester end group. Ester end-group PLGAs include, but are not limited to, dodecanol initiated PLGAs described herein. In one embodiment, where the polymer is a polycaprolactone, the polycaprolactone is poly(ε)caprolactone.

The biocompatible, biodegradable polymer is present in the vehicle in an amount ranging from about 5% to about 40% by weight of the vehicle, for example, from about 6% to about 35%, from about 7% to about 30%, from about 8% to about 27%, from about 9% to about 26%, from about 10% to about 25%, from about 11% to about 24%, from about 12% to about 23%, from about 13% to about 22%, from about 14% to about 21%, from about 15% to about 20%, from about 16% to about 19%, or at about 17% by weight of the vehicle. In some embodiments, the polymer is present in an amount of about 20% by weight of the vehicle.

In some embodiments, the biocompatible, biodegradable polymer has a weight average molecular weight of from about 2 kD to about 20 kD, e.g., from about 2 kD to about 5 kD, from about 2 kD to about 10 kD, or from about 2 kD to about 15 kD. Additional embodiments include a biocompatible, biodegradable polymer having a weight average molecular weight of from about 5 kD to about 15 kD, e.g., about 10 kD.

### Solvents

Hydrophobic solvents suitable for use in the compositions of the present disclosure are hydrophobic solvents which are capable of solubilizing a polymer component of the vehicles described herein. Hydrophobic solvents can be characterized as being insoluble or substantially insoluble in water. For example, suitable hydrophobic solvents have solubility in water of less than 5% by weight, less than 4% by weight, less than 3% by weight, less than 2% by weight or less than 1% by weight, e.g. as measured at 25° C. A suitable hydrophobic solvent may also be characterized as one which has a solubility in water of about 5% or less, about 4% or less, about 3% or less, about 2% or less, or about 1% or less, at 25° C. For example, in some embodiments, a suitable hydrophobic solvent has a solubility in water of from about 1% to about 7%, from about 1% to about 6%, from about 1% to about 5%, from about 1% to about 4%, from about 1% to about 3%, and from about 1% to about 2%, at 25° C. A suitable hydrophobic solvent may also be characterized as a solvent in which water has limited solubility, e.g., a solvent in which water has solubility of less than 10% by weight, less than 5% by weight, or less than 1% by weight, at 25° C. In some embodiments, a suitable hydrophobic solvent is one which solubilizes the polymer component of the vehicle and which when combined with the polymer component in a suitable amount as described herein results in a vehicle having a low viscosity, i.e., a zero shear viscosity less than 1,200 centipoise at 25° C.

In some embodiments, suitable solvents include derivatives of benzoic acid including, but not limited to, benzyl alcohol, methyl benzoate, ethyl benzoate, n-propyl benzoate, isopropyl benzoate, butyl benzoate, isobutyl benzoate, sec-butyl benzoate, tert-butyl benzoate, isoamyl benzoate and benzyl benzoate.

In some embodiments, benzyl benzoate is selected as the hydrophobic solvent for use in the biodegradable delivery compositions of the present disclosure.

A suitable solvent may be a single solvent selected from among the following or a combination of two or more of the following: benzyl alcohol, benzyl benzoate, ethyl benzoate, and ethanol.

Where the solvent is a hydrophobic solvent, it may be used in combination with one or more additional solvents, e.g., one or more hydrophobic solvents and/or one or more polar/hydrophilic solvents.

In some embodiments, the compositions include a single hydrophobic solvent as described herein without including any additional solvents. In some embodiments, the single hydrophobic solvent is benzyl benzoate, in other embodiments the single hydrophobic solvent is other than benzyl alcohol.

Where the solvent is a polar/hydrophilic solvent, it is used in the disclosed compositions only in combination with a hydrophobic solvent and is present in a relatively small amount relative to the hydrophobic solvent, e.g., less than 5% (e.g., less than 4%, less than 3%, less than 2%, or less than 1%) by weight of the vehicle. For example, a polar/hydrophilic solvent may be present in the vehicle in an amount of from about 5% to about 1% (e.g., from about 4% to about 1%, from about 3% to about 1%, or from about 2% to about 1%) by weight of the vehicle. Without wishing to be bound by any particular theory, it is believed that the addition of relatively small amounts of polar/hydrophilic solvent, e.g., ethanol, to the vehicle composition may broaden the range of polymers in terms of polymer type, molecular weight, and relative hydrophobicity/hydrophilicity which may be utilized in the disclosed compositions.

The hydrophobic solvent (or combination of hydrophobic solvents) is present in the vehicle from about 95% to about 60% by weight of the vehicle, for example, from about 94% to about 61%, from about 93% to about 62%, from about 92% to about 63%, from about 91% to about 64%, from about 90% to about 65%, from about 89% to about 66%, from about 88% to about 67%, from about 87% to about 68%, from about 86% to about 69%, from about 85% to about 70%, from about 84% to about 71%, from about 83% to about 72%, from about 82% to about 73%, from about 81% to about 74%, from about 80% to about 75%, from about 79% to about 76%, or from about 78% to about 77% by weight of the vehicle. In some embodiments, the hydrophobic solvent (or combination of hydrophobic solvents) is present in the vehicle from about 95% to about 90%, from about 95% to about 85%, from about 95% to about 80%, from about 95% to about 75%, from about 95% to about 70%, from about 95% to about 65%, or from about 95% to about 60% by weight of the vehicle. In some embodiments, the hydrophobic solvent is present in an amount of about 80% by weight of the vehicle. In other embodiments, the hydrophobic solvent is present in an amount of about 72% by weight of the vehicle.

In some embodiments, the biodegradable drug delivery compositions disclosed herein are free of hydrophilic solvent. In some embodiments, the biodegradable delivery compositions disclosed herein do not include a thixotropic agent, e.g., a lower alkanol containing 2-6 carbon atoms.

### Beneficial Agents

A variety of beneficial agents may be delivered using the biodegradable delivery compositions disclosed herein. General classes of beneficial agents which may be delivered include, for example, proteins, peptides, nucleic acids, nucleotides, nucleosides and analogues thereof, antigens, antibodies, and vaccines; as well as low molecular weight compounds.

In some embodiments, the beneficial agent is at least substantially insoluble in the vehicle, e.g., solubility in the vehicle less than 10 mg/mL, less than 5 mg/mL, less than 1 mg/mL, less than 0.5 mg/mL, less than 0.3 mg/mL, less than 0.2 mg/mL, or less than 0.1 mg/mL.

Beneficial agents which may be delivered using the biodegradable delivery compositions disclosed herein include, but are not limited to, agents which act on the peripheral nerves, adrenergic receptors, cholinergic receptors, the skeletal muscles, the cardiovascular system, smooth muscles, the blood circulatory system, synaptic sites, neuroeffector junction sites, endocrine and hormone systems, the immunological system, the reproductive system, the skeletal system, autacoid systems, the alimentary and excretory systems, the histamine system and the central nervous system.

Suitable beneficial agents may be selected, for example, from chemotherapeutic agents, epigenetic agents, proteasome inhibitors, adjuvant drugs, anti-emetics, appetite stimulants, anti-wasting agents and high potency opioids.

Suitable beneficial agents may also be selected, for example, from anti-neoplastic agents, cardiovascular agents, renal agents, gastrointestinal agents, rheumatologic agents and neurological agents among others.

Protein, Polypeptides and Peptides as Beneficial Agents

Proteins useful in the disclosed formulations may include, for example, molecules such as cytokines and their receptors, as well as chimeric proteins comprising cytokines or their receptors, including, for example tumor necrosis factor alpha and beta, their receptors and their derivatives; renin; growth hormones, including human growth hormone, bovine growth hormone, methione-human growth hormone, des-phenylalanine human growth hormone, and porcine growth hormone; growth hormone releasing factor (GRF); parathyroid and pituitary hormones; thyroid stimulating hormone; human pancreas hormone releasing factor; lipoproteins; colchicine; prolactin; corticotrophin; thyrotropic hormone; oxytocin; vasopressin; somatostatin; lypressin; pancreozymin; leuprolide; alpha-1-antitrypsin; insulin A-chain; insulin B-chain; proinsulin; follicle stimulating hormone; calcitonin; luteinizing hormone; luteinizing hormone releasing hormone (LHRH); LHRH agonists and antagonists; glucagon; clotting factors such as factor VIIIC, factor IX, tissue factor, and von Willebrands factor; anti-clotting factors such as Protein C; atrial natriuretic factor; lung surfactant; a plasminogen activator other than a tissue-type plasminogen activator (t-PA), for example a urokinase; bombesin; thrombin; hemopoietic growth factor; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; chorionic gonadotropin; gonadotropin releasing hormone; bovine somatotropin; porcine somatotropin; a microbial protein, such as beta-lactamase; DNase; inhibin; activin; vascular endothelial growth factor (VEGF); receptors for hormones or growth factors; integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as acidic FGF and basic FGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-alpha (e.g., interferonβ2A or interferonα2B), -beta, -gamma, -lambda and consensus interferon; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the HIV-1 envelope glycoprotein, gp120, gp160 or fragments thereof; transport proteins; homing receptors; addressins; fertility inhibitors such as the prostaglandins; fertility promoters; regulatory proteins; antibodies and chimeric proteins, such as immunoadhesins; precursors, derivatives, prodrugs and analogues of these compounds, and pharmaceutically acceptable salts of these compounds, or their precursors, derivatives, prodrugs and analogues.

Suitable proteins or peptides may be native or recombinant and include, e.g., fusion proteins.

In some embodiments, the protein is a growth hormone, such as human growth hormone (hGH), recombinant human growth hormone (rhGH), bovine growth hormone, methione-human growth hormone, des-phenylalanine human growth hormone, and porcine growth hormone; insulin, insulin A-chain, insulin B-chain, and proinsulin; or a growth factor, such as vascular endothelial growth factor (VEGF), nerve growth factor (NGF), platelet-derived growth factor (PDGF), fibroblast growth factor (FGF), epidermal growth factor (EGF), transforming growth factor (TGF), and insulin-like growth factor-I and -II (IGF-I and IGF-II).

Suitable peptides for use as the beneficial agent in the biodegradable delivery compositions disclosed herein include, but are not limited to, Glucagon-like peptide-1 (GLP-1) and precursors, derivatives, prodrugs and analogues thereof.

In addition, a suitable protein, polypeptide, peptide; or precursor, derivative, prodrug or analogue thereof is one which is capable of forming an insoluble component comprising beneficial agent, e.g., an insoluble beneficial agent complex, e.g., by complexing with a metal or other precipitating and/or stabilizing agent as described herein.

In some embodiments, the beneficial agent comprises growth hormone and the hydrophobic solvent does not comprise benzyl alcohol. In some embodiments, the beneficial agent comprises growth hormone and the hydrophobic solvent does not comprise ethyl benzoate.

Nucleic Acids as Beneficial Agents

Nucleic acid beneficial agents include nucleic acids as well as precursors, derivatives, prodrugs and analogues thereof, e.g., therapeutic nucleotides, nucleosides and analogues thereof; therapeutic oligonucleotides; and therapeutic polynucleotides. Beneficial agents selected from this group may find particular use as anticancer agents and antivirals. Suitable nucleic acid beneficial agents may include for example ribozymes, antisense oligodeoxynucleotides, aptamers and siRNA. Examples of suitable nucleoside analogues include, but are not limited to, cytarabine (araCTP), gemcitabine (dFdCTP), and floxuridine (FdUTP).

Other Beneficial Agent Compounds

A variety of other beneficial agent compounds may be used in the compositions disclosed herein. Suitable compounds may include, but are not limited to, compounds directed to one or more of the following drug targets: Kringle domain, Carboxypeptidase, Carboxylic ester hydrolases, Glycosylases, Rhodopsin-like dopamine receptors, Rhodopsin-like adrenoceptors, Rhodopsin-like histamine receptors, Rhodopsin-like serotonin receptors, Rhodopsin-like short peptide receptors, Rhodopsin-like acetylcholine receptors, Rhodopsin-like nucleotide-like receptors, Rhodopsin-like lipid-like ligand receptors, Rhodopsin-like melatonin receptors, Metalloprotease, Transporter ATPase, Carboxylic ester hydrolases, Peroxidase, Lipoxygenase, DOPA decarboxylase, A/G cyclase, Methyltransferases, Sulphonylurea receptors, other transporters (e.g., Dopamine transporter, GABA transporter 1, Norepinephrine transporter, Potassium-transporting ATPase α-chain 1, Sodium-(potassium)-chloride cotransporter 2, Serotonin transporter, Synaptic vesicular amine transporter, and Thiazide-sensitive sodium-chloride cotransporter), Electrochemical nucleoside transporter, Voltage-gated ion channels, GABA receptors (Cys-Loop), Acetylcholine receptors (Cys-Loop), NMDA receptors, 5-HT3 receptors (Cys-Loop), Ligand-gated ion channels Glu: kainite, AMPA Glu receptors, Acid-sensing ion channels aldosterone, Ryanodine receptors, Vitamin K⁺ epoxide reductase, MetGluR-like GABA_{B} receptors, Inwardly rectifying K⁺ channel, NPC1L1, MetGluR-like calcium-sensing receptors, Aldehyde dehydrogenases, Tyrosine 3-hydroxylase, Aldose reductase, Xanthine dehydrogenase, Ribonucleoside reductase, Dihydrofolate reductase, IMP dehydrogenase, Thioredoxin reductase, Dioxygenase, Inositol monophosphatase, Phosphodiesterases, Adenosine deaminase, Peptidylprolyl isomerases, Thymidylate synthase, Aminotransferases, Farnesyl diphosphate synthase, Protein kinases, Carbonic anhydrase, Tubulins, Troponin, Inhibitor of IκB kinase-β, Amine oxidases, Cyclooxygenases, Cytochrome P450s, Thyroxine 5-deiodinase, Steroid dehydrogenase, HMG-CoA reductase, Steroid reductases, Dihydroorotate oxidase, Epoxide hydrolase, Transporter ATPase, Translocator, Glycosyltransferases, Nuclear receptors NR3 receptors, Nuclear receptors: NR1 receptors, and Topoisomerase.

In some embodiments, the beneficial agent is a compound targeting one of rhodopsin-like GPCRs, nuclear receptors, ligand-gated ion channels, voltage-gated ion channels, penicillin-binding protein, myeloperoxidase-like, sodium: neurotransmitter symporter family, type II DNA topoisomerase, fibronectin type III, and cytochrome P450.

In some embodiments, the beneficial agent is an anticancer agent. Suitable anticancer agents include, but are not limited to, Actinomycin D, Alemtuzumab, Allopurinol sodium, Amifostine, Amsacrine, Anastrozole, Ara-CMP, Asparaginase, Azacytadine, Bendamustine, Bevacizumab, Bicalutimide, Bleomycin (e.g., Bleomycin A₂ and B₂), Bortezomib, Busulfan, Camptothecin sodium salt, Capecitabine, Carboplatin, Carmustine, Cetuximab, Chlorambucil, Cisplatin, Cladribine, Clofarabine, Cyclophosphamide, Cytarabine, Dacarbazine, Dactinomycin, Daunorubicin, Daunorubicin liposomal, Dacarbazine, Decitabine, Docetaxel, Doxorubicin, Doxorubicin liposomal, Epirubicin, Estramustine, Etoposide, Etoposide phosphate, Exemestane, Floxuridine, Fludarabine, Fluadarabine phosphate, 5-Fluorouracil, Fotemustine, Fulvestrant, Gemcitabine, Goserelin, Hexamethylmelamine, Hydroxyurea, Idarubicin, Ifosfamide, Imatinib, Irinotecan, Ixabepilone, Lapatinib, Letrozole, Leuprolide acetate, Lomustine, Mechlorethamine, Melphalan, 6-Mercaptopurine, Methotrexate, Mithramycin, Mitomycin C, Mitotane, Mitoxantrone, Nimustine, Ofatumumab, Oxaliplatin, Paclitaxel, Panitumumab, Pegaspargase, Pemetrexed, Pentostatin, Pertuzumab, Picoplatin, Pipobroman, Plerixafor, Procarbazine, Raltitrexed, Rituximab, Streptozocin, Temozolomide, Teniposide, 6-Thioguanine, Thiotepa, Topotecan, Trastuzumab, Treosulfan, Triethylenemelamine, Trimetrexate, Uracil Nitrogen Mustard, Valrubicin, Vinblastine, Vincristine, Vindesine, Vinorelbine, and analogues, precursors, derivatives and pro-drugs thereof. It should be noted that two or more of the above compounds may be used in combination in the compositions of the present disclosure.

Beneficial agents of interest for use in the disclosed compositions may also include opioids and derivatives thereof as well as opioid receptor agonists and antagonists, e.g., methadone, naltrexone, naloxone, nalbuphine, fentanyl, sufentanil, oxycodone, oxymorphone, hydrocodone, hydromorphone, and pharmaceutically acceptable salts and derivatives thereof.

In some embodiments the beneficial agent is a low molecular weight compound, e.g., a compound having a molecular weight of less than or equal to about 800 Daltons. In some embodiments, where the beneficial agent is a low molecular weight compound, the beneficial agent is one which has solubility in water of 10 to 100 mg/ml or less, e.g., less than 100 mg/ml, less than 90 mg/ml, less than 80 mg/ml, less than 70 mg/ml, less than 60 mg/ml, less than 50 mg/ml, less than 40 mg/ml, less than 30 mg/ml, less than 20 mg/ml, less than 10 mg/ml, less than 5 mg/ml, or less than 1 mg/ml.

In some embodiments, a low molecular weight compound suitable for use as a beneficial agent is a compound that is at least substantially insoluble in the vehicle, e.g., solubility in the vehicle is less than 10 mg/mL, less than 5 mg/mL, less than 1 mg/mL, less than 0.5 mg/mL, less than 0.3 mg/mL, less than 0.2 mg/mL, or less than 0.1 mg/mL.

In some embodiments, a low molecular weight compound suitable for use as a beneficial agent is a compound which when present in salt form is at least substantially insoluble in the vehicle, e.g., solubility in the vehicle is less than 10 mg/mL, less than 5 mg/mL, less than 1 mg/mL, less than 0.5 mg/mL, less than 0.3 mg/mL, less than 0.2 mg/mL, or less than 0.1 mg/mL.

The beneficial agent or beneficial agent complex may be present in any suitable concentration in the biodegradable compositions disclosed herein. Suitable concentrations may vary depending on the potency of the beneficial agent, beneficial agent pharmacokinetic half-life, etc. For example, the insoluble component comprising beneficial agent, e.g., insoluble beneficial agent complex, may be present in a range of from about 1% to about 50% by weight of the composition, e.g., from about 5% to about 45%, from about 10% to about 40%, from about 15% to about 35%, or from about 20% to about 30% by weight of the composition. The insoluble component comprising beneficial agent, e.g., insoluble beneficial agent complex, may be present at a concentration ranging from about 10 mg/mL to about 500 mg/mL, such as from about 50 mg/mL to about 450 mg/mL, about 100 mg/mL to about 400 mg/mL, about 150 mg/mL to about 350 mg/mL, or about 200 mg/mL to about 300 mg/mL.

In some embodiments, the beneficial agent is an insoluble beneficial agent as defined herein, i.e., a beneficial agent which is completely or substantially insoluble in the vehicle chosen for use in connection with the biodegradable drug delivery compositions described herein. In other words, at least 90%, e.g., at least 95%, at least 98%, at least 99%, or at least 99.5% of the beneficial agent is insoluble in the vehicle at 25° C. An insoluble beneficial agent is a beneficial agent which may be dispersed in a vehicle and which is not significantly dissolved in the vehicle. An insoluble beneficial agent may include, e.g., a molecule which is substantially insoluble in a vehicle composition as described herein.

### Insoluble Complex

The beneficial agent may be provided as an insoluble beneficial agent complex, e.g., an electrostatic complex, which is dispersed in the vehicle. Complexing may be used to reduce the solubility of beneficial agents. As defined previously herein, the term “insoluble beneficial agent complex”, includes beneficial agent complexes which are completely or substantially insoluble in the vehicle chosen for use in connection with the biodegradable drug delivery compositions described herein. The term “substantially insoluble” as used in this context means that at least 90%, e.g., at least 95%, at least 98%, at least 99%, or at least 99.5%, of the beneficial agent complex is insoluble in the vehicle at 25° C. In other words, an insoluble beneficial agent complex is a complex which may be dispersed in a vehicle and which is not significantly dissolved in the vehicle. An insoluble beneficial agent complex may include, e.g., a charge-neutralized complex. The term “charge-neutralized complex” is used herein to refer to a complex formed as a result of a non-covalent charge-based interaction between a beneficial agent and an associated molecule, metal, counter ion, etc., and having no net charge or substantially no net charge. Included within this definition are charge neutralized beneficial agents including salts of the beneficial agents.

This complexation contributes to the beneficial release characteristics of the disclosed compositions as discussed herein, e.g., by contributing to the chemical and physical stability of the beneficial agent in the composition, e.g., by reducing degradation of the beneficial agent or providing a complex, which exhibits reduced settling due to gravitational force. In some embodiments, the insoluble beneficial agent complex is formed by including a precipitating and/or stabilizing agent which when combined with the beneficial agent induces formation of an insoluble complex. The insoluble beneficial agent complex may result, for example, from an electrostatic interaction which takes place between the beneficial agent and one or more precipitating and/or stabilizing agents. In some embodiments, the insoluble beneficial agent complex is charge neutralized. Complexation may also reduce a level of chemical conjugation which may occur between the beneficial agent and other components of the formulation, e.g., polymer, in the absence of the complexation.

The insoluble beneficial agent complex according to the present disclosure may be characterized as follows: when 10 mg of the insoluble beneficial agent complex is dispersed and left to stand in 1 mL of a test solution of phosphate buffered saline at pH 7.4 at 37° C. for 24 hours, the amount of beneficial agent dissolved in the test solution is less than 60% of the beneficial agent in the 10 mg of insoluble beneficial agent complex, e.g., less than 50% of the beneficial agent in the 5 mg of insoluble beneficial agent complex, less than 40% of the beneficial agent in the 5 mg of insoluble beneficial agent complex, less than 30% of the beneficial agent in the 5 mg of insoluble beneficial agent complex, or less than 20% of the beneficial agent in the 5 mg of insoluble beneficial agent complex.

In some embodiments, the precipitating or stabilizing agent is a charged species, e.g. a charged molecule, a metal ion or a salt form of a metal ion. Persons having ordinary skill in the art will understand that the salt forms of metal ions are not themselves charged species, but rather provide the source, upon dissociation, of the charged species. For example, in some embodiments, the precipitating agent and/or stabilizing agent is protamine, or a divalent metal ion such as Ni²⁺, Cu²⁺, Zn²⁺, Mg²⁺ and/or Ca²⁺. The divalent metal may be present in the composition as e.g., zinc acetate, zinc carbonate, zinc chloride, zinc sulfate, magnesium acetate, magnesium carbonate, magnesium chloride, magnesium hydroxide, magnesium oxide, magnesium sulfate, calcium acetate, calcium carbonate, calcium chloride, calcium sulfate and the like. That is, the divalent metal salt may be included during preparation of the composition such that a divalent metal salt of the beneficial agent is formed. These precipitating agents and/or stabilizing agents find particular use when the selected beneficial agent is a negatively charged protein or peptide.

It should be noted that the net charge of the beneficial agent may also be adjusted, for example by adjusting the pH. Accordingly, a suitably charged precipitating agent and/or stabilizing agent may be selected based on the net charge of the protein or peptide which may be adjusted. For example, where the beneficial agent has a net positive charge, e.g., as a result of pH adjustment, a negatively charged molecule such as carboxymethylcellulose (CMC) may be utilized as the precipitating agent and/or stabilizing agent.

Thus, some embodiments involve a method of making a complex involving contacting at least one of a protein and peptide with a cationic complexing agent at a pH greater than 8, e.g., greater than 8.5 or greater than 9, such as 8 to 10, or 8 to 9, to form a complex. Examples of the cationic complexing agent include, but are not limited to, protamine, poly-lysine, poly-arginine, polymyxin, and combinations thereof.

Other embodiments involve a method of making a complex involving contacting at least one of a protein and peptide with an anionic complexing agent at a pH less than 3, e.g., less than 2.5 or less than 2, such as 1 to 3 or 2 to 3, to form a complex. Examples of the anionic complexing agent include, but are not limited to, carboxy-methyl-cellulose, poly-adenosine, poly-thymine, and combinations thereof.

In some embodiments, following complexing at a specified pH as discussed above, e.g., at a pH greater than 8 or less than 3, it may be beneficial to remove supernatant from the mixture formed by contacting the beneficial agent with the complexing agent so at to remove non-complexed, e.g., non-charge-neutralized, beneficial agent, prior to use of the beneficial agent complex in the compositions disclosed herein.

In some embodiments, a cationic agent is complexed with the beneficial agent to form the insoluble beneficial agent complex. Suitable cationic agents may include, but are not limited to, protamine, poly-lysine, poly-arginine, polymyxin, Ca²⁺ and Mg²⁺. Anionic agents may also be utilized as appropriate to form the insoluble beneficial agent complex. Suitable anionic agents may include, but are not limited to, CMC as mentioned above as well as poly-adenosine and poly-thymine. Where the anionic agent is poly-adenosine, the poly-adenosine may be, for example, a 10mer to a 150mer. Where the anionic agent is poly-thymine, the poly-thymine may be, for example, a 10mer to a 1500mer.

Two or more precipitating agents and/or stabilizing agents may be utilized in combination to facilitate formation of the insoluble beneficial agent complexes described herein, e.g., for improved chemical or physical stability of the beneficial agent in the complex and/or improved drug release kinetics, e.g., reduced burst effect and/or a sustained delivery profile. For example, the combination of protamine and a divalent metal or salt thereof with a protein beneficial agent may form an insoluble complex which when dispersed in the vehicle of the disclosed compositions provides a composition having a desired beneficial agent release profile in vivo. In addition, such combinations of precipitating and/or stabilizing agents may improve the chemical and physical stability of the beneficial agent complex and render the complex more resistant to sterilization conditions, e.g., radiation sterilization, including electron beam sterilization and gamma radiation sterilization.

Accordingly, in some embodiments the insoluble beneficial agent complex includes beneficial agent in combination with both protamine and a divalent metal or salt thereof (e.g. Zn²⁺ or Zinc acetate). The molar ratio of beneficial agent:divalent metal or salt:protamine (e.g., beneficial agent:zinc:protamine) may be in the range of 1:0.5 to 2.0:0.3 to 0.5.

Protamine may be used alone or in combination with one of the precipitating agents and/or stabilizing agents described above to form an insoluble beneficial agent complex according to the present disclosure. In some embodiments, e.g., where the composition is to be administered to a human or non-human animal, it may be desirable to include an additive such as methionine in order to provide a radiation-stable composition. This may be useful for example, where the beneficial agent is a protein or a peptide. Methionine may be added, e.g., to the composition prior to lyophilization or spray-drying to form of an insoluble beneficial agent complex powder which can be sterilized, e.g., via gamma irradiation, either before or after combining the powder with a vehicle as described herein.

In some embodiments, the composition maintains a purity of at least 90% or greater (e.g., 95%) for a period of at least 24 hours following exposure to gamma irradiation at a dose of 25 kGy. In some embodiments, a purity of at least 90% or greater (e.g., 95%) is maintained for a period of at least one month.

The insoluble beneficial agent complexes are present in the composition in the form of insoluble particles. The size of these particles may differ depending on the methods used to prepare the beneficial agent complex. Typically, the particles are small enough to pass through a small needle, such as a 25 gauge needle. In some embodiments the insoluble beneficial agent complex is dispersed in the vehicle in the form of particles having an average size ranging from about 1 μm to about 400 μm in diameter or in largest dimension, e.g., from about 1 μm to about 300 μm, from about 1 μm to about 200 μm, from about 1 μm to about 100 μm, from about 1 μm to about 90 μm, from about 1 μm to about 80 μm, from about 1 μm to about 70 μm, from about 1 μm to about 60 μm, from about 1 μm to about 50 μm, from about 1 μm to about 40 μm, from about 1 μm to about 30 μm, from about 1 μm to about 20 μm, or from about 1 μm to about 10 μm in diameter or in largest dimension.

In some embodiments, the insoluble beneficial agent complex is dispersed in the vehicle in the form of particles having an average size ranging from about 10 μm to about 100 μm in diameter or in largest dimension. Particles sizes in this range in combination with density matching, e.g., wherein the density of the particles is the same or similar to the density of the vehicle, contribute to the improved syringeability and injectability of the compositions disclosed herein.

In some embodiments, the density of the insoluble particles is approximately the same as the density of the vehicle in which the particles are dispersed. This provides for increased physical stability of the particles in the vehicle and improved dispersion of the particles in the vehicle particularly during storage of the compositions, e.g., at low temperatures such as 2-8° C. For example, in some embodiments, both the particles and the vehicle have a density of between about 0.9 and 1.2 g/cm³. In some embodiments, the average density of the particles does not differ from that of the vehicle by more than 0.25 g/cm³, e.g., by more than 0.20 g/cm³, by more than 0.15 g/cm³, or by more than 0.05 g/cm³. In some cases, the apparent density of the vehicle is within 10%, e.g., within 8%, within 5%, or within 3%, of the apparent density of the particles.

### Additional Components

A variety of additional components may be added to the disclosed compositions provided they do not substantially disrupt the beneficial characteristics of the compositions as discussed herein, e.g., viscosity, etc. Suitable components may include, but are not limited to, one or more pharmaceutically acceptable excipients, e.g., stabilizers, dyes, fillers, preservatives, buffering agents, antioxidants, wetting agents, anti-foaming agents and the like. Additional components may include, e.g., sucrose, polysorbate, methionine, etc.

For example, methionine may be included in a composition of the present disclosure as an antioxidant, and in some embodiments sucrose is included as a stabilizer. As discussed above, methionine may be combined with an insoluble beneficial agent complex as described herein to form a radiation stable powder or a radiation stable composition as described herein.

In some embodiments, a high-viscosity carrier such as sucrose acetate isobutyrate (SAIB) may be included in a composition of the present disclosure. For example, SAIB may be included in an amount ranging from about 5% to about 20%, such as about 5% to about 10%, by weight of the vehicle.

In some embodiments, the vehicle comprises about 5% to 10% SAIB, about 70% to about 75% of the hydrophobic solvent, and about 15% to 25% of the biodegradable polymer, wherein each % is % by weight of the vehicle. In one or more embodiments, the vehicle comprises about 5 to about 10% SAIB, about 65% to about 70% benzyl benzoate, about 3% to about 7% ethanol, and about 15% to about 25% poly(lactic-co-glycolic acid) (PLGA), wherein each % is % by weight of the vehicle. In some embodiments, the vehicle comprises about 15% to about 25% SAIB, about 55% to about 65% benzyl benzoate, about 5% to about 15% benzyl alcohol, and about 5% to about 15% polylactic acid (PLA), wherein each % is % by weight of the vehicle. In one or more embodiments, the vehicle comprises about 65% to about 75% benzyl benzoate, about 5% to about 15% benzyl alcohol, and about 15% to about 25% polylactic acid (PLA), wherein each % is % by weight of the vehicle.

In one or more embodiments, inclusion of SAIB at 8% by weight of the vehicle, allows for inclusion of the hydrophobic solvent at 72%, by weight of the vehicle and inclusion of the biocompatible, biodegradable polymer at 20% by weight of the vehicle. In some embodiments, the amount of SAIB in the composition may be adjusted provided that the weight % of the hydrophobic solvent is maintained between about 60 and about 95% by weight of the vehicle and the weight % of the biocompatible, biodegradable polymer is maintained between about 5 and about 40% by weight of the vehicle.

For instance, the amount of SAIB may be adjusted from 0 to 35% by weight of the vehicle, e.g., in 1% intervals, provided that the percentages of the hydrophobic solvent and the biocompatible, biodegradable polymer are adjusted accordingly, preferably provided that the zero shear viscosity of the resulting composition does not exceed 1,200 cP at 25° C. Without reciting each combination of the above three components that fall within the specified ranges, it is to be understood that all such combinations are within the scope of the present disclosure and further that this is intended to provide antecedent basis for specific recitations of any combination of the above three components that meet the above range and viscosity recitations.

### Methods of Preparation

In general, the present compositions may be made by any of the various methods and techniques known and available to those skilled in the art.

The compositions of the present disclosure may be prepared generally by combining a biodegradable polymer as described herein and a hydrophobic solvent as described herein to form a vehicle of the composition. The biodegradable polymer is typically provided in an amount of from about 5% to about 40% by weight of the vehicle, and the hydrophobic solvent is typically provided in an amount of from about 95% to about 60% by weight of the vehicle. The insoluble component comprising beneficial agent, e.g., an insoluble beneficial agent complex, is dispersed in the vehicle. Such dispersion may occur following one or more milling or sieving steps to obtain particles of a desired size. One or more homogenization steps may be utilized following dispersion of the insoluble beneficial agent or insoluble beneficial agent complex in the vehicle. It should be noted that within the above ranges the % by weight of the biodegradable polymer and the hydrophobic solvent may be adjusted while maintaining a desired viscosity range, e.g., a zero shear viscosity less than 1,200 centipoise (cP), e.g., less than 1000 cP, less than 500 cP or less than 100 cP at 25° C. In addition, one or more additional components may be included in the vehicle as described previously herein.

Insoluble beneficial agent complex particles may be prepared, for example, by dissolving the beneficial agent in a suitable buffer and subsequently adding a suitable amount of a stabilizing/precipitating agent until a precipitate is formed at a temperature greater than the freezing point but less than the boiling point of the buffer. The suitable buffer with dispersed precipitate is then subjected to a suitable drying process, e.g., spray drying or lyophilization, to provide a powder comprising insoluble beneficial agent complex. Alternatively, the precipitate can be recovered by centrifugation and removal of the resulting supernatant. It can then be re-suspended in aqueous medium for spray drying or lyophilized directly. One or more size reduction and sieving steps may be utilized to adjust the particle size of the beneficial agent complex. The complexed powder is mixed with a suitable amount of the prepared vehicle to disperse the beneficial agent complex particles in the vehicle. In some embodiments, where the beneficial agent is a low molecular weight compound, the beneficial agent complex may include only the salt form of the beneficial agent, provided that the salt form of the beneficial agent is at least substantially insoluble in the vehicle. The formulation may be sterilized prior to use using any suitable method known in the art, e.g., gamma sterilization at a dose of 10 kGy or greater. Alternatively, the beneficial agent complex and the vehicle may be sterilized separately and then combined prior to use.

### Biodegradable Formulations

As discussed previously herein, in some embodiments, the biodegradable compositions of the present disclosure include A) a single phase vehicle including i) a biodegradable polymer present in an amount of from about 5% to about 40% (e.g., from about 6% to about 29%, from about 7% to about 28%, from about 8% to about 27%, from about 9% to about 26%, from about 10% to about 25%, from about 11% to about 24%, from about 12% to about 23%, from about 13% to about 22%, from about 14% to about 21%, from about 15% to about 20%, from about 16% to about 19%, or from about 17% to about 18%) by weight of the vehicle, and ii) a hydrophobic solvent present in an amount of from about 95% to about 60% (e.g., from about 94% to about 61%, from about 93% to about 62%, from about 92% to about 63%, from about 91% to about 64%, from about 90% to about 65%, from about 89% to about 66%, from about 88% to about 67%, from about 87% to about 68%, from about 86% to about 69%, from about 85% to about 70%, from about 84% to about 71%, from about 83% to about 72%, from about 82% to about 73%, from about 81% to about 74%, from about 80% to about 75%, from about 79% to about 76%, or from about 78% to about 77%) by weight of the vehicle; and B) an insoluble component comprising beneficial agent, e.g., an insoluble beneficial agent complex, dispersed in the vehicle, wherein the biodegradable composition has a zero shear viscosity less than 1,200 centipoise (cP) (e.g., less than 1100 cP, less than 1000 cP, less than 900 cP, less than 800 cP, less than 700 cP, less than 600 cP, less than 500 cP, less than 400 cP, less than 300 cP, less than 200 cP, or less than 100 cP) at 25° C., is injectable through a small gauge needle and is not an emulsion or gel.

In some embodiments, a biodegradable composition of the present disclosure has a zero shear viscosity less than 1,200 cP (e.g., less than 1100 cP, less than 1000 cP, less than 900 cP, less than 800 cP, less than 700 cP, less than 600 cP, less than 500 cP, less than 400 cP, less than 300 cP, less than 200 cP, or less than 100 cP) at 25° C.

It should be noted that the amount of the biodegradable polymer and the amount of the hydrophobic solvent may be varied, for example, to achieve a desired viscosity, e.g., in 1% by weight increments, provided that they are typically maintained within about 5% to about 40% by weight of the vehicle and about 95% to about 60% by weight of the vehicle, respectively. Accordingly, without reciting every possible combination falling within the above ranges, this is intended to provide antecedent basis for such combinations.

In some embodiments, the zero shear viscosity of the biodegradable composition is from about 1000 cP to about 100 cP, e.g., about 900 cP to about 100 cP, about 800 cP to about 100 cP, about 700 cP to about 100 cP, about 600 cP to about 100 cP, about 500 cP to about 100 cP, about 400 cP to about 100 cP, about 300 cP to about 100 cP, or about 200 cP to about 100 cP at 25° C.

In some embodiments, in addition to a relatively low viscosity at 25° C., the disclosed biodegradable compositions also exhibit relatively low viscosity at 37° C., e.g., a zero shear viscosity less than 500 cP, less than 400 cP, less than 300 cP, less than 200 cP, or less than 100 cP. In some embodiments, the zero shear viscosity of the biodegradable composition is from about 500 cP to about 100 cP, from about 400 cP to about 200 cP, or about 300 cP at 37° C. The viscosity of these formulations declines with increasing temperature; frequently in exponential fashion.

The disclosed biodegradable compositions also typically exhibit relatively low viscosity (e.g., a zero shear viscosity less than 500 cP, less than 400 cP, less than 300 cP, less than 200 cP, or less than 100 cP) at 37° C. after being exposed to phosphate-buffered saline in vitro, and maintain this low viscosity over time, e.g., for at least 5 hrs, at least 24 hrs, at least 48 hrs, at least 72 hrs, or at least 168 hrs, of exposure to phosphate-buffered saline.

Surprisingly, the disclosed biodegradable depot compositions typically demonstrate good syringeability and injectability while providing for sustained release of the beneficial agent in-vivo with minimal burst. Syringeability and injectability may be characterized by the time it takes to inject a known volume of the biodegradable depot composition through a syringe of known size fitted with a relatively small gauge needle, e.g., a 1-5 mL syringe fitted with a needle having a gauge of about 21 to about 27. In some embodiments, the biodegradable depot compositions of the present disclosure may be characterized as having good syringeability and injectability based on their ability to be injected through a 1 ml syringe fitted with an approximately 0.5 in needle having a gauge of about 21 to about 27, wherein a 0.5 ml volume of the biodegradable depot can be injected in less than 25 sec (e.g., less than 20 sec., less than 15 sec, less than 10 sec, or less than 5 sec) at 25° C. with the application of a 5 to 10 lb force. In some embodiments, under the above conditions, the biodegradable depot can be injected in a range of from about 25 sec to about 1.5 sec, e.g., from about 20 sec to about 1.5 sec, from about 15 sec to about 1.5 sec, from about 10 sec to about 1.5 sec, or from about 5 sec to about 1.5 sec.

In addition to good injectability and syringeability as described herein, in some embodiments, the biodegradable compositions of the present disclosure demonstrate minimal burst and sustained delivery of beneficial agent over time. “Minimal burst” may be characterized in terms of Cₘₐₓ/Cₘᵢₙ, wherein the acceptable Cₘₐₓ/Cₘᵢₙ upper limit may vary depending on the beneficial agent to be delivered. In some embodiments, the weight % of beneficial agent released as burst over the first 24 hours is less than 30% of the total amount released over one week, e.g., less than 20% or less than 10%, of the total amount released over one week. In some embodiments, the weight % of beneficial agent released as burst over the first 24 hours is less than 10% of the total amount released over one month, e.g., less than 8% or less than 5%, of the total amount released over one month. As used herein, “sustained delivery” refers to durations which are at least several fold, e.g., at least 5 fold to at least 10 fold, longer than the duration obtained from a single dose of an immediate-release (IR) formulation of the same beneficial agent (determined by Adsorption, Distribution, Metabolism, and Excretion (ADME) characteristics of the beneficial agent itself).

As mentioned above, the disclosed biodegradable compositions provide for sustained release of the beneficial agent in-vivo with minimal burst effect in addition to possessing good injectability, syringeability and chemical stability as discussed above. This is an unexpected and surprising result as currently available formulations generally provide either controlled release or injectability/syringeability but not both. For example, commercially available depot formulations may rely on the formation of an extremely viscous polymer matrix to provide controlled release of a beneficial agent. However, such formulations have poor injectability/syringeability due to the viscous nature of the depot. Alternatively, other commercially available formulations utilize vehicles which may have good injectability/syringeability due to a high-solvent content but poor control over release of the beneficial agent. Moreover, one would expect a low viscosity liquid composition such as those disclosed herein to have poor release kinetics in the form of a substantial burst effect and an exponentially declining delivery profile. Contrary to this expectation, the present compositions demonstrate low burst effect and good control over release of the beneficial agent over a period of one day to one month or longer.

Without intending to be bound by any particular theory, it is believed that the beneficial release characteristics of the compositions of the present disclosure are due at least in part to the formation of a fluid, non-structured (without any appreciable mechanical integrity), “rate-controlling cloud” or “rate-controlling film” at the surface of the composition in vivo. The rate-controlling cloud or film can be characterized as occurring at the surface of the composition in the aqueous environment. The desirable controlled delivery characteristic of the disclosed compositions may result from the rate-controlling contributions of both the insoluble component comprising beneficial agent, e.g., an insoluble beneficial agent complex, dispersed in the liquid core of the composition and the polymer cloud or film on the surface of the composition. In addition, in some embodiments, a synergistic effect with respect to release rate control, e.g., as demonstrated by MRT, is seen as an apparent result of interaction between the beneficial agent complex and the rate controlling cloud or film. While the rate controlling cloud or film lacks appreciable mechanical integrity, it has a measureable thickness less than 10 μm.

In some embodiments, the compositions of the present disclosure lack of gel forming or gelling characteristics. For example, many prior art vehicle compositions exhibit gel formation when aged at 37° C. which can be characterized by an increase in the storage modulus relative to the loss modulus. In contrast, the compositions of the present disclosure can be characterized by a relatively large G″/G′ ratio, e.g., a G″/G′ ratio of greater than or equal to 10, such as greater than or equal to 15 or greater than or equal to 20, following aging at 37° C. for a period of 14 days, wherein G″ is the loss modulus and G′ is the storage modulus.

In certain embodiments, the compositions are Newtonian. For instance, in some cases, the viscosity of the composition at 25° C. varies less than 7%, less than 6%, less than 5%, less than 4%, or less than 3%, when measured at a shear rate ranging from 7 sec⁻¹ to 500 sec⁻¹.

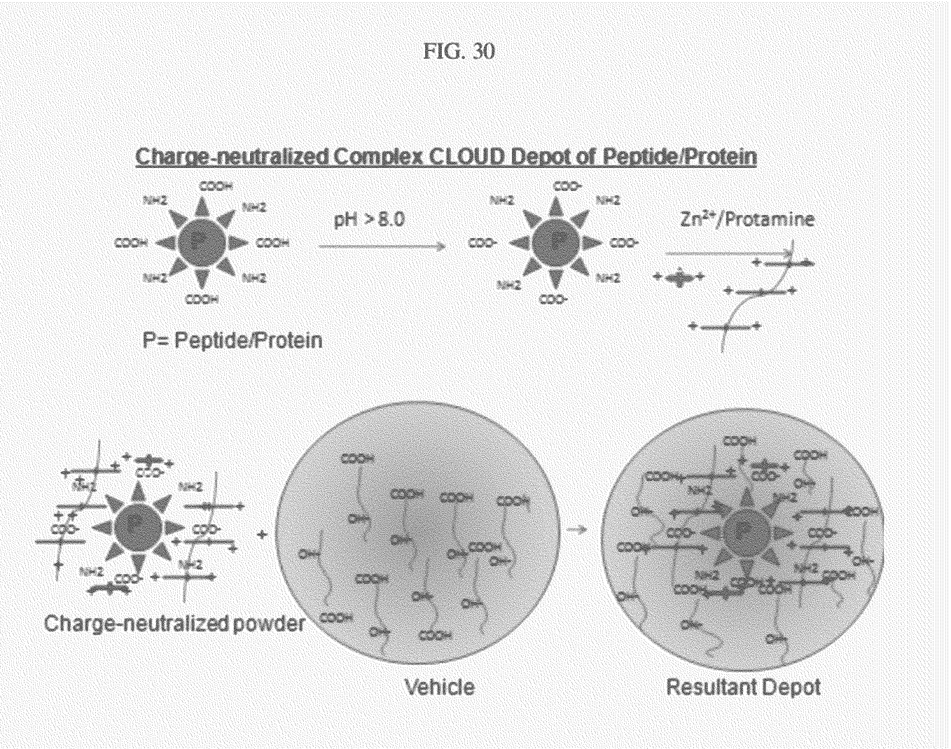

Without intending to be bound by any particular theory, FIG. 30 is provided as a representation of a composition comprising a charge-neutralized complex of a beneficial agent containing acid groups such as a peptide or protein. During the event of charge neutralization, either peptide or protein or any acid terminated molecule can become negatively charged at basic pH (pH>8) in the presence of buffer. The charged beneficial molecule in aqueous solution will be neutralized with solution of positively charged counter-ion such as protamine or Zn²⁺ ion at an optimal molar ratio. This molar concentration of either protamine or zinc ion is obtained by titration of protamine or zinc ion against the fixed concentration of negatively charged peptide or protein. The molar concentration of either protamine or zinc ion will also depend on the net charge on the protein or peptide and its molar concentration. The aqueous solubility of charge-neutralized complex (peptide or protein plus counter-ion) is dramatically reduced and it will precipitate out of solution. Any charged species of protein or peptide and counter-ion remain in the solution. The dried powder of insoluble beneficial agent—counter-ion complex can be uniformly dispersed in a polymer solution (vehicle) either by hand or mechanical mixing (e.g. homogenization). The resultant formulation controls the release of the beneficial agent via solubility, dissolution rate, and diffusivity. Electrostatic, hydrogen bonding and hydrophobic interactions may also occur between the dispersed particles of charge-neutralized beneficial agent and polymer, and these may also modulate the release kinetics as manifested by the surprising contribution by the polymer-complex interaction to MRT of the beneficial agent in vivo.

In some embodiments, the disclosed compositions are suspensions that remain substantially homogenous for about 3 months, even more preferably for about 6 months, and yet even more preferably, for about 1 year. In one or more embodiments, the insoluble beneficial agent complex remains physically and chemically stable in the suspension vehicle for about 3 months, even more preferably for about 6 months, and yet even more preferably, for about 1 year.

### Administration of Biodegradable Formulations

As discussed previously herein, the disclosed biodegradable formulations possess low viscosity along with good injectability and syringeability making them well suited for delivery via a syringe (e.g., a 1-5 mL syringe) with a narrow gauge needle, e.g., 21 to 27 gauge. In addition, the injectable depot formulations may also be delivered via one or more needless injectors known in the art.

Suitable routes of administration include, but are not limited to, subcutaneous injection and intramuscular injection. Suitable routes of administration also include, for example, intra-articular and intra-ocular, e.g., intra-vitreal, administration for local delivery.

The formulations disclosed herein may also find use in oral formulations, e.g., formulations delivered in a gel-cap (soft or hard) or as a mouthwash.

The formulations disclosed herein may also find use as coatings for medical devices, e.g., implantable medical devices. Such coatings may be applied, e.g., by dip-coating the medical device prior to implantation.

The formulations of the present disclosure may be formulated such that a desired pharmacological effect is achieved via administration on a periodic basis. For example, the formulations may be formulated for administration on a daily, weekly or monthly basis.

The actual dose of the beneficial agent or insoluble beneficial agent complex to be administered will vary depending on the beneficial agent, the condition being treated, as well as the age, weight, and general condition of the subject as well as the severity of the condition being treated, and the judgment of the health care professional. Therapeutically effective amounts are known to those skilled in the art and/or are described in the pertinent reference texts and literature.

For example, in the case of proteins and peptides beneficial agents, the beneficial agent will typically be delivered such that plasma levels of the beneficial agent are within a range of about 5 picomoles/liter to about 200 picomoles/liter. On a weight basis, a therapeutically effective dosage amount of protein or peptide will typically range from about 0.01 mg per day to about 1000 mg per day for an adult. For example, peptide or protein dosages may range from about 0.1 mg per day to about 100 mg per day, or from about 1.0 mg per day to about 10 mg/day.

In some embodiments, a suitable low molecular weight compound may be characterized as one which can provide the desired therapeutic effect with a dose of less than or equal to about 30 mg/day as delivered from a depot administered once a week, or a dose of less than or equal to about 10 mg/day as delivered from a depot administered once a month. For example, a suitable low molecular weight compound may be one which can provide the desired therapeutic effect with a dose of less than about 30 mg/day, e.g., less than about 25 mg/day, less than about 20 mg/day, less than about 15 mg/day, less than about 10 mg/day, less than about 5 mg/day or less than about 1 mg/day as delivered from a depot administered once a week. In some embodiments, a suitable low molecular weight compound is one which can provide the desired therapeutic effect with a dose of from about 30 mg/day to about 1 mg/day, e.g., from about 25 mg/day to about 5 mg/day, or from about 20 mg/day to about 10 mg/day as delivered from a depot administered once a week.

Similarly, a suitable low molecular weight compound may be one which can provide the desired therapeutic effect with a dose of less than about 10 mg/day, less than about 9 mg/day, less than about 8 mg/day, less than about 7 mg/day, less than about 6 mg/day, less than about 5 mg/day, less than about 4 mg/day, less than about 3 mg/day, less than about 2 mg/day or less than about 1 mg/day as delivered from a depot administered once a month. In some embodiments, a suitable low molecular weight compound may be one which can provide the desired therapeutic effect with a dose of from about 10 mg/day to about 1 mg/day, e.g., from about 9 mg/day to about 2 mg/day, from about 8 mg/day to about 3 mg/day, from about 7 mg/day to about 4 mg/day, or from about 6 mg/day to about 5 mg/day as delivered from a depot administered once a month.

In some embodiments, e.g., where the formulation may have been in storage for a period of time prior to injection, the formulation may be mixed, e.g., via shaking, prior to administration to ensure that the insoluble component comprising beneficial agent, e.g., an insoluble beneficial agent complex, is sufficiently dispersed in the vehicle carrier.

### Kits

A variety of kits may be provided which include one or more components of the biodegradable formulations disclosed herein along with instructions for preparing and/or using the same. For example, in one embodiment, a suitable kit may include a vehicle as described herein in a first container and an insoluble component comprising beneficial agent, e.g., an insoluble beneficial agent complex, as described herein in a second container, e.g., in powder form. These components may then be mixed together prior to injection to form a biodegradable formulation according to the present disclosure. In some embodiments, the first container is a syringe which may be coupled to the second container, e.g., a vial with a luer lock, to provide a mechanism for mixing the vehicle and the insoluble component comprising beneficial agent, e.g., an insoluble beneficial agent complex. In other embodiments, both the first and second containers are syringes which may be coupled, e.g., via a luer lock, to provide a mechanism for mixing the vehicle and the insoluble component comprising beneficial agent, e.g., an insoluble beneficial agent complex.

In another embodiment, the biodegradable formulation may be provided pre-mixed in a single container, e.g., a single syringe.

In another embodiment, the biodegradable formulation may be provided un-mixed in a pre-filled, dual-chamber syringe including a first chamber containing the vehicle and a second chamber containing the insoluble component comprising beneficial agent, e.g., an insoluble beneficial agent complex. The syringe may be provided such that a user can initiate contact and subsequent mixing of the vehicle and the insoluble component comprising beneficial agent, e.g., an insoluble beneficial agent complex.

The instructions for use of the kit and/or kit components may be provided as complete written instructions along with the kit, e.g., as an insert card or printed on the kit packaging; or stored on a computer readable memory device provided with the kit. Alternatively, the kit may include instructions which provide a brief instruction to the user and direct the user to an alternate source for more complete use instructions. For example, the kit may include a reference to an internet site where the complete instructions for use may be accessed and/or downloaded.

### EXAMPLES

The following examples are put forth so as to provide those of ordinary skill in the art with a disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight as measured by gel permeation chromatography, temperature is in degrees Celsius, and pressure is at or near atmospheric. Standard abbreviations may be used, e.g., bp, base pair(s); kb, kilobase(s); kd, kiloDalton(s); pL, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1

### Preparation of rhGH-Protamine Complex

Spray Drying

A spray dried powder formulation of hGH (BresaGen) complexed with protamine sulfate was prepared as follows. 1.00 g of BresaGen rhGH powder was placed in a 150 mL wide-mouth glass jar. 55 mL of a 25 mM NH₄HCO₃ (pH˜7.5) solution was added and the compound was stirred for 30 min at room temperature, 400 rpm until it became clear. 1.9 mL of a 290 mM sucrose solution was then added while stifling at 400 rpm. When the solution was clear 152 μL of a 10% polysorbate 20 solution was added. 12.9 mL of protamine sulfate solution (conc. 10 mg/mL) was then added slowly to form a white precipitate. The mixture was stirred for 30 min before spray drying to complete the complexation reaction.

For formulations including a divalent metal or salt thereof (e.g., zinc acetate) in addition to protamine, such components may be added to the desired ratio prior to the addition of protamine. For example, a 100 mM stock solution of zinc acetate may be utilized to add zinc acetate to the desired ratio.

The spray dry conditions were as follows:
Inlet temperature set up: 140° C.,
Aspirator 100%,
Pump: 13%,
Nozzle Cleaner: 2 pulses per min.

Following spray drying, the yield of the complexed powder was 1.1066 g. The rhGH content in the complexed powder was determined via HPLC as follows. The powder was dissolved in 2% phosphoric acid and the clear solution was run on an HPLC system. The rhGH content of the powder was found to be 75% by weight. The complexed powder was subsequently transferred to 3 mL glass syringes, sealed and stored in foil pouches under refrigeration.

Lyophilization

As an alternative to the spray drying process described above, the insoluble beneficial agent complex of the present disclosure may be provided using a lyophilization process. An exemplary lyophilization process is described below.

1.00 g of BresaGen hGH powder was placed in a 150 mL wide-mouth glass jar. 55 mL of 25 mM NH₄HCO₃ (pH˜7.5) solution was added and the compound was stirred for 30 min at room temperature, 400 rpm until it became clear. 1.9 mL of 290 mM sucrose solution was then added while stirring at 400 rpm. When the solution was clear, 1541, of 10% polysorbate 20 solution was added. Then, 12.9 mL of protamine sulfate solution (conc. 10 mg/mL) was slowly added to form a white precipitate. The resulting suspension was stirred for 30 min to complete the complexation reaction.

Aliquots of 3 mL each of the bulk suspension from the above step were transferred into 5 mL type-Hypak BD glass syringes and lyophilized using the lyophilization cycle provided in Table 1 and a program P90 (optimized for hGH) to fit the steps provided with an FTS lyophilizer, Dura Stop, MP Stoppering Tray Dryer, Stone Ridge, N.Y. The final amount of beneficial agent in each syringe was 50 mg. The syringes were sealed and pouched and stored in a −20° C. freezer for further study.

TABLE 1

| | SHELF | | Chamber | |
|---|---|---|---|---|
| | TEMP | TIME | pressure | |
| STEP | (° C.) | (HOUR) | (mTorr) | Modifications |
| FREEZING | PRECOOL @ −40 | | Not | 1 hr prior loading |
| | | | controlled | Instrument was pre- |
| | | | | cooled |
| | −40 | 2.0 | 3000 | Modified to fit the |
| | | | | freezing steps |
| | | | | available for FTS |
| | | | | Lyophilizer |
| PRIMARY | −25 | 2.0 | 100 | |
| DRYING | −30 | 35.0 | | |
| SECONDARY | 25 | 2.0 | | Actual time was 24.7 |
| DRYING | | | | hours due to the slight |
| | | | | variation of ramping |
| | | | | speed (setting is |
| | | | | 2 digits after decimal |
| | | | | point) |
| | 25 | 10.0 | | |
| | 5 | 10.0 | 200 | Actual hold time was 8 |
| | | | | hours |

### Example 2

### Preparation and In-Vivo Evaluation of rhGH Biodegradable Drug Delivery Depot Formulation

Preparation

Five different formulations of rhGH-Protamine complex and vehicle were prepared and tested. The formulations were prepared as indicated below using the following materials: Benzyl benzoate, Spectrum; SAIB, Pharmaceutical grade, DURECT; and PLA, Poly (DL-Lactide), MW 15100 Da, DURECT Corporation. The five formulations included:
1) rhGH-Protamine (1:0.5 molar ratio) suspended in phosphate buffered saline (PBS),
2) rhGH-Protamine suspended in Benzyl benzoate (BB),
3) rhGH-Protamine suspended in SAIB/BB 8/92% w/w (stock vehicle prepared by mixing 4.002 g of SAIB with 46.017 g of Benzyl benzoate in a 100 mL glass jar and sonicating at RT for 30 minutes),
4) rhGH-Protamine suspended in BB/PLA (DURECT) 80/20% w/w (stock vehicle prepared by mixing 20.015 g of Benzyl benzoate with 5.007 g of PLA in a 100 mL glass jar and sonicating at RT for 30 minutes), and
5) rhGH-Protamine suspended in SAIB/BB/PLA (DURECT) 8/72/20% w/w (stock vehicle prepared by weighing 20.014 g of PLA in a 100 mL glass jar and mixing with 72.309 g of Benzyl benzoate and 8.147 g of SAIB, followed by sonication for 30 minutes at RT).

Injectable formulations having components as indicated in 1-5 above were prepared as follows:

A) The foil pouches with the syringes containing the complexed powder were removed from refrigeration and placed in a clean, dry area at room temperature for a minimum of 60 minutes prior to opening;

B) Vials containing the vehicle were also placed in a clean, dry area at room temperature for at least 60 minutes prior to opening;

C) After the foil pouches were allowed to equilibrate, each pouch was opened with clean scissors and the syringes were removed while being careful not to cut any of the pouch contents;

D) For each test article, 1 mL of vehicle was withdrawn from a stock solution with a 1 mL syringe (Excel or equivalent) fitted with a 16Ga, 1 inch needle (BD PN305197 or equivalent);

E) The plastic tip was removed from the 3 mL glass syringe containing the test article powder;

F) One side of a sterile female-female luer adaptor was fixed to the syringe of Step E;

G) The 1 mL syringe containing vehicle (Step D) was connected to the other side of the sterile female-female luer;

H) The entire liquid contents of the 1 mL syringe were pushed through the female-female luer into the powder contents of the 3 mL glass syringe;

I) The syringes were left connected for at least 10-15 minutes to allow the vehicle to wet the powder;

J) The vehicle was mixed with the powder by passing the mixture between the two syringes until a uniform suspension was produced (at least 20 passes between the syringes);

K) The required volume contents were pushed (animal dose+50 uL for dead space) into the 1 mL Excel syringe and the 3 mL glass syringe was uncoupled;

L) The female-female luer was then removed from 1 mLExcel syringe;

M) A 21Ga, 1 inch needle (Terumo, UTW or equivalent) was then placed into the luer lock of the 1 mL syringe with volume markings and the needle was primed with test article suspension. The syringe was then ready for dosing animal 1.

N) To prepare additional animal dosages, the female-female luer was attached to the 3 mL glass syringe and a new 1 mL Excel syringe was attached. The required volume (2^{nd} animal dose+50 uL for dead space) was then pushed into a 1 mL Excel syringe and the 3 mL glass syringe was uncoupled. The female-female luer was then removed from the 1 mL Excel syringe;

O) A 21Ga, 1 inch needle (Terumo, UTW or equivalent) was then placed into the luer lock of the 1 mL syringe with volume markings and the needle was primed with test article suspension. The syringe was then ready for dosing animal 2. This process was continued as needed until all animals were dosed.

In-Vivo Administration and Monitoring

In-vivo experiments were conducted as follows. Sprague-Dawley rats were dosed via subcutaneous bolus injection and monitored for a one week period. Six experimental treatment groups were utilized with six animals per group. These groups utilized the five formulations described above and a reference formulation, rhGH in PBS without protamine (Aq. Soln.). For both the rhGH in PBS and the rhGH-protamine formulation (Aq. complex), delivery was via 300 μl injection of a 10 mg/ml formulation to achieve a 3 mg dose. For each of rhGH-protamine in benzyl benzoate (BB) 100% w/w, rhGH-Protamine in SAIB/BB (8/92) % w/w, rhGH-Protamine in BB/PLA (80/20) % w/w, and rhGH-Protamine in SAIB/BB/PLA (8/72/20) % w/w, delivery was via 100 μl injection of a 50 mg/ml formulation to achieve a 5 mg dose.

Blood samples were taken at 0.5, 1, 2, 4, 8, 12, 24 and 48 hrs following dosing for the rhGH (PBS) formulation while samples for each of the rhGH-protamine formulations were taken at 1, 4, 8, 12, 24, 48, 72, 120 and 168 hrs. Serum rhGH profiles were determined via ELISA.

Results

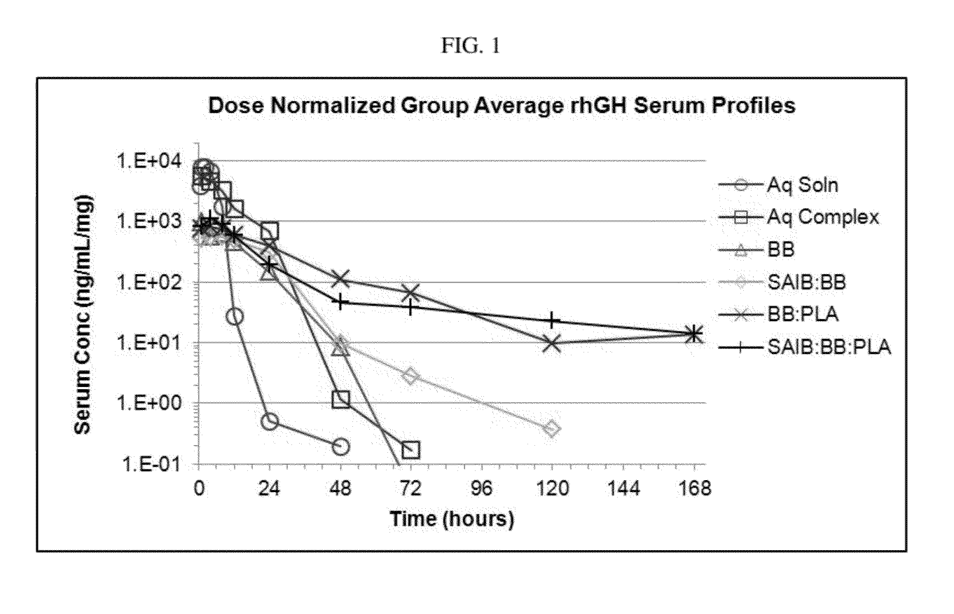

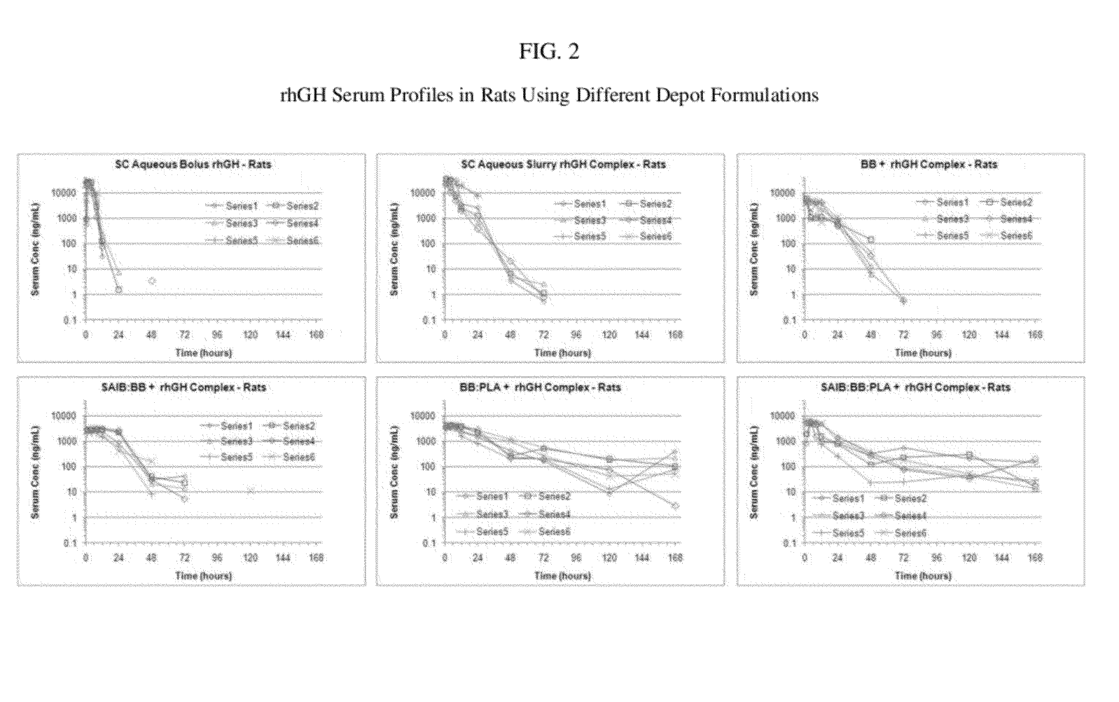

FIG. 1 shows the dose-normalized, group-average serum rhGH profiles, for the reference and the five test formulations following subcutaneous dosing. FIG. 2 plots serum rhGH concentrations over time for each animal in each test group. These plots allow one to discern readily the effects of complexation and the vehicles, and also show the inter-animal variability. (Note: all non-zero concentrations were plotted).

Relative to the aqueous solution (rhGH in PBS without protamine), serum levels from the complex suspended in PBS were maintained for an additional 24 hrs. Suspending the rhGH complex in BB reduced 0-24 hr plasma levels 6-8 fold, but did not prolong protein delivery relative to the complex suspended in PBS. Addition of SAIB to BB extended delivery by about 48 h. Adding 20% (w/w) acid-initiated PLA (M_{w}˜14.5 kDa) to BB extended rhGH delivery to beyond 168 hrs., but substitution of 8% w/w SAIB for BB in BB:PLA 80:20% w/w had no significant effect on rhGH delivery.

These results indicate that, in-vivo, the protamine complex reduced initial serum levels and prolonged delivery relative to a s.c. aqueous bolus of rhGH in solution. Dispersing the complex in BB reduced initial release relative to the protamine complex without vehicle, but did not extend the overall duration of delivery. Addition of 8% SAIB to BB provided a modest extension of release relative to BB alone, but addition of 20% PLA to BB greatly extended delivery of protein. Lastly, addition of 8% SAIB to BB:PLA provided no further extension of delivery.

### Example 3

### In-Vivo Evaluation in Rat for IFNα2a Biodegradable Drug Delivery Depot Formulation

The following formulations were administered subcutaneously to rats, and IFNα2a serum concentration was monitored over time:
A) 2.5 mg/ml IFNα2a formulation with 1% sucrose and protamine-zinc (spray

dried), dispersed in a SAIB/BB/PLA (8:72:20, % w/w) vehicle; and
B) 2.5 mg/ml IFNα2a formulation with 1% sucrose and protamine-zinc (spray dried), dispersed in a SAIB/BB/PLGA (8:72:20, % w/w) vehicle.

For each formulation the ratio of IFNα2a to Zn²⁺ to protamine in the complex was (1:1:0.3 m/m). The protein dose was 0.5 mg for each formulation. Methionine was added to each formulation to prevent oxidation of protein. Rats were immune suppressed with cyclosporine and methyl-prednisolone. Injections were via Excel 1 ml syringes using 23 gauge ⅝ inch Terumo needles.

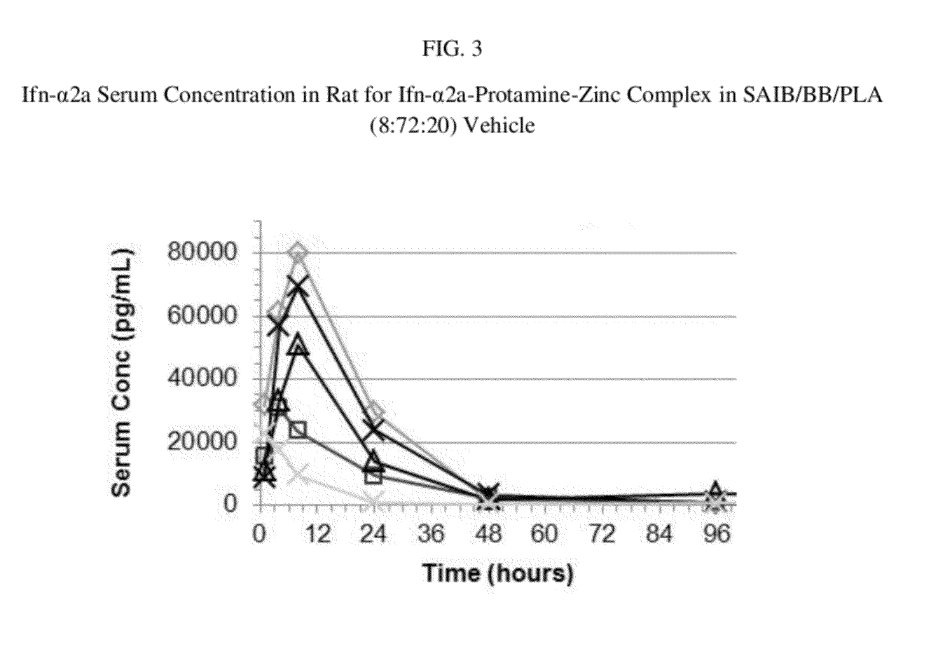

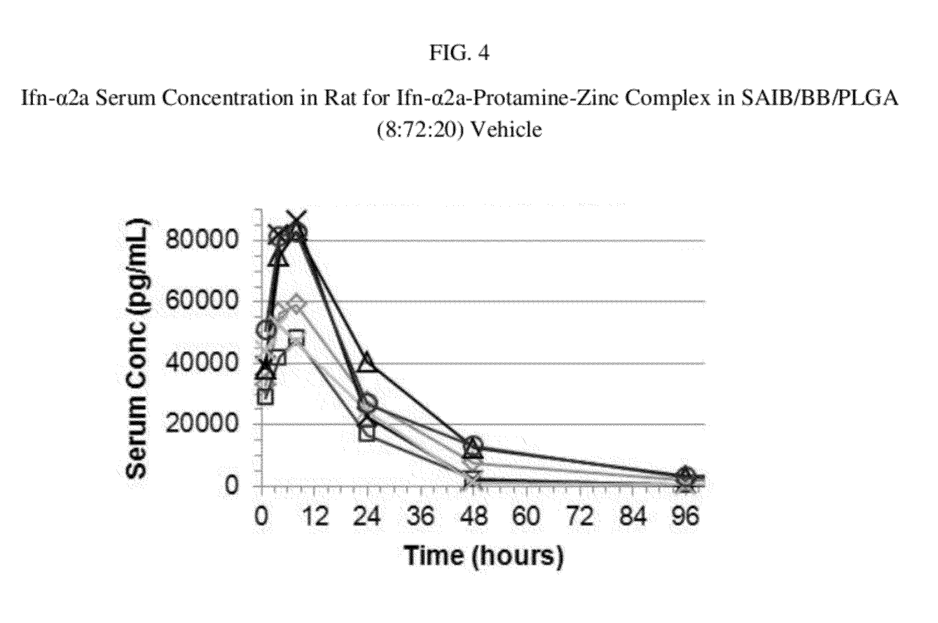

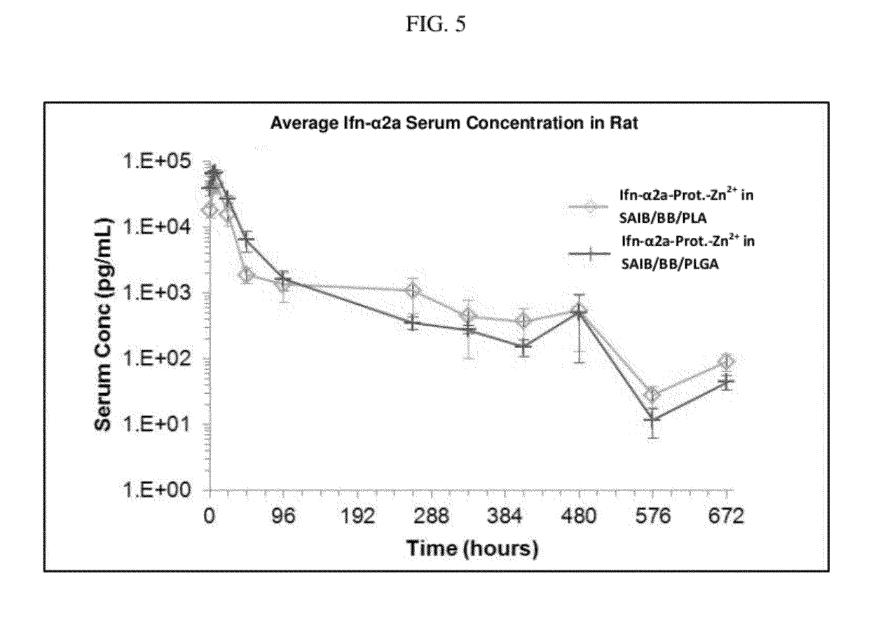

Serum concentrations for each rat in both formulation groups A) and B) were plotted versus time up to 96 hours as shown in FIGS. 3 and 4 respectively. The profiles are similar across formulations. Average serum profiles for the two formulations were nearly identical out to 11 days as depicted in FIG. 5. On average tₘₐₓ was 8 h (range 1-24 h) for both formulations, and Cₘₐₓ ranged from 40−60×10⁴ pg/mL. Serum levels fell ˜50-fold over 11 days and Cₘₐₓ/Cₗₐₛₜ˜500. The formulations studied were similar in their bioavailability (BA) profiles, with BA up to 28 days ranging from 20 to 50%.

### Example 4

### Further In-Vivo Evaluation in Rat for IFNα2a Biodegradable Drug Delivery Depot Formulation

The following formulations were administered via subcutaneous bolus to rats and IFNα2a serum concentration was monitored over time:

C) 20 mg/ml IFNα2a formulation with 1% sucrose and protamine (IFNα2a:protamine 1:0.3 m/m), dispersed in a SAIB/BB/PLA (8:72:20) vehicle; and

D) 20 mg/ml IFNα2a formulation with 1% CMC and 1% sucrose, dispersed in a SAIB/BB/PLA (8:72:20, % w/w) vehicle.

The protein dose was 1 mg for each formulation (500 of 20 mg/ml formulation). Injections were via Excel 1 ml syringes using 23 gauge ⅝ inch Terumo needles.

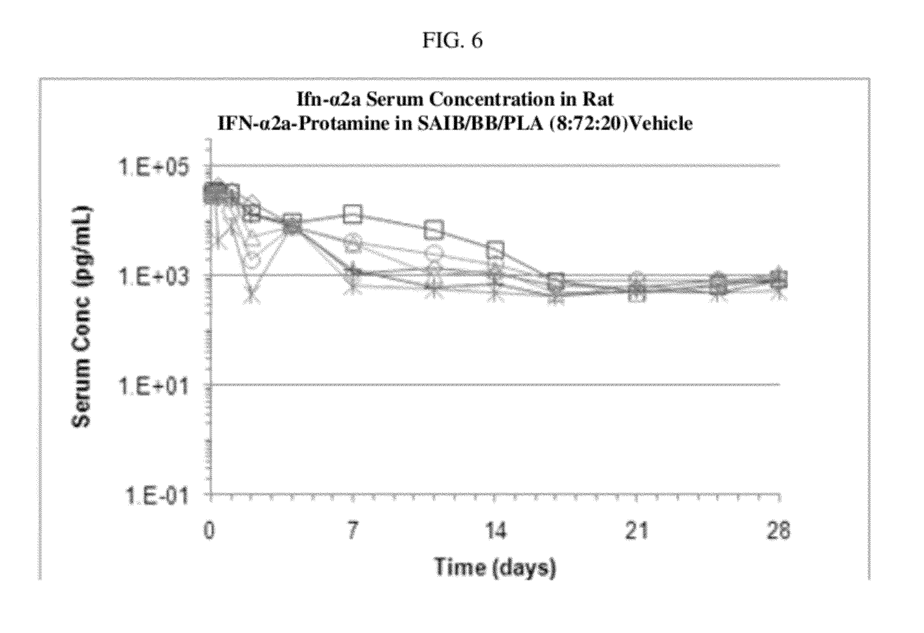

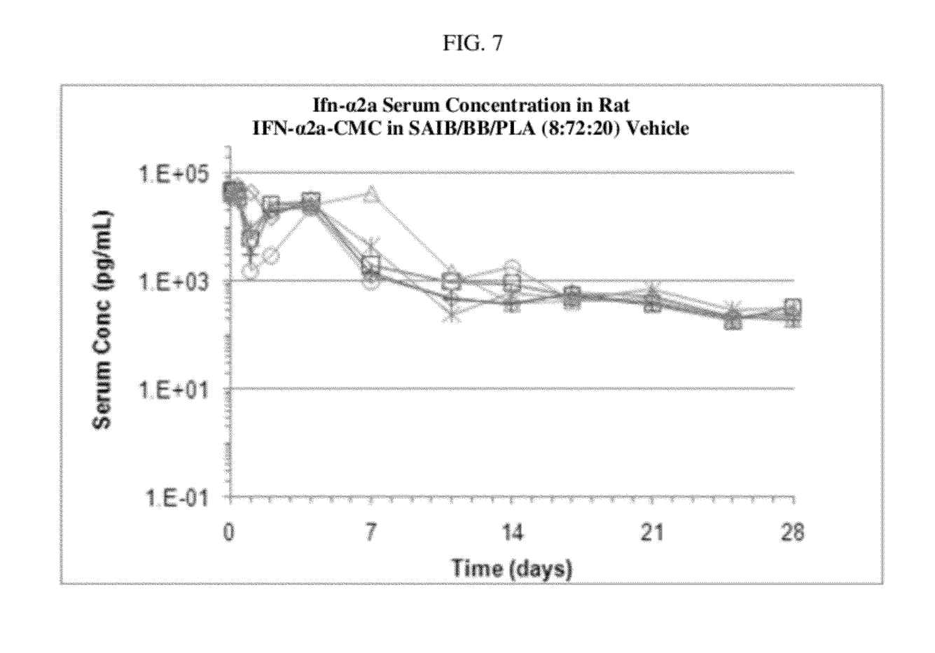

Serum concentrations (as determined by ELISA) for each rat in each formulation group were plotted versus time. The results for formulations C) and D) are provided in FIGS. 6 and 7 respectively. Both formulations demonstrated desirable release kinetics for an injectable depot formulation.

### Example 5

### In-vivo Analysis in Primate for IFNα2a Biodegradable Drug Delivery Depot Formulation

Using depot compositions similar to those above for Example 4, a pharmacokinetic study was performed in primates (cynomolgus monkeys—Macaca fascicularis). Specifically, 2 mg/kg of a 40 mg/ml IFNα2a formulation with 1% sucrose and protamine (IFNα2a:protamine 1:0.3 m/m), dispersed in a SAIB/BB/PLA (8:72:20, % w/w) vehicle was administered to a first group. Another experimental group received 2 mg/kg of a second formulation, 40 mg/ml IFNα2a formulation with 1% CMC and 1% sucrose, dispersed in a SAIB/BB/PLA (8:72:20, % w/w) vehicle. Injections were subcutaneous via Excel 1 ml syringes using 23 gauge ⅝ in Terumo needles.

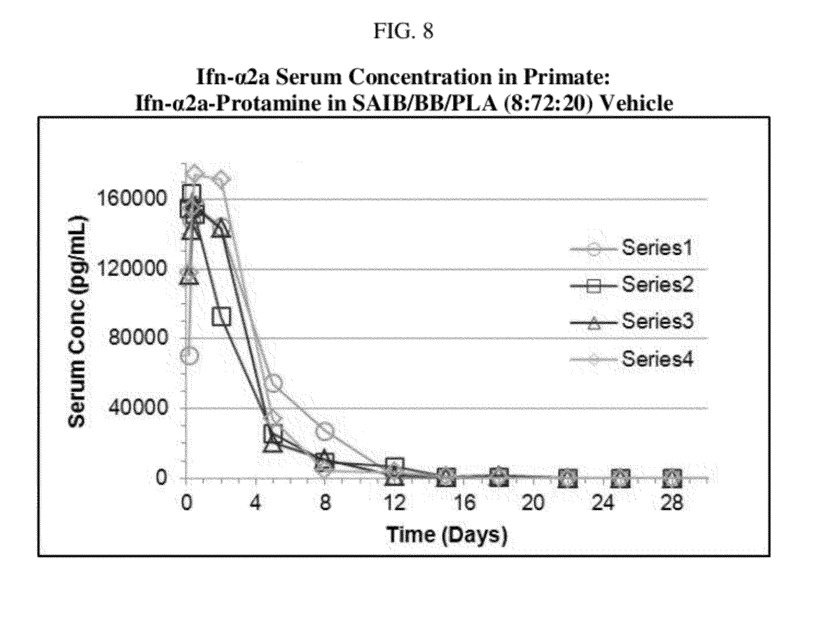

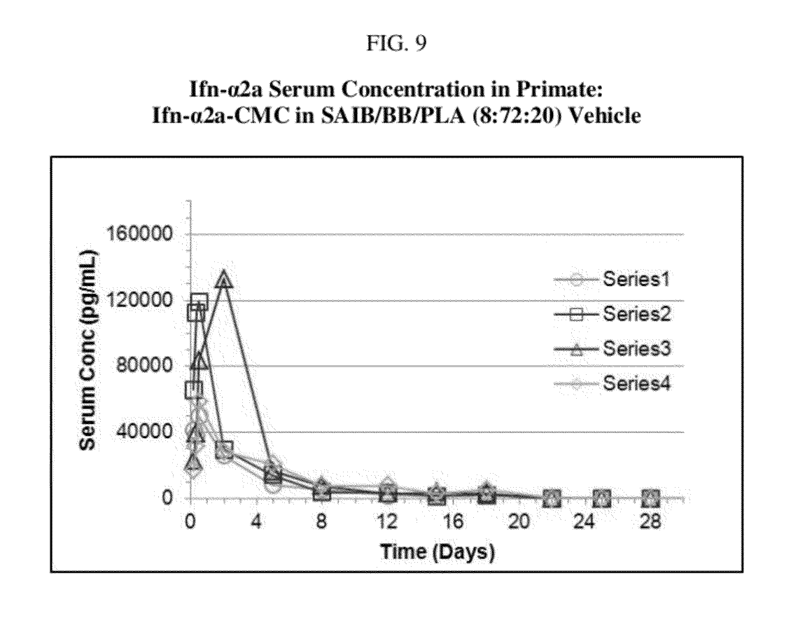

The serum profiles for the individual animals in each group are shown in FIGS. 8 and 9 respectively. As shown, greater serum levels were achieved over the initial 10-12 days with protamine-IFNα2a complex than with CMC-IFNα2a complex.

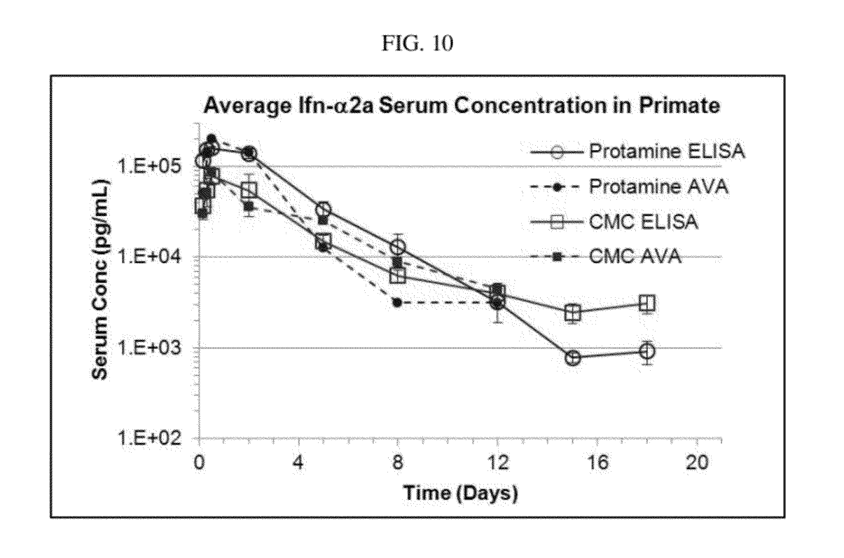

Serum samples from individual animals in each treatment group were analyzed by ELISA and pooled serum samples from each treatment group were analyzed by Anti-Viral Assay (AVA). A comparison of group average serum profiles for the experimental groups as determined by ELISA and AVA is provided in FIG. 10, which reveals that the CMC-complex provided for longer duration of delivery than the protamine complex

### Example 6

### Pharmacokinetic Evaluation of an Anti-Cancer Nucleoside Analogue Delivered from a SAIB/BB/EtOH/PLGA (8/67/5/20) Vehicle

An injectable depot composition was prepared using a protamine complex of an anti-cancer nucleoside analogue pro-drug and SAIB/BB/EtOH/PLGA (8/67/5/20, % w/w) as the vehicle, prepared as follows: 3.3180 g of the nucleoside analogue pro-drug was weighed in a 500 mL glass container. 166 mL of water was added to the glass container and stirred at 400 rpm for 1 hour until all the powder dissolved. The solubility of the nucleoside analogue in water was about 20 mg/mL. The resulting clear aqueous solution was added to 430 mL of a 10 mg/ml protamine sulfate solution. The mixture was stirred again for 1 hour at room temperature for the reaction to complete after which time a white fluffy suspension was formed. The white suspension was distributed in 50 mL plastic tubes. The glass container was rinsed with 65 mL of water and the remaining mixture was transferred to the 50 mL tubes. The tubes containing the suspension were centrifuged at 2500 rpm for 12 min. Following centrifugation, the tubes yielded a total of 547 mL of supernatant and 117 mL of white precipitate.

The supernatant was analyzed via HPLC for free beneficial agent content. The target dosage was 150 mg of beneficial agent. Accordingly, the suspension was aliquoted into 20 10 mL glass vials, each containing 5.8 mL of the white precipitate. The vials containing the precipitate were then lyophilized using an FTS freeze dryer.

The stabilized-beneficial agent complex powder from the 10 mL vials was transferred into 2 mL vials and weighed. Vehicle (SAIB/BB/EtOH/PLGA) (8/67/5/20) was added to the weighed powder to obtain a target concentration of 120 mg/mL of beneficial agent. The mixture was wetted for 1.5 hours with the vehicle, and the wetted mixture was then homogenized for 10 min on a PowerGen 1000 (Fisher Scientific), with probe 5×95 mm to obtain a homogeneous milky white suspension. This suspension was dosed into primates and blood samples were monitored up to 168 hours for both the beneficial agent and a metabolite thereof. Injections were subcutaneous via Excel 1 ml syringes using 23 gauge ⅝ inch Terumo needles. The following dosages were monitored: immediate release formulation (without SAIB/BB/EtOH/PLGA vehicle) at 3 mg/kg; and pro-drug-vehicle compositions at 9 mg/kg, 13.5 mg/kg, and 18 mg/kg.

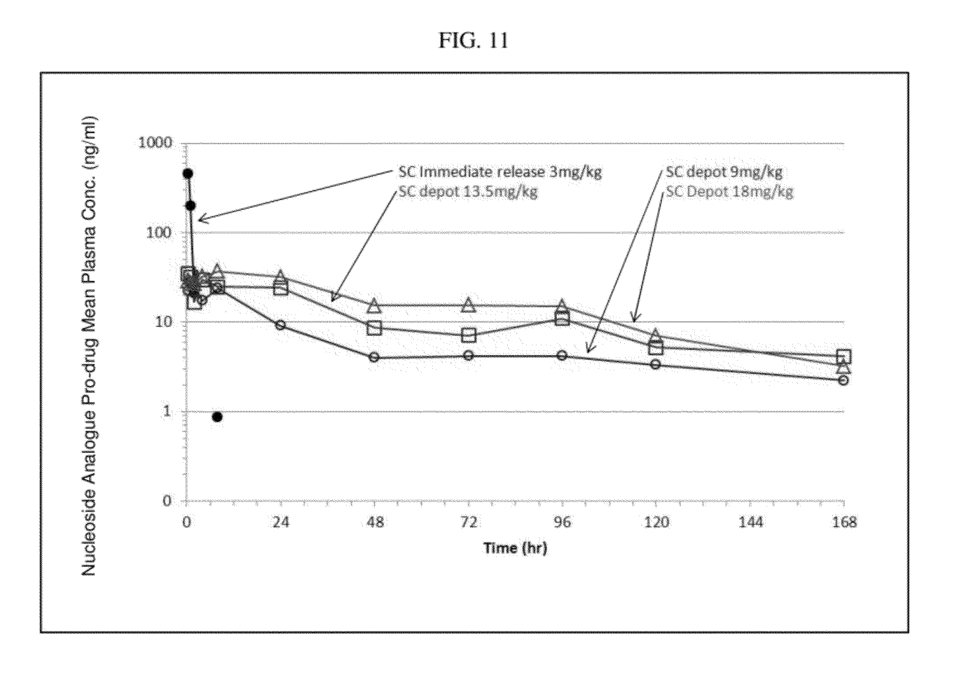

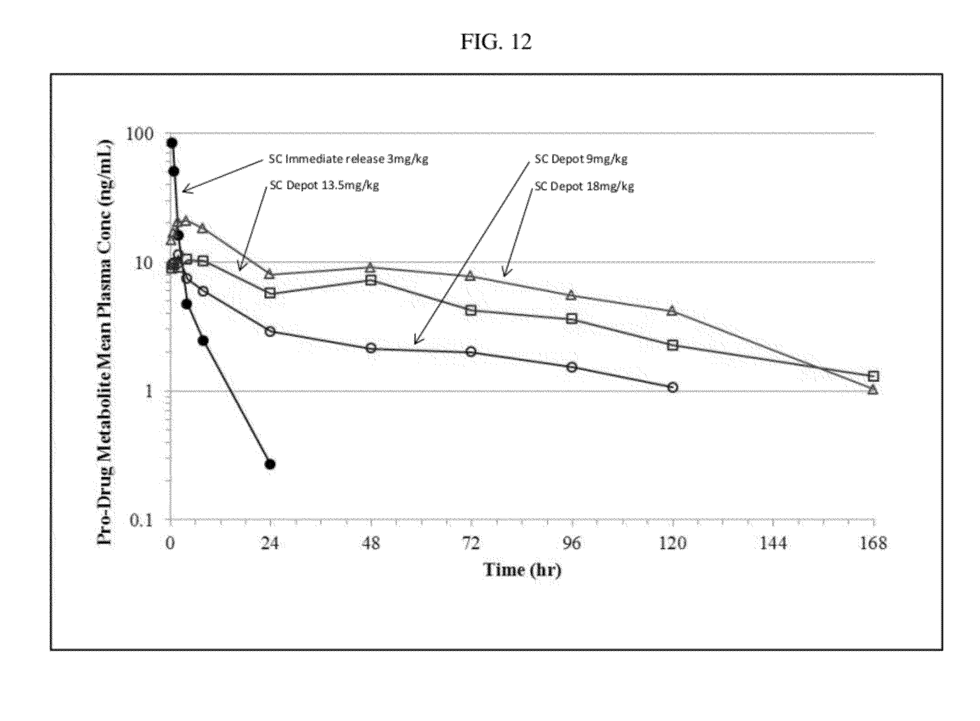

The pharmacokinetic curves for delivery of the nucleoside analogue pro-drug and its active metabolite (the beneficial agent) are provided in FIGS. 11 and 12 respectively. These curves show a desirable delivery profile with low burst effect and sustained release out to 168 hrs.

### Example 7

### Pharmacokinetic Evaluation of a GLP-1 Analogue Delivered from a SAIB/BB/BA/PLA (20/50/10/20) Vehicle

A pharmacokinetic analysis was performed for a Glucagon-like peptide-1 (GLP-1) analogue beneficial agent complexed with zinc and protamine and delivered from a SAIB (sucrose acetate isobutyrate):BB (benzyl benzoate):BA (benzyl alcohol):lactic acid-initiated PLA (polylactic acid) (20/50/10/20, % w/w) vehicle in mini-pig.

GLP-1 analogue complex powder was prepared via spray drying as set forth in Tables 2 and 3 below.

TABLE 2

| | | Volume | Amount |
|---|---|---|---|
| Stock Solution | Concentration | (mL) | (mg) |
| GLP-1 analogue | 100.03 mg/mL | 4.5 | 450.0 |
| peptide Solution | | | |
| Ammonium | 0.396 g in 100 mL water | | 17.8 |
| Bicarbonate | (50 mM) | | |
| Zinc Acetate. 2H2O | 2.194 g in 100 mL water | 2.4 | 52.6 |
| | (100 mM) | | |
| Sucrose Solution | 10 mg in 1 mL water | 7.5 | 75.0 |
| | (300 mM) | | |
| Protamine Sulfate | 10 mg/mL in water | 27.4 | 274 |
| Acetic Acid, glacial | | 2 | |
| | Total | 43.8 | 869.4 |
| % GLP-1 analogue | Expected (theoretical) | | 51.7% |
| peptide(wt/wt) | | | |

TABLE 3
Spray Drying Parameters for Aqueous Condition of Zinc
and Protamine-Stabilized Powder

| | Instrument Setting | Actual Reading |
|---|---|---|
| Inlet temperature | 125° C. (initial) | 113-120° C. |
| Outlet temperature | Controlled by inlet | 76° C. |
| | temperature and liquid | |
| | feed rate | |
| Liquid feed rate | 13% (~4 mL/min) | 13% (~4 mL/min) |
| Atomizing nitrogen flow | 45 mm | 45 mm |
| Aspiration | 100% | 100% |
| Nozzle cleaning | 0 (for suspension 0-2) | 0 (for suspension 0-2) |

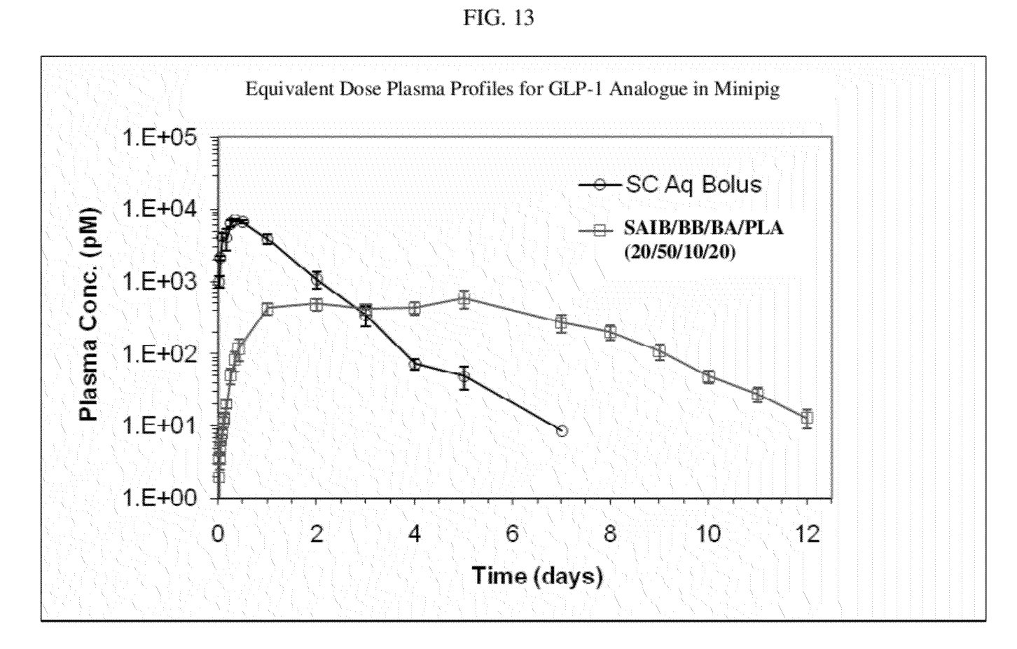

Following spray drying, the GLP-1 analogue complex powder was loaded into 5 mL glass syringes, stoppered and sealed in an aluminum pouch. The syringes were subsequently mixed with 1 mL of vehicle per syringe, SAIB/BB/BA/PLA (20/50/10/20), for use in an in-vivo mini-pig study. Administration was via subcutaneous injection of 600 of 40 mg/ml GLP-1 analogue in vehicle using a Terumo Sursaver syringe with a 25 gauge ½ inch needle. Serum concentration for the GLP-1 analogue was monitored for a period of 12 days post administration. The results of this experiment are shown in FIG. 13 which is a graph of average GLP-1 analogue serum concentration over time. Sustained release of the GLP-1 analogue delivered from the SAIB/BB/BA/PLA vehicle over a 12 day period was demonstrated. The plasma profile resulting from subcutaneous injection of an immediate release formulation of the GLP-1 analogue in aqueous solution is provided in FIG. 13 for comparison.

### Example 8

### Vehicle Viscosity

In vitro vehicle viscosity was determined for the following vehicle material combinations: BB (alone), BB:PLA, SAIB:BB:PLA, SAIB:BB. For each combination of materials, the % w/w of the materials was varied as shown in Table 4 below. Table 4 provides viscosity values in centipoise (cP) for each of the various combinations at 25 and 37° C. without exposure to an aqueous medium. Results for (C) formulations are provided for comparison purposes but are not considered as injectable depot compositions of the present disclosure (D) based on the component % and/or the resulting viscosity.

TABLE 4

| | | | | | Viscosity |
|---|---|---|---|---|---|
| Formulation | SAIB % | BB % | PLA % | Viscosity (cP) | (cP) |
| Type | (w/w) | (w/w) | (w/w) | at 25° C. | at 37° C. |
| (D) | 0 | 98 | 2 | 11.5 | 7.83 |
| (D) | 0 | 96 | 4 | 16.2 | 11 |
| (D) | 0 | 92 | 8 | 31.7 | 20.2 |
| (D) | 0 | 85 | 15 | 100 | 57 |
| (D) | 0 | 70 | 30 | 1130 | 460 |
| (C) | 12 | 88 | 0 | 12.0 | 8 |
| (D) | 12 | 73 | 15 | 172 | 90 |
| (C) | 12 | 58 | 30 | 2600 | 890 |
| (C) | 24 | 76 | 0 | 18.0 | 5.68 |
| (D) | 24 | 61 | 15 | 326 | 150 |
| (C) | 24 | 46 | 30 | 7130 | 1900 |
| (C) | 36 | 64 | 0 | 33.0 | N/A |
| (C) | 36 | 49 | 15 | 798 | 296 |

Table 4 demonstrates that vehicle compositions of the present disclosure, e.g., vehicle compositions including a biodegradable polymer (here poly lactic acid—PLA) present in an amount of from about 5% to about 30% by weight of the vehicle and a hydrophobic solvent (here benzyl benzoate—BB) present in an amount of from about 95% to about 70% by weight of the vehicle have viscosity values of less than 1,200 centipoise at both 25 and 37° C.

In-situ viscosity measurements were also performed which demonstrate the viscosity changes in selected vehicle compositions over time, during exposure to an aqueous medium. These results are provided in Table 5 below with values being provided for both low and high shear rates. Viscosity was measured following injection of 1.5 mL of the vehicle into 100 mL of phosphate buffered saline (PBS) at pH 7.4 and 37° C.

TABLE 5

| | Excipient | | Viscosity (cP) | | | | | |
|---|---|---|---|---|---|---|---|---|
| Sample ID | %, w/w | | T₀ | 5 hrs | 24 hrs | 48 hrs | 72 hrs | 168 hr. |
| SAIB/BB/PLA | 8/72/20 | (low shear | 158 | 143 | 143 | 134 | 131 | 130 |
| (BI), | | rate) | | | | | | |
| 14.2 kD | | (0.6/sec) | | | | | | |
| | | (high shear | 147 | 128 | 130 | 126 | 126 | 116 |
| | | rate) (20/sec) | | | | | | |
| SAIB/BB/PLA | 8/72/20 | (low shear | 141 | 148 | 146 | 132 | 134 | 120 |
| (DURECT) | | rate) | | | | | | |
| 14.5 kD | | (0.6/sec) | | | | | | |
| | | (high shear | 140 | 133 | 131 | 125 | 128 | 119 |
| | | rate) (20/sec) | | | | | | |
| SAIB/BB/PεCGL | 8/72/20 | (low shear | 200 | 192 | 202 | 197 | 203 | 213 |
| (ter- | | rate) | | | | | | |
| Polymer) | | (0.6/sec) | | | | | | |
| 27 kD | | (high shear | 207 | 197 | 194 | 192 | 201 | 203 |
| | | rate) (20/sec) | | | | | | |
| SAIB/BB/EtOH/PLA | 8/67/5/20 | (low shear | 267* | 160 | 161 | 159 | 161 | 144 |
| (BI) 14.2 kD | | rate) | | | | | | |
| | | (0.6/sec) | | | | | | |
| | | (high shear | 95 | 153 | 150 | 151 | 152 | 137 |
| | | rate) (20/sec) | | | | | | |
| SAIB/BB/EtOH/PLA | 8/67/5/20 | (low shear | 85 | 165 | 165 | 156 | 162 | 150 |
| (Lactel) | | rate) | | | | | | |
| 14.5 kD | | (0.6/sec) | | | | | | |
| | | (high shear | 85 | 148 | 154 | 151 | 151 | 140 |
| | | rate) (20/sec) | | | | | | |

*Note: This value may be inaccurate.

The inclusion of some ethanol in the vehicles containing LA-initiated PLA is believed to be responsible for the increase in viscosity after the exposure to PBS buffer at 37° C. observed for some of the vehicles. However, the viscosity of individual vehicles after exposure to water remained relatively constant over the test period up to 168 hours regardless of the composition of the vehicles, confirming that any rate controlling surface “cloud” layer, formed upon exposure of the formulations to PBS buffer, at the surface of the formulation and PBS buffer does not have physical strength or appreciable mechanical structures resisting the applied shear stress at the range of shear rates indicated in Table 5. This may be contrasted with gel forming vehicles which exhibit a substantial increase in viscosity over time when exposed to an aqueous environment.

Additional in-situ viscosity measurements are provided in Table 6 below. Depending on the observed viscosity of the formulations, an appropriate Brookfield viscometer model was selected in order to match the required (or optimum) range of torque. For example, a Brookfield viscometer model DV-III+ULTRA (HA) model was used to provide low shear rates of 140-320 sec⁻¹ at 25° C. and high shear rates of 500 sec⁻¹ at 25° C.; a Brookfield DV-111+ULTRA (LV) model was used to provide low shear rates of 7-28 sec⁻¹ at 25° C. and high shear rates of 40-200 sec⁻¹ at 25° C.; a Brookfield DV-III+(HB) model was used to provide low shear rates of 370-500 sec⁻¹ at 37° C. and high shear rates of 500 sec⁻¹ at 37° C.; and a Brookfield DV-III+(LV) model was used to provide low shear rates of 20-46 sec⁻¹ at 37° C. and high shear rates of 90-350 sec⁻¹ at 37° C. Viscosity was measured following injection of 1.5 mL of the vehicle into 100 mL of phosphate buffered saline (PBS) at pH 7.4

TABLE 6

| | | | Solution | Solution and In-Situ η (cP) at 37 C. | | | | |
|---|---|---|---|---|---|---|---|---|
| | Relative | Solution η | η (cP) at | | T = 5 | T = 24 | T = 48 | |
| Formulation | Shear Rate | (cP) at 15 C. | 25 C. | T = 0 | hours | hours | Hours | T = 7 days |
| SAIB/EtOH/BA/PLA | Low Shear | 7719 | 2269 | 781.2 | 10138 | 35719 | 80037 | 121099 |
| (79/10/1/10) | High Shear | 7083 | 2282 | 781.2 | 9389 | 32901 | 73513 | 110862 |
| SAIB/BB/BA/PLA | Low Shear | 856.9 | 434.0 | 192.2 | 265.7 | 327.0 | 368.9 | 341.9 |
| (20/50/10/20) | High Shear | 852.5 | | 195.7 | 263.8 | | 335.1 | 342.9 |
| SAIB/NMP/BA/PLA | Low Shear | | 872.8 | 329.5 | 2653 | 7336 | 10081 | 27441 |
| (65/15/10/10) | High Shear | | | 334.8 | 2502 | 6184 | 8927 | 23074 |
| SAIB/NMP/EtOH/PLA | Low Shear | 1999 | 621.6 | 272.4 | 49069 | 174344 | 236540 | 431805 |
| (55/10/15/20) | High Shear | 2032 | | 269.1 | 24382 | 80646 | 119858 | 302350 |
| SAIB/BB/BA/PLA | Low Shear | 132.5 | 71.3 | 39.99 | 48.93 | 55.80 | 61.0 | 59.7 |
| (20/60/10/10) | High Shear | 133.9 | 71.53 | 39.90 | 48.30 | 54.04 | 59.5 | 58.4 |
| SAIB/NMP/EtOH/PLA | Low Shear | 71.5, 78.1 | 45.8, 51.6 | 30.1, 31.9 | 599289 | 3671140 | 5675384 | 2262216 |
| (20/50/10/20) | High Shear | 73.4, 77.9 | 47.5, 51.0 | 31, 31.9 | 387884 | 2288218 | 3079789 | 980294 |
| SAIB/DMSO/PLA | Low Shear | | 169.0 | 92.0 | 3783589 | | 5199128 | |
| (30/50/20) | High Shear | | | 92.0 | | | 3078953 | |

The vehicles described in Table 6 fall into two categories, those composed of solvents EtOH and NMP both of which elute readily into the external aqueous medium, and those containing the hydrophobic solvent BB, which elutes extremely slowly, and BA, which elutes at an intermediate rate. As shown in Table 6, for vehicles comprising hydrophilic solvents, the in situ viscosity increases several Logs over 7 days, mostly in the first 5 hours of exposure to aqueous medium. In situ viscosities for the BB/BA vehicles do not exhibit this level of viscosity increase and instead exhibit relatively stable viscosity over time.

Additional in-situ viscosity measurements are provided in Table 7 below which compares carriers having only BB as the solvent with carriers including BB and a secondary hydrophobic solvent, e.g., BA (benzyl alcohol) or TA (triacetin). In situ viscosity was measured as described above for Table 6.

TABLE 7

| | | | | | Brookfield | Brookfield | Brookfield | Brookfield |
|---|---|---|---|---|---|---|---|---|
| | | | | | model | model | model | model |
| | | | | | DV-III + | DV-III + | DV-III + | DV-III + |
| | | | | | ULTRA | ULTRA | ULTRA | ULTRA |
| | | | | | (HA) | (HA) | (HA) | (HA) |
| | | Shear | η (cP) | η (cP) | η (cP) | η (cP) | η (cP) | η (cP) |
| Sample ID | Composition | Rate | 25° C. | 37° C. | 5 h | 24 h | 48 h | 120 h |
| 1248-124-1 | SAIB:BB:la- | Low | 328 | 146 | 186 | 169 | 140 | 128 |
| | PLA, 8:72:20 | High | 320 | 145 | 176 | 167 | 141 | 128 |
| 1248-123-1 | SAIB:BB:dd- | Low | 235 | 116 | 109 | 135 | 118 | 115 |
| | PLA, 8:72:20 | High | 236 | 115 | N/A | 136 | 116 | 114 |
| 1248-124-6 | SAIB:BB:BA:la- | Low | 161 | 89.2 | 167 | 206 | 169 | 198 |
| | PLA, 8:52:20:20 | High | 161 | 86.0 | 164 | 208 | 168 | 198 |
| 1248-124-3 | SAIB:BB:BA:dd- | Low | 129 | 71.3 | 89.0 | 144 | 165 | 152 |
| | PLA, 8:52:20:20 | High | 129 | 69.7 | N/A | 142 | 161 | 151 |
| 1248-125-7 | SAIB:BB:TA:la- | Low | 391 | 179 | 206 | 218 | 227 | 226 |
| | PLA, 8:52:20:20 | High | 387 | 178 | 205 | 219 | 227 | 225 |
| 1248-124-5 | SAIB:BB:TA:dd- | Low | 298 | 132 | 168 | 155 | 168 | N/A |
| | PLA, 8:52:20:20 | High | 294 | 135 | 163 | 153.2 | 165.1 | N/A |

Generally the vehicles containing only BB as the solvent showed relatively stable viscosity for a period up to 120 hours at 37° C. The vehicles containing BB and BA showed an increase in viscosity of about 2× over the 120 hour time period at 37° C. Finally, the vehicle containing BB and TA showed a slight increase in viscosity (about 50%) over the 120 hour time period at 37° C. However, even for those vehicles showing an increase in viscosity, viscosity remained relatively low, e.g., less than 500 cP over the 120 hour time period.

Table 8 below provides in vitro viscosity (cP) measurements for two SAIB:BB:PLA (8:72:20) vehicles and a SAIB:BB:PεCGL (8:72:20) vehicle over a range of temperatures. The viscosity values for 25° C. (298° K) and 37° C. (310° K) are indicated in bold.

TABLE 8

| SAIB:BB:PLA (BI) 8:72:20 | | | SAIB:BB:PLA (Lactel) 8:72:20 | | | SAIB:BB:PεCGL 8:72:20 | | |
|---|---|---|---|---|---|---|---|---|
| | | High | | | High | | | High |
| Temp °K | Low Shear | Shear | Temp °K | Low Shear | Shear | Temp °K | Low Shear | Shear |
| 258 | 28002 | 27947 | 258 | 36570 | 29982 | 258 | 25676 | 21752 |
| 263 | 10824 | 10802 | 263 | 14241 | 11949 | 263 | 10087 | 10018 |
| 268 | 5040 | 5050 | 268 | 5279 | 5279 | 268 | 5292 | 5249 |
| 273 | 2569 | 2564 | 273 | 2602 | 2610 | 273 | 2942 | 2941 |
| 278 | 1354 | 1395 | 278 | 1447 | 1481 | 278 | 1817 | 1838 |
| 283 | 819.6 | 831.1 | 283 | 830.6 | 842 | 283 | 1139 | 1103 |
| 288 | 525.4 | 533.6 | 288 | 531.4 | 538.8 | 288 | 767.2 | 774.7 |
| 293 | 230.7 | 229 | 293 | 355.6 | 355.1 | 293 | 575.7 | 539.8 |
| 298 | 158 | 155.1 | 298 | 240 | 245 | 298 | 403 | 399.9 |
| 310 | 71 | 73.6 | 310 | 118.5 | 115.9 | 310 | 196.8 | 203.9 |

Table 8 demonstrates that each of the above vehicles has relatively low in vitro viscosity, e.g., less than 500 cP at both 25° C. and 37° C.

Table 9 provided below provides in vitro viscosity measurements for additional vehicles at 25° C. and 37° C. The vehicles are as follows: BA:dd-PLGA, 333-44-1, 6.7 kDa, dodecanol-initiated, 65:35 L:G; BA:ga-PLGA, 11.5 kDa, glycolate-initiated, 64:36 L:G; EB:dd-PLGA (ethyl benzoate); EB:ga-PLGA; TA:dd-PeCL (triacetin), 14.2 kDa, dodecanol-initiated 20:80 C:L; TA:la-PeCL, and 14.8 kDa, lactate-initiated, 20:80 C:L.

All vehicles were 80:20 (% w/w) solvent:polymer. BA=benzyl alcohol; EB=ethyl benzoate; and TA=triacetin; N/A=not available.

TABLE 9

| | | Brookfield | | Brookfield | |
|---|---|---|---|---|---|
| | | model | Brookfield | model | |
| | | DV-III + | model | DV-III + | Brookfield |
| | | ULTRA | DV- | ULTRA | model DV- |
| | Shear | (HA) | III + (LV) | (HA) | III + (LV) |
| SAMPLE ID | Rate | 25° C. | 25° C. | 37° C. | 37° C. |
| BA:dd-PLGA | Low | 30.16 | 30.82 | 35.72 | NA |
| | High | N/A | 30.21 | N/A | NA |
| BA:ga-PLGA | Low | 59.53 | 58.38 | 34.13 | 35.46 |
| | High | N/A | 57.05 | N/A | 34.14 |
| EB:dd-PLGA | Low | 15.88 | 15.01 | 10.32 | 10.23 |
| | High | N/A | 14.95 | N/A | 9.86 |
| EB:ga-PLGA | Low | 33.34 | 32.95 | 19.84 | 19.78 |
| | High | N/A | 32.16 | N/A | 19.72 |
| TA:dd-PεCL | Low | 266.1 | 269.7 | 118 | 116.2 |
| | High | 262.7 | 255.4 | 117.5 | 114.9 |
| TA:la-PεCL | Low | 360.2 | 350.3 | 154.9 | 160.8 |
| | High | 354.8 | 347.9 | 154 | 156 |

Table 9 demonstrates that each of the above vehicles has relatively low in vitro viscosity, e.g., less than 500 cP at both 25° C. and 37° C.

### Example 9

### Injectability Study: SAIB/BB/EtOH/PLGA

Injectability data and test conditions are presented in Table 10. The formulation was made up of the 120 mg/ml load of nucleoside analogue pro-drug lyophilized with protamine complex which was dispersed in a SAIB/BB/EtOH/PLGA (8/67/5/20, % w/w) vehicle. The injectable depot composition was prepared as described previously in Example 6.

The suspension was tested for injectability by backfilling 100 μL suspension into 1 mL syringe with permanently attached needle 21G or 23G×½″ (Terumo REF SS01D2313). A force of 10 lbs was applied to the syringe and injection times were monitored both with and without a delay following mixing. Temperature was 25° C.

The injection times (less than 2 seconds for 0.21-0.25 ml) were deemed acceptable for the nucleoside analogue complex formulation using both 21G and 23G×1 inch needles.

TABLE 10

| | | Wait | | | | | |
|---|---|---|---|---|---|---|---|
| | Vehicle | after | | | | | |
| Pro-Drug | Volume | Mixing | Force | Temp | Needle | Volume | Time |
| Load (mg) | (mL) | (h) | (lbs) | ° C. | Gauge | (mL) | (s) |
| 120 | 1 | 0 | 10 | 22.9 | 21 | 0.21 | 1.4 |
| | | | | | | 0.23 | 1.5 |
| | | | | | | 0.24 | 1.6 |
| | | | | | | 0.24 | 1.6 |
| 120 | 1 | 0 | 10 | 22.9 | 23 | 0.23 | 1.6 |
| | | | | | | 0.25 | 1.7 |
| | | | | | | 0.21 | 1.6 |
| 120 | 1 | 1 | 10 | 22.9 | 21 | 0.23 | 1.6 |
| | | | | | | 0.22 | 1.5 |
| 120 | 1 | 1 | 10 | 22.9 | 23 | 0.21 | 1.4 |
| | | | | | | 0.23 | 1.5 |
| 120 | 1 | 4 | 10 | 22.9 | 21 | 0.24 | 1.6 |
| | | | | | | 0.21 | 1.5 |
| 120 | 1 | 4 | 10 | 22.9 | 23 | 0.22 | 1.5 |
| 120 | 1 | 6 | 10 | 22.9 | 21 | 0.22 | 1.5 |
| | | | | | | 0.25 | 1.7 |
| 120 | 1 | 6 | 10 | 22.9 | 23 | 0.25 | 1.7 |

### Example 10

### Injectability Study: SAIB/BB/PLA (8/72/20)

An additional injectability study was conducted using a GLP-1 analogue as the beneficial agent complexed with Zinc and protamine and dispersed in a SAIB/BB/PLA vehicle. The test conditions and results are provided below in Table 11. A force of 10 lb was applied to a 1 ml EXEL syringe using either a 25 or 27 gauge needle and injection times were monitored.

TABLE 11

| | | Complex | | | | | | |
|---|---|---|---|---|---|---|---|---|
| | Beneficial | Peptide/Zn/ | | | | | | |
| | agent Load | Protamine | Vehicle | Force | Temp | Needle size | Volume | Time |
| Formulation | (mg) | (mol ratio) | (mL) | (lbf) | (° C.) | (G = gauge) | (mL) | (sec) |
| Beneficial | 70 | 1:0.4:0.3 | 1 | 10 | 22.6 | 25G ⅝″, | 0.51 | 21.6 |
| Agent | | | | | | UTW | | |
| Complex + | | | | | 21.9 | 25G ⅝″, | 0.51 | 21.1 |
| SAIB/BB/PLA | | | | | | UTW | | |
| (8/72/20, % | | | | | 22.1 | 25G ⅝″, | 0.51 | 19.3 |
| w/w) | | | | | | UTW | | |
| | | | | 10 | 19.9 | 27G ½″, | Stopped in | |
| | | | | | | UTW | between | |
| | | | | | 19.9 | 27G ½″, | | |
| | | | | | | UTW | | |
| Beneficial | 70 | 1:0.4:0.3 | 1 | 10 | 22.2 | 27G ½″, | 0.52 | 12.7 |
| Agent | | | | | | UTW | | |
| Complex + | | | | | 22.5 | 27G ½″, | 0.52 | 11.9 |
| SAIB/BB/PLA | | | | | | UTW | | |
| (8/72/20, % | | | | | 22.0 | 27G ½″, | 0.52 | 12.5 |
| w/w) | | | | | | UTW | | |
| | | | | 10 | 22.5 | 27G ½″, | 0.51 | 17.8 |
| | | | | | | TW | | |
| | | | | | 22.5 | 27G ½″, | 0.51 | 19.4 |
| | | | | | | TW | | |
| | | | | | 22.5 | 27G ½″, | 0.51 | 16.8 |
| | | | | | | TW | | |

The above injection times were deemed acceptable for the GLP-1 analogue formulation when injected using both 25 and 27 gauge needles at approximately 25° C.

### Example 11

### Further In-Vivo Depot Characterization Using rhGH as Beneficial Agent: Sensitivity of Controlled Release to Polymer Characteristics

In order to further characterize the injectable depot composition of the present disclosure additional experiments were conducted using rhGH as the beneficial agent. The experimental design included the testing of 10 different formulations in Sprague Dawley rats. The 10 formulations are described generally in Table 12 and in greater detail below.

TABLE 12

| Beneficial | | Beneficial | |
|---|---|---|---|
| Agent | | Agent or | |
| or Beneficial | | Beneficial Agent | |
| Agent Complex | Vehicle | Complex | Vehicle |
| Free rhGH, | Benzyl Benzoate | rhGH:protamine | Benzyl Benzoate |
| 50 mg/mL | (BB) alone | 2:1 (m/m), | (BB) alone |
| | BB + polymer | 50 mg/mL protein | BB + polymer |
| | (80:20% w/w) | | (80:20% w/w) |
| | 15.2 kDa, lactate- | | 15.2 kDa, lactate- |
| | initiated PLA | | initiated PLA |
| | BB + polymer | | BB + polymer |
| | (80:20% w/w) | | (80:20% w/w) |
| | 13.9 kDa, | | 13.9 kDa, |
| | dodecanol- | | dodecanol- |
| | initiated PLA | | initiated PLA |
| | BB + polymer | | BB + polymer |
| | (80:20% w/w) | | (80:20% w/w) |
| | 11.5 kDa, | | 11.5 kDa, |
| | glycolate- | | glycolate- |
| | initiated, | | initiated, |
| | 64:36 PLGA | | 64:36 PLGA |
| | BB + polymer | | BB + polymer |
| | (80:20% w/w) | | (80:20% w/w) |
| | 6.7 kDa, | | 6.7 kDa, |
| | dodecanol- | | dodecanol- |
| | initiated, 65:35 | | initiated, 65:35 |
| | PLGA | | PLGA |

Formulations

Formulation #1; Identity: rhGH Formulation 1; Description/Physical appearance: Suspension; 50 mg of hGH in 1 mL of Benzyl Benzoate (BB); Storage conditions: 2-8° C.

Formulation #2; Identity: rhGH Formulation 2; Description/Physical appearance: Suspension; 50 mg of hGH in 1 mL of BB:PLA₁, (80:20); Storage conditions: 2-8° C.

Formulation #3; Identity: rhGH Formulation 3; Description/Physical appearance: Suspension; 50 mg of hGH in 1 mL of BB:PLA₂ (80:20); Storage conditions: 2-8° C.

Formulation #4; Identity: rhGH Formulation 4; Description/Physical appearance: Suspension; 50 mg of hGH in 1 mL of BB:PLGA₁, (80:20); Storage conditions: 2-8° C.

Formulation #5; Identity: rhGH Formulation 5; Description/Physical appearance: Suspension; 50 mg of hGH in 1 mL of BB:PLGA₂, (80:20); Storage conditions: 2-8° C.

Formulation #6; Identity: rhGH:protamine Formulation 6; Description/Physical appearance: Suspension; 50 mg of hGH+Protamine in 1 mL of BB+methionine; Storage conditions: 2-8° C.

Formulation #7; Identity: rhGH:protamine Formulation 7; Description/Physical appearance: Suspension; 50 mg of hGH+Protamine in 1 mL of BB:PLA₁+methionine; Storage conditions: 2-8° C.

Formulation #8; Identity: rhGH:protamine Formulation 8; Description/Physical appearance: Suspension; 50 mg of hGH+Protamine in 1 mL of BB:PLA₂, (80:20)+methionine; Storage conditions: 2-8° C.

Formulation #9; Identity: rhGH:protamine Formulation 9; Description/Physical appearance: Suspension; 50 mg of hGH+Protamine in 1 mL of BB:PLGA₁, (80:20)+methionine; Storage conditions: 2-8° C.

Formulation #10; Identity: rhGH:protamine Formulation 10; Description/Physical appearance: Suspension; 50 mg of hGH+Protamine in 1 mL of BB:PLGA₂, (80:20); Storage conditions: 2-8° C.

Abbreviations: BB=Benzyl Benzoate; PLA₁=Poly lactic Acid (lactic acid initiated, M_{w}=15.1 Kd); PLA₂=Poly lactic Acid (dodecanol initiated, M_{w}=13.9 Kd); PLGA₁=Poly lactide-co-glycolide (glycolate initiated (64:36), M_{w}=11.5 Kd; PLGA₂=Poly lactide-co-glycolide (dodecanol initiated (65:35), M_{w}=6.5 Kd. M_{w} is the weight average molecular weight as measured by gel permeation chromatography.

Dose Preparation and Protocol (Test Articles 1-10)

Foil pouches containing 5 mL glass syringes containing the rhGH or rhGH complex in dry form were placed in a clean, dry area at room temperature for a minimum of 60 minutes prior to opening. Diluent vials containing the vehicle were placed in a clean, dry area at room temperature prior to opening. After the foil pouches were allowed to equilibrate at room temperature for 60 minutes, each pouch was opened with a pair of clean scissors. The correct volume of diluent for each formulation (1.0 mL) was withdrawn with a 3 mL syringe (BD PN309585 or equivalent) fitted with a 16 Ga 1 inch needle (BD PN305197 or equivalent). The plastic tip was removed from each 5 mL glass syringe containing the test article powder. One side of a sterile female-female luer adaptor was affixed to each glass syringe. The 3 mL syringe containing the diluent was then connected to the other side of the sterile female-female luer. The total liquid contents of the 3 mL syringe were pushed into the powder contents of the 5 mL glass syringe through the female-female luer. The connected syringes were then left for at least 15 min to wet the powder with the liquid. The liquid was then mixed with the powder by passing the mixture between the two syringes until a uniform suspension was produced (approximately 50 passes between syringes). The total contents of both syringes were then pushed into the 1 mL plastic syringe, and the 1 mL plastic syringe was labeled to identify the lot # and solution. The female-female luer was then removed from the 1 mL plastic syringe. Finally, a 21 Ga 1 inch needle was placed into the luer lock of the 1 mL syringe and the needle was primed with test article suspension.

The above formulations were injected SC as a single dose of 5 mg/rat with an administered volume of 100 μl. The study included 10 groups with 6 rats/group. For groups 1-5, blood was collected from the jugular vein at: Pre-dose (−24 hr), 0.5, 1, 2, 4, 8 and 12 hours; and 1, 2, 3, and 5 days post dose. For groups 6-10, blood was collected from the jugular vein at: Pre-dose (−24 hr), 1, 4, 8, and 12 hours; and 1, 2, 3, 5 and 7 days post dose.

Results

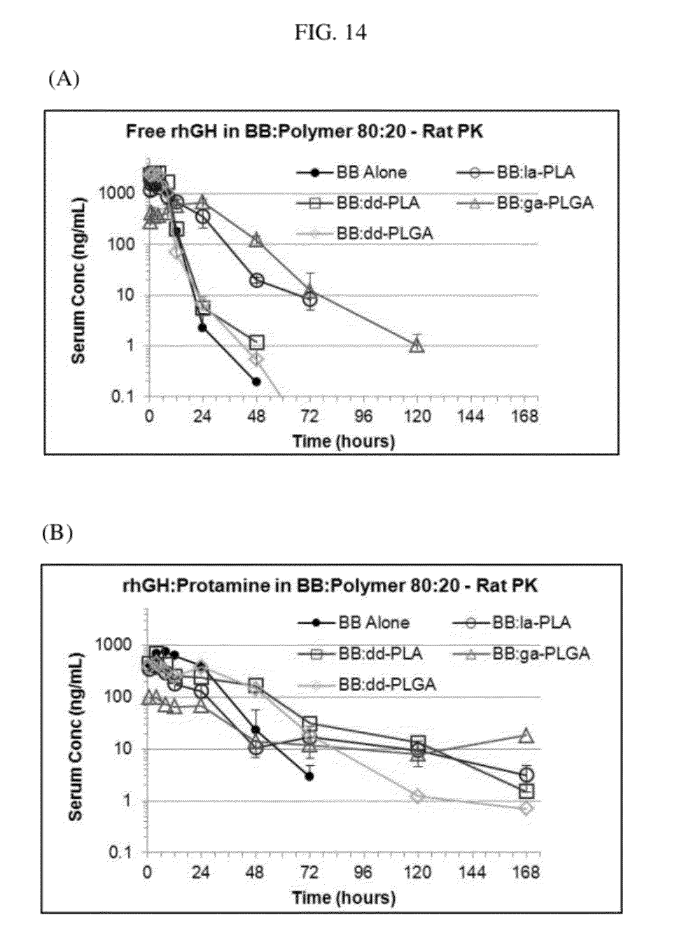

Serum profiles for the above study are provided in FIG. 14 panels A and B. Panel A shows the serum concentration over a 5 day period for free rhGH in the 5 vehicles tested. Panel B shows the serum concentration over a 7 day period for the rhGH:Protamine 0.5:1 (m/m) complex in the 5 vehicles tested. As shown in panel A, for free rhGH, the dodecanol-initiated polymers showed little difference in PK characteristics relative to BB alone. The lactic acid- and glycolic acid-initiated polymers showed lower initial burst and extended delivery relative to BB alone, with the glycolic acid-initiated PLGA providing greater control over release than the lactic acid-initiated PLA.

As shown in panel B for the rhGH:protamine formulations, each of the test vehicles displayed reduced initial release and prolonged duration of delivery relative to the formulations in which free rhGH was dispersed. In particular, delivery was extended even further in the two formulations utilizing the acid-initiated polymers. Note that the use of the rhGH:protamine complex largely compensated for the poorer intrinsic release control demonstrated by the dodecanol-initiated polymers in panel A.

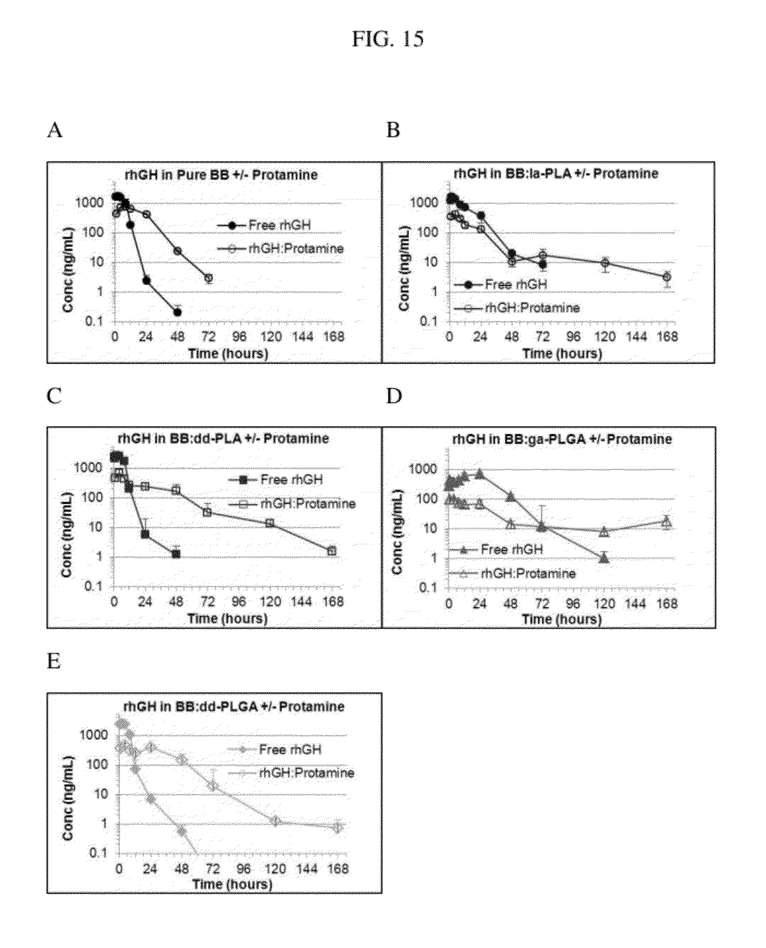

FIG. 15, panels A-E show within formulation comparisons of serum profiles with free vs. complexed rhGH. As shown, complexation with protamine reduced 1 h serum levels ˜2.5 to 8 fold and extended delivery in all cases.

Mean residence time (MRT) is indicative of the duration of delivery. Several processes contribute to MRT including dissolution, transport, absorption and PK. Using the data derived from the above experiment, the separate contributions of polymer and complex to MRT were extracted in order to determine whether the individual effects of protamine complex and polymer on the MRT of free rhGH in BB alone (ΔMRT_{complex} and ΔMRT_{polymer}, respectively) predict their combined effect. An additive model for MRT would be as follows:

MRT_{complex+polymer}=MRT_{BB}+ΔMRT_{complex}+ΔMRT_{polymer}

TABLE 13

| | | | | | | | Fractional Contribution of | | | |
|---|---|---|---|---|---|---|---|---|---|---|
| | | | | ΔMRT | ΔMRT | Additive | Each Component | | | |
| Test | Form of | Contributions | MRT & | Polymer | Complex | Model for | BB | | | |
| Article | rhGH | to MRT | SEM (h) | (h) | (h) | MRT (h) | Alone | Polymer | Complex | Synergy |
| BB | Free/ | Disso + Trans + | 5.41 | | | | | | | |
| Alone | Susp | Abs + PK | 0.68 | | | | | | | |
| BB: la- | Free/ | Disso + Trans + | 12.8 | 7.42 | | | 0.42 | 0.58 | | |
| PLA | Susp | Abs + PK | 1.59 | 1.73 | | | 0.07 | 0.15 | | |
| BB: dd- | Free/ | Disso + Trans + | 5.19 | | | | 1 | | | |
| PLA | Susp | Abs + PK | 0.43 | | | | 0.15 | | | |
| BB: ga- | Free/ | Disso + Trans + | 25.5 | 20.1 | | | 0.21 | 0.79 | | |
| PLGA | Susp | Abs + PK | 2.87 | 2.95 | | | 0.04 | 0.15 | | |
| BB: dd- | Free/ | Disso + Trans + | 4.51 | | | | 1 | | | |
| PLGA | Susp | Abs + PK | 0.43 | | | | 0.16 | | | |
| BB | Protamine | Disso + Trans + | 17.5 | | 12.1 | | 0.31 | | 0.69 | |
| Alone | Complex | Abs + PK | 1.16 | | 1.34 | | 0.04 | | 0.09 | |
| BB: la- | Protamine | Disso + Trans + | 29.9 | | | 25.0 | 0.18 | 0.25 | 0.41 | 0.16 |
| PLA | Complex | Abs + PK | 5.80 | | | 2.08 | 0.04 | 0.08 | 0.09 | 0.21 |
| BB: dd- | Protamine | Disso + Trans + | 35.6 | | | 17.5 | 0.15 | | 0.34 | 0.51 |
| PLA | Complex | Abs + PK | 4.56 | | | 1.16 | 0.03 | | 0.06 | 0.15 |
| BB: ga- | Protamine | Disso + Trans + | 112 | | | 37.7 | 0.05 | 0.18 | 0.11 | 0.66 |
| PLGA | Complex | Abs + PK | 35.7 | | | 3.17 | 0.02 | 0.06 | 0.04 | 0.38 |
| BB: dd- | Protamine | Disso + Trans + | 30.0 | | | 17.5 | 0.18 | | 0.40 | 0.42 |
| PLGA | Complex | Abs + PK | 3.27 | | | 1.16 | 0.03 | | 0.06 | 0.12 |

As shown in Table 13, the additive model does not generally predict the observed MRTs. Accordingly, there appears to have been some interaction (synergy) between polymer and protein complex which contributes to MRT. The fractional contribution of this interaction is listed in the last column of the table.

In summary, clear differences were observed between acid end group polymers (e.g., acid-initiated polymers) and ester-end group polymers (e.g., dodecanol-initiated) polymers in the delivery of free rhGH suspended in BB:polymer vehicles. The addition of dodecanol-initiated polymers provided no more control of rhGH delivery than did BB alone. This was the case for polymers having M_{w}˜6.5-14 kDa, and for both PLA and 65:35 PLGA (65:35 refers to the respective fractions or percents of lactide and glycolide residues in the polymer). rhGH release from suspensions of rhGH:protamine complex in BB alone was extended relative to suspension of free protein. The protamine complex and polymer apparently worked synergistically to control protein release (extend MRT), and this synergy accounted for 40-70% of the observed MRT.

### Example 12

### Further In-Vivo Depot Characterization

Two additional rhGH complexes were tested in vehicles containing either Lactate-initiated PLA, M_{w}=15.1 kDa, or Dodecanol-initiated PLA, M_{w}=13.9 kDa and compared with un-complexed (free) rhGH formulations. The formulations and sampling times were as described generally in Table 14.

TABLE 14

| | | | |
|---|---|---|---|
| Free rhGH | BB alone | Formulation 1 | blood samples |
| | | | at −24, 0.5, 1, 2, |
| | | | 4, 8, 12, 24 h, 2, |
| | | | 3, 5 d |
| | BB:la-PLA | Formulation 2 | blood samples |
| | 80:20 | | at −24, 0.5, 1, 2, |
| | (%, w/w) | | 4, 8, 12, 24 h, |
| | | | 2, 3, 5 d |
| | BB:dd-PLA | Formulation 3 | blood samples |
| | 80:20 | | at −24, 0.5, 1, 2, |
| | (%, w/w) | | 4, 8, 12, 24 h, |
| | | | 2, 3, 5 d |
| rhGH:Zn²⁺ 1:10 | BB alone | Formulation 4 | blood samples |
| (m/m) | | | at −24, 0.5, 1, 2, |
| | | | 4, 8, 12, 24 h, |
| | | | 2, 3, 5 d |
| | BB:la-PLA | Formulation 5 | blood samples |
| | 80:20 | | at −24, 1, 4, |
| | | | 8, 12, 24 h, 2, 3, |
| | | | 5, 7, 9, 12 d |
| | BB:dd-PLA | Formulation 6 | blood samples |
| | 80:20 | | at −24, 1, 4, |
| | | | 8, 12, 24 h, 2, 3, |
| | | | 5, 7, 9, 12 d |
| rhGH:Zn²⁺:protamine | BB alone | Formulation 7 | blood samples |
| 1:2:0.3 (m/m) | | | at −24, 1, 4, |
| | | | 8, 12, 24 h, 2, 3, |
| | | | 5, 7, 9, 12 d |
| | BB:la-PLA | Formulation 8 | blood samples |
| | 80:20 | | at −24, 1, 4, |
| | | | 8, 12, 24 h, 2, 3, |
| | | | 5, 7, 9, 12 d |
| | BB:dd-PLA | Formulation 9 | blood samples |
| | 80:20 | | at −24, 1, 4, 8, |
| | | | 12, 24 h, 2, 3, 5, |
| | | | 7, 9, 12 d |

### Formulations

Formulation #1; Identity: depot rhGH 1; Description/Physical appearance: Suspension, 50 mg of rhGH in 1 mL of benzyl benzoate (BB); Storage conditions: 2-8° C.

Formulation #2; Identity: depot rhGH 2; Description/Physical appearance: Suspension, LA-PLA, 50 mg of rhGH in 1 mL of BB:PLA₁ (80:20% w/w); Storage conditions: 2-8° C.

Formulation #3; Identity: depot rhGH 3; Description/Physical appearance: Suspension, DD-PLA, 50 mg of rhGH in 1 mL of BB:PLA₂ (80:20% w/w); Storage conditions: 2-8° C.

Formulation #4; Identity: depot rhGH 4; Description/Physical appearance: Suspension, 50 mg of rhGH as Zn²⁺ complex with sucrose, Polysorbate 80 and methionine in 1 mL of BB; Storage conditions: 2-8° C.

Formulation #5; Identity: depot rhGH 5; Description/Physical appearance: Suspension, 50 mg of rhGH as Zn²⁺ complex with sucrose, Polysorbate 80 and methionine in 1 mL of BB:PLA₁ (80:20% w/w); Storage conditions: 2-8° C.

Formulation #6; Identity: depot rhGH 6; Description/Physical appearance: Suspension, DD-PLA, 50 mg of rhGH as Zn²⁺ complex with sucrose, Polysorbate 80 and methionine in 1 mL of BB:PLA₂ (80:20% w/w); Storage conditions: 2-8° C.

Formulation #7; Identity: depot rhGH 7; Description/Physical appearance: Suspension, 50 mg of rhGH as Zn²⁺/protamine complex with sucrose, Polysorbate 80 and methionine in 1 mL of BB; Storage conditions: 2-8° C.

Formulation #8; Identity: depot rhGH 8; Description/Physical appearance: Suspension, LA-PLA, 50 mg of rhGH as Zn²⁺/protamine complex with sucrose, Polysorbate 80 and methionine in 1 mL of BB:PLA₁ (80:20% w/w); Storage conditions: 2-8° C.

Formulation #9; Identity: depot rhGH 9; Description/Physical appearance: Suspension, DD-PLA, 50 mg of rhGH as Zn²⁺/protamine complex with sucrose, Polysorbate 80 and methionine in 1 mL of BB:PLA₂ (80:20% w/w); Storage conditions: 2-8° C.

Abbreviations: BB=Benzyl Benzoate; PLA_{T}=Poly lactic acid (lactate-initiated; M_{w}=15.1 kDa); and PLA₂=Poly lactic acid (dodecanol-initiated; M_{w}=13.9 kDa).

### Dose Preparation and Protocol (Test Articles 1-9)

The vials containing test articles #1-#9 were shaken for about 2 minutes by hand until uniform formulation suspensions were obtained. The flip-off crimps and stoppers were then removed. A 16G, 1½″ needle was placed onto a 1 mL Excel syringe. For test articles #1-9, approximately 1 mL of test article was withdrawn, and 0.1 mL of the test article was back-filled into a 1 mL Terumo Sursaver syringe: 23G ½″ inch pre-attached for test articles; by removing the plunger from back end. The syringe was then primed to deliver for each animal. To avoid needle clogging, the syringe was not primed to 0.1 mL until immediately before administration. The weight of the syringes before and after injection was measured and recorded.

### Results

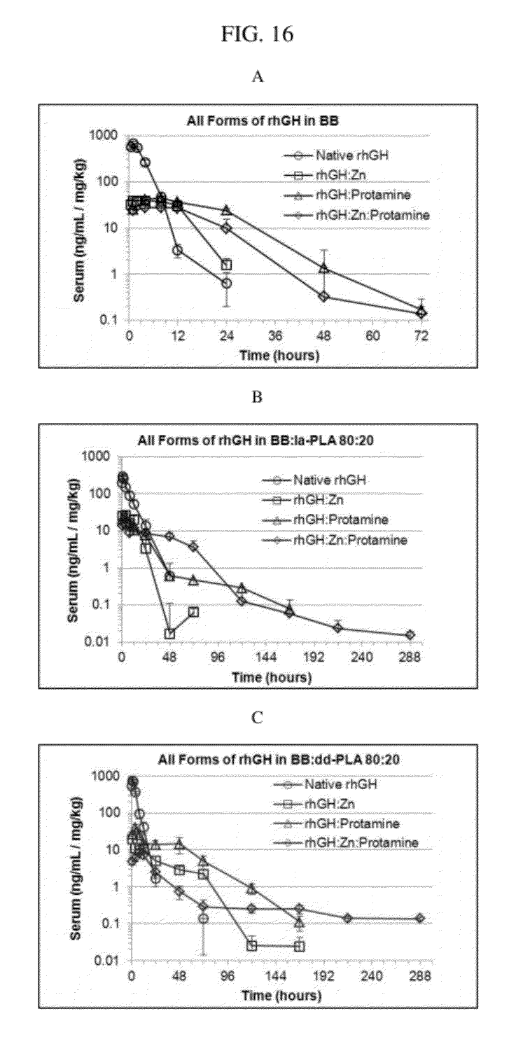

The results of the above experiment are provided in FIG. 16, panels A-C, in which they have been combined with results from Example 11. Plotting dose-normalized serum profiles (ng/mL serum concentration per mg/kg of protein dosed) for each form of rhGH in each vehicle shows that in these formulations complexation reduced serum levels ˜10-fold and extended release independent of polymer content and type. Complexation alone (no polymer, Panel A) extended delivery, with protamine apparently more effective than Zn²⁺, but the combination of the two was no more effective than protamine alone. Addition of la-PLA alone (no complex) also extended delivery, but the effect of dd-PLA alone was equivocal (compare free rhGH in Panels A-C and note that the graphs use different time scales).

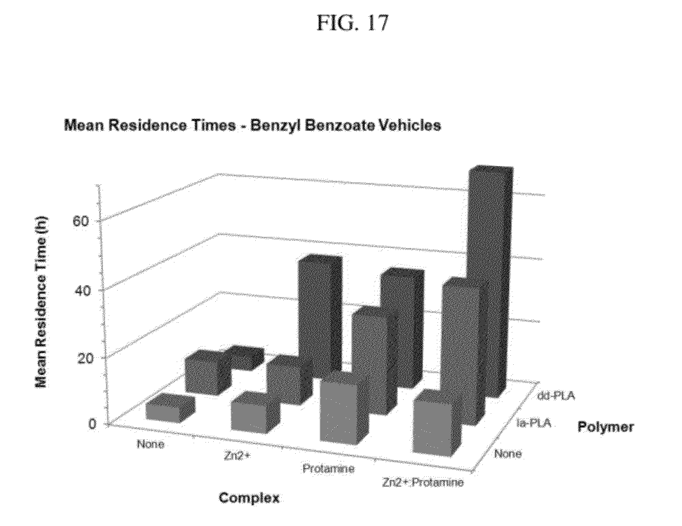

MRTs were calculated for each animal for each formulation and averaged. These results are summarized in FIG. 17. The effects of polymer and complex alone can be discerned by looking along the horizontal axes. Also apparent is the variation in the combined effects of polymer and complex.

As in Example 11, the separate contributions of the complexes and polymers to extending MRT were calculated and an additive model was used to predict MRT for the combined formulations. These results are provided below in Table 15.

TABLE 15

| | | | | | | Additive | | | | |
|---|---|---|---|---|---|---|---|---|---|---|
| | | | | | | Model | Fractional Contribution of | | | |
| | | | MRT & | ΔMRT | ΔMRT | for | Each Component | | | |
| Test | Form of | Contributions | SEM | Polymer | Complex | MRT | BB | | | |
| Article | rhGH | to MRT | (h) | (h) | (h) | (h) | Alone | Polymer | Complex | Synergy |
| BB | Free/ | Disso + Trans + | 2.80 | | | | | | | |
| Alone | Susp | Abs + PK | 0.10 | | | | | | | |
| BB: la- | Free/ | Disso + Trans + | 8.12 | 5.32 | | | 0.34 | 0.66 | | |
| PLA | Susp | Abs + PK | 1.29 | 1.30 | | | 0.06 | 0.19 | | |
| BB: dd- | Free/ | Disso + Trans + | 4.00 | 1.20 | | | 0.70 | 0.30 | | |
| PLA | Susp | Abs + PK | 0.40 | 0.41 | | | 0.07 | 0.11 | | |
| BB | Zn²⁺ | Disso + Trans + | 8.38 | | 5.58 | | 0.33 | | 0.67 | |
| Alone | Complex | Abs + PK | 0.22 | | 0.24 | | 0.02 | | 0.03 | |
| BB: la- | Zn²⁺ | Disso + Trans + | 11.9 | | | 13.7 | 0.24 | 0.45 | 0.47 | |
| PLA | Complex | Abs + PK | 1.77 | | | 1.32 | 0.04 | 0.13 | 0.07 | |
| BB: dd- | Zn²⁺ | Disso + Trans + | 37.7 | | | 9.59 | 0.07 | 0.03 | 0.15 | 0.75 |
| PLA | Complex | Abs + PK | 6.66 | | | 0.47 | 0.01 | 0.01 | 0.03 | 0.22 |
| BB | Zn²⁺/Protamine | Disso + Trans + | 15.1 | | 12.3 | | 0.19 | | 0.81 | |
| Alone | Complex | Abs + PK | 2.15 | | 2.15 | | 0.03 | | 0.18 | |
| BB: la- | Zn²⁺/Protamine | Disso + Trans + | 41.0 | | | 20.4 | 0.07 | 0.13 | 0.30 | 0.50 |
| PLA | Complex | Abs + PK | 5.96 | | | 2.51 | 0.01 | 0.04 | 0.07 | 0.17 |
| BB: dd- | Zn²⁺/Protamine | Disso + Trans + | 69.0 | | | 16.3 | 0.04 | 0.02 | 0.18 | 0.76 |
| PLA | Complex | Abs + PK | 13.2 | | | 2.19 | 0.01 | 0.01 | 0.05 | 0.24 |

Again, the additive model did not adequately predict the observed MRTs (except for the Zn²⁺ complex in la-PLA) indicating that there is a synergistic effect of polymer and complex for some formulations.

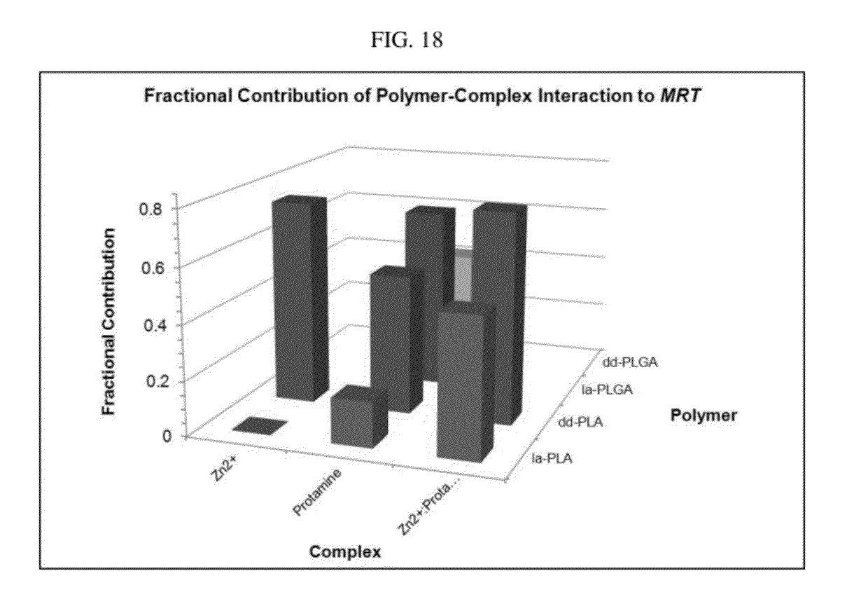

The fractional contributions of BB alone, polymer and complex to MRT were similar across Examples 11 and 12, but the synergistic contributions were somewhat greater in Example 12. The fractional contribution of polymer-complex interaction for Examples 11 and 12 to MRT is provided in FIG. 18. The following combinations were not tested and accordingly the interaction contributions were not determined: la-PLGA:Zn²⁺:protamine; dd-PLGA: Zn²⁺:protamine; la-PLGA:Zn²⁺; and dd-PLGA: Zn²⁺.

In summary, the results of Example 12 corroborate and extend those of Example 11. The effects, individual and synergistic, of the rhGH:protamine complex were also observed with rhGH:Zn²⁺ and the complex formed with both Zn²⁺ and protamine. Without intending to be bound by any particular theory, formulating with a complex of rhGH may afford latitude in the choice of polymer, compensating for intrinsic differences in the capacity of acid- and ester-terminated polymers to control protein release.

### Example 13

### Further In-Vivo Depot Characterization

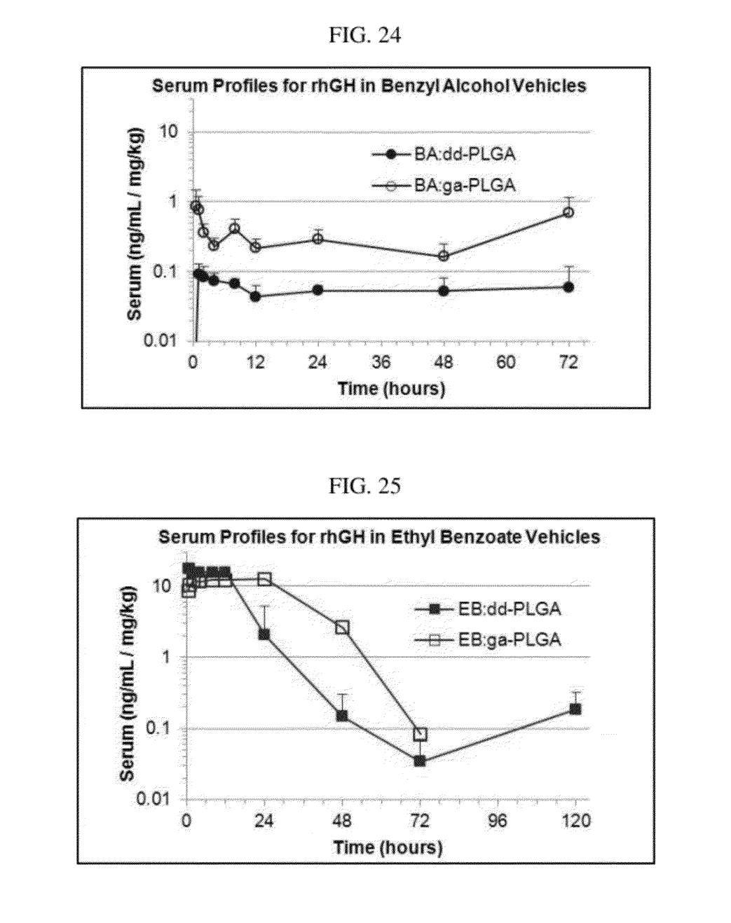

Additional experiments were conducted to determine the suitability of additional solvent-polymer combinations. The tested formulations were as follows: BA:dd-PLGA (6.7 kDa, dodecanol-initiated, 65:35 L:G); BA:ga-PLGA (11.5 kDa, glycolate-initiated, 64:36 L:G); EB:dd-PLGA (ethyl benzoate); EB:ga-PLGA. All vehicles contained 80:20 (% w/w) solvent:polymer ratio. Native rhGH (freeze dried) was used as the beneficial agent (both free and complexed with protamine) except where noted. PK was monitored over a period of 7 days, with samples taken at 0.5, 1, 2, 4, 8, 12, 24, 48, 72, 120 and 168 hours. Group mean dose-normalized serum profiles for the above formulations are provided in FIG. 24 (BA:dd-PLGA and BA:ga-PLGA) and 25 (EB: dd-PLGA and EB: ga-PLGA). All non-zero values are shown.

Unexpectedly, delivery from BA:PLGA vehicles was extremely low, with bioavailability <0.2 and 2%, respectively. Delivery from the EB:PLGA was comparable to what has been shown previously herein for BB:PLGA. Peak serum concentrations for the dd-polymers appeared to be lower, possible due to assay saturation. Differences in MRT between ester- and acid-terminated polymers were less pronounced than in BB-PLGA vehicles. MRT was calculated for each of the above formulations and the results are provided below in Table 16.

TABLE 16

| Test Article | MRT (h) | Test Article | MRT (h) |
|---|---|---|---|
| Free rhGH + | 8.1 | Free rhGH + | 23.4 |
| EB:dd-PLGA, | 9.9 | EB:ga-PLGA, | 16.6 |
| 80:20 | 15.5 | 80:20 | 16.9 |
| | 18.9 | | 18.5 |
| | 12.7 | | 17.4 |
| | 8.0 | | 16.6 |
| | 12.2 | | 18.2 |
| | 1.95 | | 1.18 |
| Test Article | MRT_{inf} (h) | Test Article | MRT_{inf} (h) |
| Free rhGH + | 3.42 | Free rhGH + | 29.2 |
| BB:dd-PLGA | 3.60 | BB:ga-PLGA | 20.8 |
| 80:20 | 4.36 | 80:20 | 20.9 |
| | 5.44 | | 20.3 |
| | 5.82 | | 25.4 |
| | 4.39 | | 36.6 |
| | 4.51 | | 25.5 |
| | 0.43 | | 2.87 |

The duration of rhGH delivery from suspensions of free rhGH in EB:dd-PLGA was >that from comparable BB-based vehicles tested previously herein. The duration of rhGH delivery from suspensions of free rhGH in EB:ga-PLGA was ≦that from comparable BB-based vehicles tested previously herein. The very low rhGH delivery from the BA formulations was unexpected, in light of its structural similarity to EB and BB.

Release of rhGH in vitro from the BA formulations was quite low, <1% over almost 11 days. Moreover, recovery of intact protein from these depots into PBS extraction medium at the end of the release experiment was <1%, but greatly improved by addition of 6N guanidine, suggesting extensive protein aggregation in the formulation. Recovery of rhGH from the EB-based formulations was nearly complete and unaffected by addition of 6N guanidine to the extraction medium.

The observations in vitro and in vivo suggest some specific interaction between BA and rhGH, although formulations of rhGH with 10% BA have performed as well in vivo as formulations containing BB alone. There is also the possibility that delivery of rhGH from the BA:PLGA formulations occurs over much longer times than observed here.

These results may suggest the utility of BA and EB for injectable depots formulations designed for shorter durations of delivery—several days to one week.

### Example 14

### “Cloud” Characterization

As discussed previously herein, it is believed that the beneficial release characteristics of the injectable, biodegradable depot compositions of the present disclosure are due at least in part to the formation of a very fluid, non-structured (without any appreciable mechanical integrity), “rate-controlling cloud” or “rate-controlling film” on the surface of the depot in vivo. The desirable controlled delivery characteristic of the disclosed depot compositions may result from the rate-controlling contributions of both the insoluble beneficial agent complex dispersed in the liquid core of the depot and the polymer cloud or film on the surface of the depot.

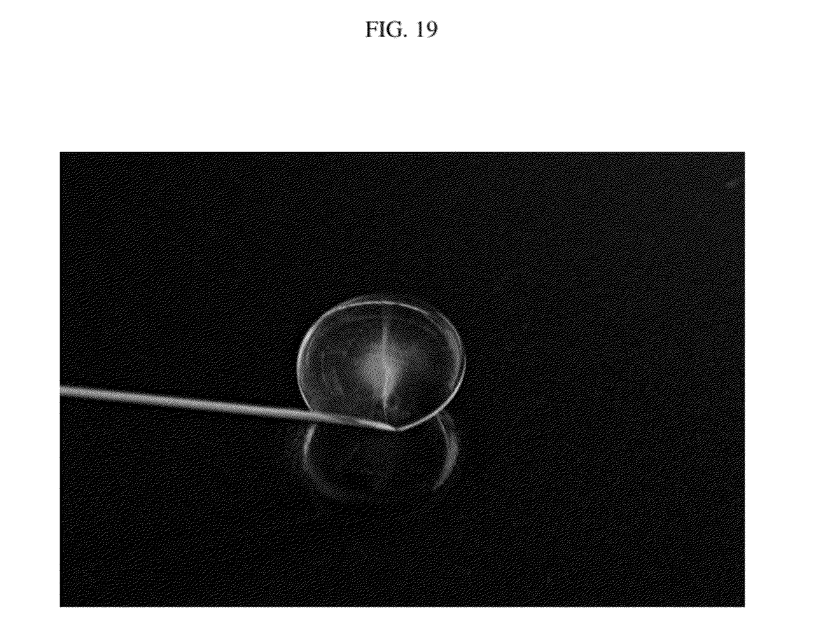

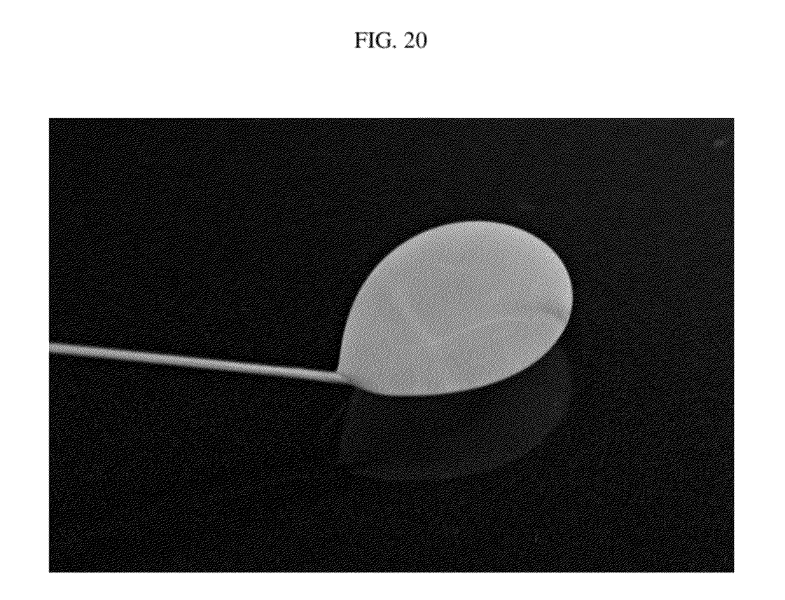

The physical development of this rate controlling cloud can be seen visually in situ as demonstrated in FIGS. 19 and 20. A 23 Gauge regular needle was used to inject approximately 0.5 mL of a SAIB/BB/PLA (LA-initiated) (8:72:20) vehicle into PBS buffer at pH 7.4 and 37° C. A first picture (FIG. 19) was taken at about 10 sec following initiation of injection and a second picture (FIG. 20) was taken about 60 seconds following the completion of the 0.5 mL injection. FIG. 19 shows a slight development of opacity in the center of the vehicle which is likely due to the initial contact of the vehicle with the PBS and is considered an artifact of the procedure. A nearly opaque white cloud is formed over the entire surface of the vehicle by the 60 second time point as shown in FIG. 20.

Cloud formation kinetics are described for a variety of hydrophobic solvent:PLA combinations in Table 17 below, wherein one of index numbers 0-4 is selected based on a visual characterization of the transmittance of the vehicle, where 0 indicates approximately 100% transmittance, 1 indicates greater than approximately 80% transmittance, 2 indicates greater than approximately 50% transmittance, 3 indicates less than approximately 50% transmittance, and 4 indicates approximately 0% transmittance.

### Sample Preparation

Test samples were prepared at three concentration levels of PLA (10%, 20% and 30% w/w) for each solvent by mixing on a rotator until the polymer was completely dissolved.

### Cloud Formation Testing Conditions

The test sample volume was 1 mL and the testing medium was 100 ml of 10 mM PBS at pH 7.4 in French Square Bottles, Wide Mouth, Qorpak® 120 mL (4 OZ) with Fluoropolymer Resin-lined Green Thermoset Cap. The testing temperature was 37° C. For testing, 100 mL of the medium was transferred into the French Square Bottles. The medium was equilibrated in the bottle at 37° C. in an incubator. 1 mL of the polymer solution was pipetted into the bottom corner of the bottles and slowly released. The bottles were then placed back in the incubator at 37° C. At the specified time point the bottles were removed from the incubator and the compositions were visually inspected. The extent of opacity (cloudiness) was recorded using index numbers 1˜4 as defined above and the bottles were placed back in the incubator.

TABLE 17

| | | Time (hr) | | | | | |
|---|---|---|---|---|---|---|---|
| Solvent | PLA w/w % | 0 | 0.5 | 1 | 4 | 6 | 24 |
| Benzyl OH | 10 | 0 | 0 | 0 | 0 | 2 | 3 |
| | 20 | 0 | 2 | 3 | 3 | 3 | 3 |
| | 30 | 0 | 2 | 3 | 3 | 3 | 3 |
| Methyl | 10 | 0 | 2 | 2 | 2 | 2 | 2 |
| Benzoate | 20 | 1 | 4 | 4 | 4 | 4 | 4 |
| | 30 | 1 | 4 | 4 | 4 | 4 | 4 |
| Ethyl | 10 | 1 | 2 | 3 | 3 | 3 | 3 |
| Benzoate | 20 | 1 | 4 | 4 | 4 | 4 | 4 |
| | 30 | 1 | 4 | 4 | 4 | 4 | 4 |
| Propyl | 10 | 0 | 0 | 2 | 2 | 2 | 2 |
| Benzoate | 20 | 0 | 0 | 3 | 3 | 3 | 3 |
| | 30 | 0 | 0 | 3 | 3 | 3 | 3 |
| Butyl | 10 | 0 | 3 | 3 | 4 | 4 | 4 |
| Benzoate | 20 | 1 | 3 | 4 | 4 | 4 | 4 |
| | 30 | 1 | 3 | 4 | 4 | 4 | 4 |
| Benzyl | 10 | 0 | 3 | 3 | 4 | 4 | 4 |
| Benzoate | 20 | 0 | 3 | 4 | 4 | 4 | 4 |
| | 30 | 0 | 3 | 4 | 4 | 4 | 4 |
| Triacetin | 10 | 0 | 1 | 3 | 4 | 4 | 4 |
| | 20 | 1 | 1 | 4 | 4 | 4 | 4 |
| | 30 | 1 | 1 | 4 | 4 | 4 | 4 |
| Triethyl | 10 | 0 | 1 | 2 | 2 | 2 | 2 |
| Citrate | 20 | 0 | 1 | 3 | 3 | 3 | 3 |
| | 30 | 0 | 1 | 3 | 3 | 3 | 3 |

As shown in the above table, significant cloud formation as evidenced by reduced transmittance occurred in each of the above vehicles (with the exception of the benzyl alcohol-10% PLA vehicle) by the 1 hour time point.

### Example 15

### Further “Cloud” Characterization

The rate-controlling, cloud forming vehicles of the present disclosure can also be characterized by their lack of gel-forming characteristics when aged at 37° C. This can be demonstrated by monitoring viscosity stability over time at the selected temperatures. Vehicle compositions were prepared as indicated in Table 18 below.

TABLE 18

| Compositions | Polymer Type |
|---|---|
| SAIB/BA/PLA (8/72/20) | 15.2 KD, lactate |
| | initiated |
| SAIB/BB/BA/PLA | 15.2 KD, lactate |
| (20/60/10/10) | initiated |
| SAIB/BB/EtOH/PLGA | 6.7 KD, dodecanol |
| 65:35 (8/67/5/20) | initiated |
| BB/BA/PLA (70/10/20) | 15.2 KD, lactate |
| | initiated |

The 4 vehicles were placed in glass vials and incubated at 37° C. for 14 days. Dynamic viscosity was measured using an Anton Paar MCR301 rheometer at constant strain of 10% and an angular frequency range of 0.1-100 s⁻¹ at 25° C. The other test conditions were: Quantity of test material: 100 μl and the gap distance between the stationary and rotating conical plate: 0.05 mm.

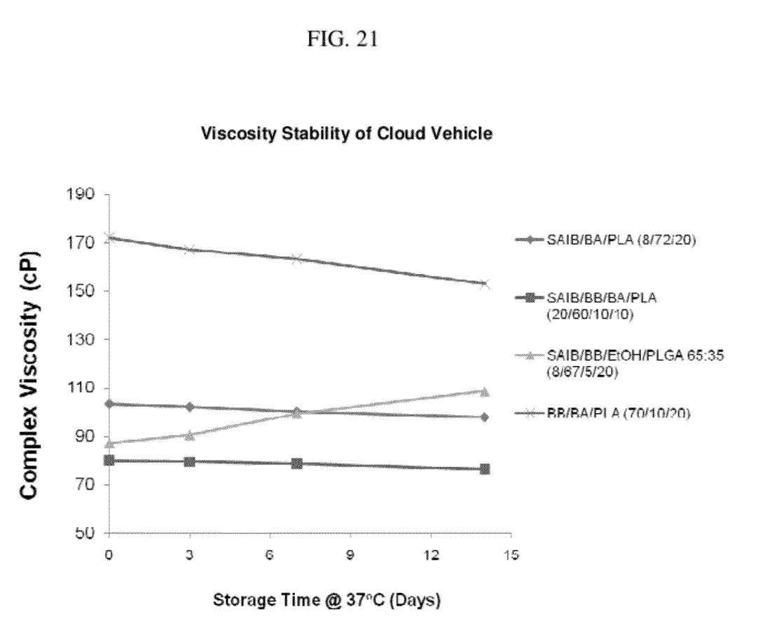

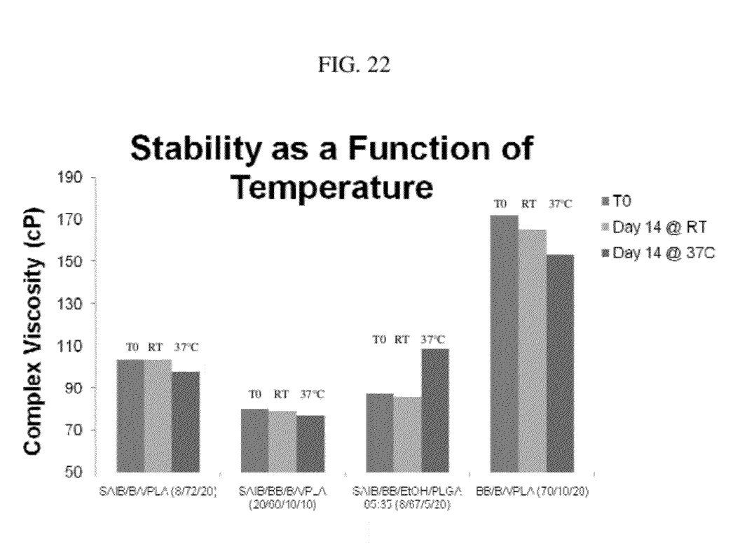

The results for the vehicles aged at 37° C. are shown in FIG. 21. Stability as a function of temperature is shown in FIG. 22. Viscosity measurements for days 3, 7 and 14 are provided below in Table 19.

TABLE 19

| | Complex Viscosity (cP) | | | | |
|---|---|---|---|---|---|
| | | Day | Day | Day | Day |
| | | 3² @ | 7² @ | 14² @ | 14² @ |
| Compositions | T0¹ | 37° C. | 37° C. | 37° C. | RT |
| SAIB/BA/PLA | 104 ± 0.7 | 102 | 100 | 98 | 103 |
| (8/72/20) | | | | | |
| SAIB/BB/BA/PLA | 80 ± 0.1 | 80 | 79 | 76 | 79 |
| (20/60/10/10) | | | | | |
| SAIB/BB/EtOH/PLGA | 87 ± 1.7 | 91 | 100 | 109 | 86 |
| 65:35 (8/67/5/20) | | | | | |
| BB/BA/PLA | 172 ± 2.0 | 167 | 163 | 153 | 165 |
| (70/10/20) | | | | | |

¹mean ± standard deviation of n = 3
²mean of n = 2

Values for G′ (storage modulus) and G″ (loss modulus) were determined and the damping factor Tan 6 (G″IG′) was calculated. These results are shown below in Tables 20-27.

TABLE 20
#1: SAIB/BA/PLA (8/72/20)

| Angular | | | | | | | | |
|---|---|---|---|---|---|---|---|---|
| Frequency | T0 | | Day 3 | | Day 7 | | Day 14 | |
| (s − 1) | G′ (Pa) | G″ (Pa) | G′ (Pa) | G″ (Pa) | G′ (Pa) | G″ (Pa) | G′ (Pa) | G″ (Pa) |
| 1 | 2.22E−05 | 0.111 | 2.18E−05 | 0.109 | 2.08E−05 | 0.104 | 2.21E−05 | 0.110 |
| 1.58 | 3.22E−05 | 0.161 | 3.26E−05 | 0.163 | 3.16E−05 | 0.158 | 3.26E−05 | 0.163 |
| 6.31 | 1.28E−04 | 0.640 | 1.27E−04 | 0.637 | 1.24E−04 | 0.620 | 1.22E−04 | 0.612 |
| 10 | 2.03E−04 | 1.010 | 2.02E−04 | 1.010 | 1.97E−04 | 0.984 | 1.94E−04 | 0.969 |
| 15.8 | 3.23E−04 | 1.610 | 3.21E−04 | 1.610 | 3.13E−04 | 1.570 | 3.07E−04 | 1.530 |
| 25.1 | 5.10E−04 | 2.550 | 5.07E−04 | 2.540 | 4.95E−04 | 2.480 | 4.84E−04 | 2.420 |
| 39.8 | 8.17E−04 | 4.090 | 8.14E−04 | 4.070 | 7.95E−04 | 3.980 | 7.75E−04 | 3.880 |
| 63.1 | 1.31E−03 | 6.570 | 1.31E−03 | 6.540 | 1.29E−03 | 6.430 | 1.25E−03 | 6.230 |
| 100 | 2.14E−03 | 10.700 | 2.17E−03 | 10.900 | 2.13E−03 | 10.600 | 2.04E−03 | 10.200 |

TABLE 21
#2: SAIB/BB/BA/PLA (20/60/10/10)

| Angular | | | | | | | | |
|---|---|---|---|---|---|---|---|---|
| Frequency | T0 | | Day 3 | | Day 7 | | Day 14 | |
| (s − 1) | G′ (Pa) | G″ (Pa) | G′ (Pa) | G″ (Pa) | G′ (Pa) | G″ (Pa) | G′ (Pa) | G″ (Pa) |
| 1 | 1.81E−05 | 0.091 | 1.71E−05 | 0.086 | 1.54E−05 | 0.077 | 1.69E−05 | 0.084 |
| 1.58 | 2.49E−05 | 0.124 | 2.51E−05 | 0.125 | 2.43E−05 | 0.122 | 2.43E−05 | 0.121 |
| 6.31 | 9.84E−05 | 0.492 | 9.78E−05 | 0.489 | 9.71E−05 | 0.485 | 9.48E−05 | 0.474 |
| 10 | 1.55E−04 | 0.777 | 1.56E−04 | 0.778 | 1.54E−04 | 0.770 | 1.51E−04 | 0.753 |
| 15.8 | 2.48E−04 | 1.240 | 2.48E−04 | 1.240 | 2.45E−04 | 1.230 | 2.40E−04 | 1.200 |
| 25.1 | 3.92E−04 | 1.960 | 3.92E−04 | 1.960 | 3.88E−04 | 1.940 | 3.78E−04 | 1.890 |
| 39.8 | 6.29E−04 | 3.150 | 6.31E−04 | 3.160 | 6.25E−04 | 3.120 | 6.07E−04 | 3.030 |
| 63.1 | 1.01E−03 | 5.070 | 1.02E−03 | 5.110 | 1.02E−03 | 5.100 | 9.94E−04 | 4.970 |
| 100 | 1.69E−03 | 8.450 | 1.72E−03 | 8.600 | 1.72E−03 | 8.590 | 1.64E−03 | 8.190 |

TABLE 22
#3: SAIB/BB/EtOH/PLGA 65:35 (8/67/5/20)

| Angular | | | | | | | | |
|---|---|---|---|---|---|---|---|---|
| Frequency | T0 | | Day 3 | | Day 7 | | Day 14 | |
| (s − 1) | G′ (Pa) | G″ (Pa) | G′ (Pa) | G″ (Pa) | G′ (Pa) | G″ (Pa) | G′ (Pa) | G″ (Pa) |
| 1 | 1.72E−05 | 0.086 | 1.94E−05 | 0.097 | 1.97E−05 | 0.098 | 2.21E−05 | 0.111 |
| 1.58 | 1.00E−03 | 0.139 | 2.78E−05 | 0.139 | 3.12E−05 | 0.156 | 3.42E−05 | 0.171 |
| 6.31 | 1.07E−04 | 0.537 | 1.11E−04 | 0.553 | 1.24E−04 | 0.620 | 1.36E−04 | 0.678 |
| 10 | 1.70E−04 | 0.852 | 1.75E−04 | 0.877 | 1.97E−04 | 0.985 | 2.14E−04 | 1.070 |
| 15.8 | 2.71E−04 | 1.350 | 2.82E−04 | 1.410 | 3.14E−04 | 1.570 | 3.40E−04 | 1.700 |
| 25.1 | 4.28E−04 | 2.140 | 4.43E−04 | 2.210 | 4.99E−04 | 2.490 | 5.38E−04 | 2.690 |
| 39.8 | 6.86E−04 | 3.430 | 7.07E−04 | 3.530 | 7.97E−04 | 3.980 | 8.58E−04 | 4.290 |
| 63.1 | 1.10E−03 | 5.520 | 1.15E−03 | 5.770 | 1.30E−03 | 6.490 | 1.38E−03 | 6.910 |
| 100 | 1.85E−03 | 9.230 | 1.93E−03 | 9.640 | 2.14E−03 | 10.700 | 2.26E−03 | 11.300 |

TABLE 23
#4: BB/BA/PLA (70/10/20)

| Angular | | | | | | | | |
|---|---|---|---|---|---|---|---|---|
| Frequency | T0 | | Day 3 | | Day 7 | | Day 14 | |
| (s − 1) | G′ (Pa) | G″ (Pa) | G′ (Pa) | G″ (Pa) | G′ (Pa) | G″ (Pa) | G′ (Pa) | G″ (Pa) |
| 1 | 3.55E−05 | 0.177 | 3.37E−05 | 0.168 | 3.29E−05 | 0.165 | 3.14E−05 | 0.157 |
| 1.58 | 5.54E−05 | 0.277 | 5.24E−05 | 0.262 | 5.16E−05 | 0.258 | 4.87E−05 | 0.243 |
| 6.31 | 2.17E−04 | 1.080 | 2.09E−04 | 1.040 | 2.05E−04 | 1.030 | 1.92E−04 | 0.960 |
| 10 | 3.44E−04 | 1.720 | 3.32E−04 | 1.660 | 3.25E−04 | 1.630 | 3.04E−04 | 1.520 |
| 15.8 | 5.46E−04 | 2.730 | 5.27E−04 | 2.630 | 5.16E−04 | 2.580 | 4.84E−04 | 2.420 |
| 25.1 | 8.63E−04 | 4.320 | 8.33E−04 | 4.170 | 8.18E−04 | 4.090 | 7.65E−04 | 3.820 |
| 39.8 | 1.38E−03 | 6.880 | 1.33E−03 | 6.640 | 1.30E−03 | 6.510 | 1.22E−03 | 6.100 |
| 63.1 | 2.20E−03 | 11.000 | 2.12E−03 | 10.600 | 2.08E−03 | 10.400 | 1.95E−03 | 9.770 |
| 100 | 3.56E−03 | 17.800 | 3.44E−03 | 17.200 | 3.38E−03 | 16.900 | 3.17E−03 | 15.900 |

TABLE 24
#1: SAIB/BA/PLA (8/72/20)

| | |
|---|---|
| Angular | |
| Frequency | Damping Factor (tan δ, G″/G′) |

| (s − 1) | T0 | Day 3 | Day 7 | Day 14 |
|---|---|---|---|---|
| 1 | 5000 | 5000 | 5000 | 4977 |
| 1.58 | 5000 | 5000 | 5000 | 5000 |
| 6.31 | 5000 | 5016 | 5000 | 5016 |
| 10 | 4975 | 5000 | 4995 | 4995 |
| 15.8 | 4985 | 5016 | 5016 | 4984 |
| 25.1 | 5000 | 5010 | 5010 | 5000 |
| 39.8 | 5006 | 5000 | 5006 | 5006 |
| 63.1 | 5015 | 4992 | 4984 | 4984 |
| 100 | 5000 | 5023 | 4977 | 5000 |

TABLE 25
#2: SAIB/BB/BA/PLA (20/60/10/10)

| | |
|---|---|
| Angular | |
| Frequency | Damping Factor (tan δ, G″/G′) |

| (s − 1) | T0 | Day 3 | Day 7 | Day 14 |
|---|---|---|---|---|
| 1 | 5006 | 5000 | 5013 | 4994 |
| 1.58 | 4980 | 4980 | 5021 | 4979 |
| 6.31 | 5000 | 5000 | 4995 | 5000 |
| 10 | 5013 | 4987 | 5000 | 4987 |
| 15.8 | 5000 | 5000 | 5020 | 5000 |
| 25.1 | 5000 | 5000 | 5000 | 5000 |
| 39.8 | 5008 | 5008 | 4992 | 4992 |
| 63.1 | 5020 | 5010 | 5000 | 5000 |
| 100 | 5000 | 5000 | 4994 | 4994 |

TABLE 26
#3: SAIB/BB/EtOH/PLGA 65:35 (8/67/5/20)

| | |
|---|---|
| Angular | |
| Frequency | Damping Factor (tan δ, G″/G′) |

| (s − 1) | T0 | Day 3 | Day 7 | Day 14 |
|---|---|---|---|---|
| 1 | 4994 | 5005 | 4985 | 5023 |
| 1.58 | 139 | 5000 | 5000 | 5000 |
| 6.31 | 5019 | 4982 | 5000 | 4985 |
| 10 | 5012 | 5011 | 5000 | 5000 |
| 15.8 | 4982 | 5000 | 5000 | 5000 |
| 25.1 | 5000 | 4989 | 4990 | 5000 |
| 39.8 | 5000 | 4993 | 4994 | 5000 |
| 63.1 | 5018 | 5017 | 4992 | 5007 |
| 100 | 4989 | 4995 | 5000 | 5000 |

TABLE 27
#4: BB/BA/PLA (70/10/20)

| | |
|---|---|
| Angular | |
| Frequency | Damping Factor (tan δ, G″/G′) |

| (s − 1) | T0 | Day 3 | Day 7 | Day 14 |
|---|---|---|---|---|
| 1 | 4986 | 4985 | 5015 | 5000 |
| 1.58 | 5000 | 5000 | 5000 | 4990 |
| 6.31 | 4977 | 4976 | 5024 | 5000 |
| 10 | 5000 | 5000 | 5015 | 5000 |
| 15.8 | 5000 | 4991 | 5000 | 5000 |
| 25.1 | 5006 | 5006 | 5000 | 4993 |
| 39.8 | 4986 | 4992 | 5008 | 5000 |
| 63.1 | 5000 | 5000 | 5000 | 5010 |
| 100 | 5000 | 5000 | 5000 | 5016 |

In the presence of SAIB, the PLA (15.2 KD) vehicles show moderate viscosity decrease @ 37° C. (2-3 cP/week decrease). Without intending to be bound by any particular theory, this may be the result of slow polymer degradation. The polymer degradation was shown to be significantly increased (3-5 fold increase) for the vehicle without SAIB (shielding effect).

The only vehicle prepared with PLGA 65/35 (6.2 KD), on the other hand, shows viscosity increase overtime (11 cP/week increase). Again, without intending to be bound by any particular theory, this is presumably due to gradual polymer chain rearrangement resulting in enhanced van der walls interaction. There is, however, no indication of gel formation as the elastic (storage) modulus is negligible and does not become dominant. Accordingly, the tested vehicles lack gel-forming characteristics.

### Example 16

### Characterization of Additional Complexation Agents

Additional complexation agents were tested in vitro for their ability to precipitate rhGH. The results of this experiment are provided below in Table 28.

Human growth hormone (purchased from Hospira, Adelaide) was complexed with poly lysine, poly arginine, poly adenylic acid (poly-A), or poly thymine (poly-T) in appropriate ratios (as specified in Table 28) to form suspensions. Supernatant was separated from precipitate (ppt) by centrifugation of the complexed material suspensions. The supernatant solution was analyzed for non-complexed hGH by reverse phase liquid chromatography (RPLC).

TABLE 28
Complexation Capability of hGH with Anionic and Cationic Agents

| | | | Supernatant |
|---|---|---|---|
| | Concentration | | Analysis |
| Complexing Agent | ratio | Observation | (% of hGH) |
| Cationic | | | |
| Poly-Lysine | 1:10 | Cloudy ppt | 9.5 |
| Poly-Arginine | 1:10 | Cloudy ppt | 15.8 |
| Anionic | | | |
| Poly A-10 mer | 1:10 | slightly cloudy ppt | 28.4 |
| Poly A-20 mer | 1:10 | slightly cloudy ppt | 28.6 |
| Poly A-150 mer | 1:10 | very slight ppt | 35.2 |
| Hyaluronic Acid | 1:2 | No ppt | >90 |
| Anionic | | | |
| Poly T-10 mer | 1:10 | Cloudy ppt | 17.7 |
| Poly T-20 mer | 1:10 | Cloudy ppt | 10.3 |
| Poly T-1500 mer | 1:10 | Cloudy ppt | 3 |

As indicated Table 28, each of the listed complexation agents (with the exception of Hyaluronic acid) was capable of at least partially precipitating the rhGH beneficial agent. For the cationic agents, poly-lysine was more effective than poly-arginine at precipitating the rhGH. For the anionic agents tested, Poly thymine was more effective at the 1500mer length than at the 20 or 10mer length, while poly adenosine appeared to be slightly more effective at the 10mer length than at the 150 mer length.

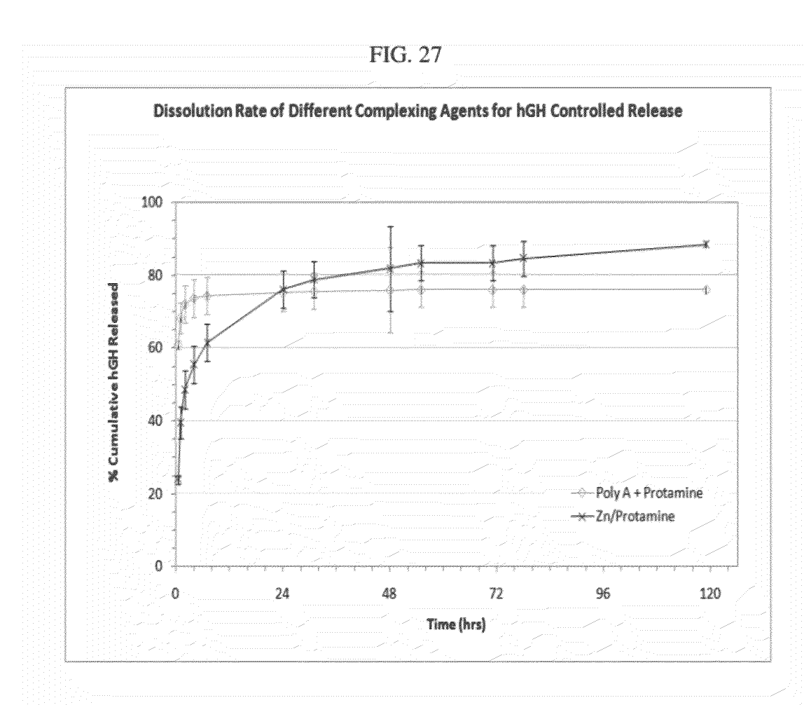

Additional experiments were conducted to characterize the dissolution rates of a hGH beneficial agent complexed with various complexing agents. Solutions of hGH and the different complexing agents were provided in the following ratios to yield an insoluble beneficial agent complex: hGH+Poly-Lysine (1:1), hGH+Poly Adenylic Acid+Protamine (1:0.2:0.3), hGH+Zn+Protamine (1:2:0.3), hGH+Zn (1:10). Free hGH was provided as a control. Dissolution rate was then monitored by reverse phase liquid chromatography (RPLC). The results of these dissolution experiments are provided in FIGS. 26 and 27. Of the above complexes, the Zn/protamine complex provided a more controlled dissolution rate, which will result in a desired release profile.

### Example 17

### Dissolution Rates for Various hGH Complexes

The following powder formulations were prepared and analyzed to determine the effect of various complexing agents on dissolution of hGH in vitro.

Preparation of hGH Powder:

Aliquots of 3 mL each of the bulk hGH solution in buffer from BresaGen were transferred into 5 mL type-Hypak BD glass syringes and lyophilized using the lyophilization cycle provided in Table 1 and a program P90 (optimized for hGH) to fit the steps provided with an FTS lyophilizer, Dura Stop, MP Stoppering Tray Dryer, Stone Ridge, N.Y. Release from this powder was only 40% of the initial hGH content. The balance of the protein denatured or aggregated in the release medium.

Preparation of hGH: Zn Powder:

100 mg of BresaGen hGH powder was placed in a 15 mL wide-mouth glass jar. 5.5 mL of 25 mM NH₄HCO₃ (pH˜7.5) solution was added and the compound was stirred for 30 min at room temperature, 400 rpm until it became clear. Then, 0.45 mL of 100 mM Zinc acetate solution was slowly added to form a white precipitate. The resulting suspension was stirred for 30 min to complete the complexation reaction. 0.19 mL of 290 mM sucrose solution was then added while stirring at 400 rpm. When the solution was clear, 15.2 μL of 10% polysorbate 20 solution was added. Aliquots of 3 mL each of the bulk suspension from the above step were transferred into 5 mL type-Hypak BD glass syringes and lyophilized using the lyophilization cycle provided in Table 1 and a program P90 (optimized for hGH) to fit the steps provided with an FTS lyophilizer, Dura Stop, MP Stoppering Tray Dryer, Stone Ridge, N.Y. From this powder, the released protein is more (>70%) but all the release takes place in less than 48 hrs.

Preparation of hGH:Zn:Protamine Powder:

100 mg of BresaGen hGH powder was placed in a 15 mL wide-mouth glass jar. 5.5 mL of 25 mM NH₄HCO₃ (pH˜7.5) solution was added and the compound was stirred for 30 min at room temperature, 400 rpm until it became clear. Then, 90 uL of 100 mM Zinc acetate solution was added while stirring, followed by 1.02 mL of protamine sulfate solution (conc. 10 mg/mL) was slowly added to form a white precipitate. The resulting suspension was stirred for 30 min to complete the complexation reaction. 0.19 mL of 290 mM sucrose solution was then added while stirring at 400 rpm. When the solution was clear, 15.2 μL of 10% polysorbate 20 solution was added. Aliquots of 3 mL each of the bulk suspension from the above step were transferred into 5 mL type-Hypak BD glass syringes and lyophilized using the lyophilization cycle provided in Table 1 and a program P90 (optimized for hGH) to fit the steps provided with an FTS lyophilizer, Dura Stop, MP Stoppering Tray Dryer, Stone Ridge, N.Y. From this complexed powder of protamine and zinc, the dissolution is slower than either free hGH or Zinc only complex powder.

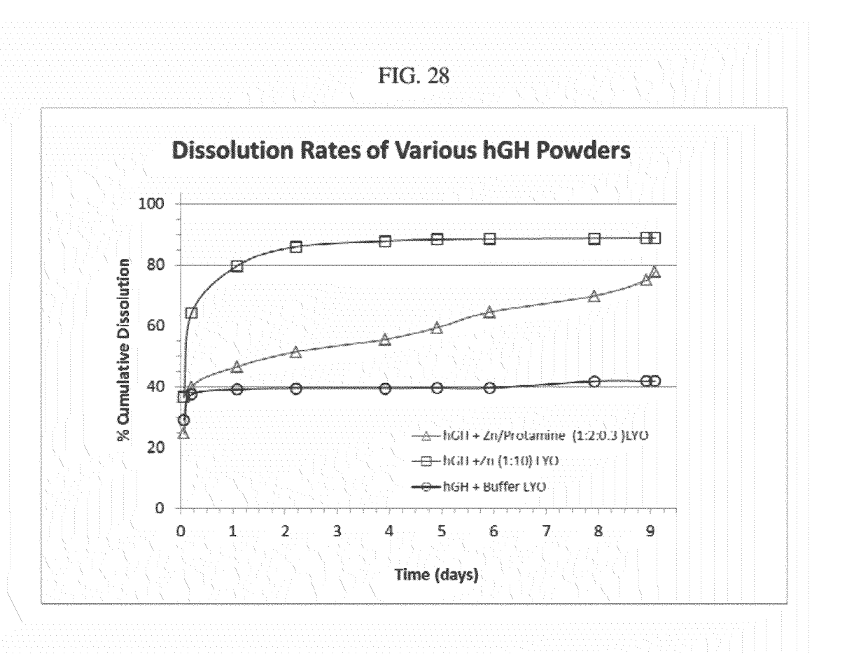

FIG. 28 shows % cumulative dissolution over time for the various preparations.

### Example 18

### Additional Beneficial Agents

Exenatide (purchased from Bachem, Inc.) was complexed with Zinc as Zinc acetate (1:0.4 molar ratio) and with protamine as protamine sulphate (1:0.3) by buffering in to ammonium bicarbonate (50 mM). The resultant suspension containing precipitate was spray-dried using Buchi 329 spray-dryer.

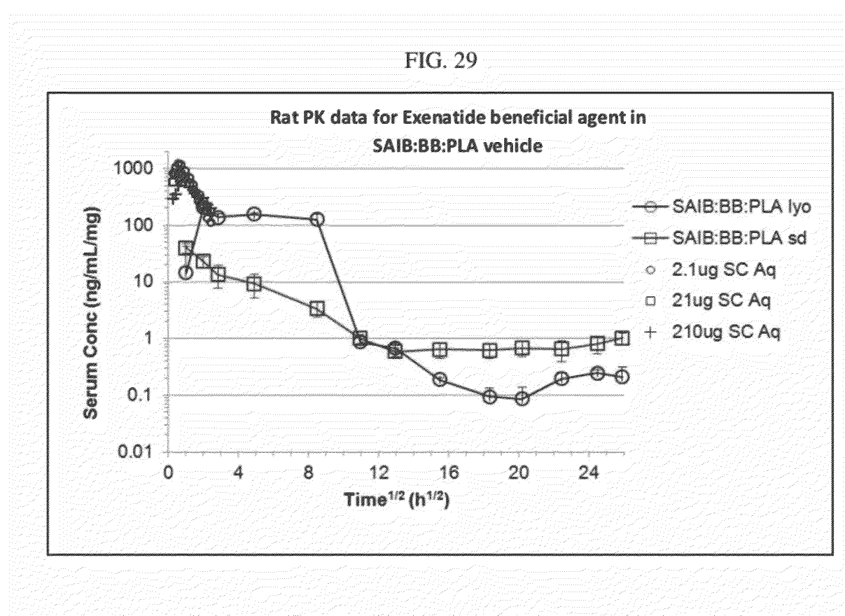

The peptide beneficial agent (Exenatide) was tested in injectable depot compositions according to the present disclosure in order to determine the effect of the depot formulations on release of the beneficial agent in-vivo (rat). The following formulations were tested: Exenatide:protamine 1:2 (m/m), lyophilized, 9.5 mg dose, in SAIB/BB/la-PLA (8/72/20) and Exenatide:protamine 1:2 (m/m), spray dried, 9.5 mg dose, SAIB/BB/la-PLA (8/72/20) methionine & polysorbate 80. These formulations were compared with SC aqueous doses of 2.1 μg, 21 μg and 210 μg. Serum concentration was monitored over time. The results for this experiment are provided in FIG. 29 and demonstrate improved controlled release relative to aqueous bolus.

### Example 19

### Additional Injectability Studies

Additional injectability studies were conducted using a GLP-1 analog as the beneficial agent complexed with protamine or a combination of zinc and protamine and dispersed in a variety of vehicles as described below. Descriptions of the tested formulations are provided below in Table 29 (this GLP-analog is different from the one utilized above in Example 7).

An Instron 3343 instrument was used in the study along with a 1 mL EXEL syringe (EXEL 1 mL Luer Lock Tip Syringe, REF#26050) and a B-D needle in the size of 27G×½″ or 1 mL TERUMO SurSaver Syringe with permanently attached needle 25G×⅝″ (REF#5501D2516). The volume delivered was approximately 0.2 mL and the applied force was 10 lbf. The tests were performed at room temperature of about 21.8° C.-22.2° C. The target peptide content in the formulations was 70 mg/mL. The injectability results for the formulations are provided below.

TABLE 29

| | Complex | | | | | | | | Mean | | |
|---|---|---|---|---|---|---|---|---|---|---|---|
| | Molar Ratio | | | | | | | | Injection | | |
| | to Peptide | | SAIB and Solvents | | | | Polymer | | Time (sec), | | |
| | (as 1) | | (% w/w) | | | | (% w/w) | | n = 3 | | |
| Formulation | Protamine | Zn | SAIB | BB | BA | EtOH | PLA | PLGA | 25G | 27G | Vehicle Composition |
| A | 0.3 | 0.4 | 8 | 72 | 0 | 0 | 20 | 0 | 2.8 | 9.3 | SAIB/BB/PLA 8:72:20 |
| B | 0.3 | 0.4 | 8 | 72 | 0 | 0 | 20 | 0 | 2.3 | 8.6 | SAIB/BB/PLA 8:72:20 |
| C | 0.3 | 0.4 | 20 | 60 | 10 | 0 | 10 | 0 | 1.6 | 2.6 | SAIB/BB/BA/PLA |
| | | | | | | | | | | | 20:60:10:10 |
| D | 0.3 | 0.4 | 0 | 70 | 10 | 0 | 0 | 20 | 1.5 | 2.4 | BB/BA/PLGA |
| | | | | | | | | | | | 70:10:20 |
| E | 0.3 | 0 | 8 | 72 | 0 | 0 | 20 | 0 | 2.7 | 10.9 | SAIB/BB/PLA 8:72:20 |
| F | 0.3 | 0 | 20 | 60 | 10 | 0 | 10 | 0 | 1.6 | 2.9 | SAIB/BB/BA/PLA |
| | | | | | | | | | | | 20:60:10:10 |
| G | 0.3 | 0 | 0 | 70 | 10 | 0 | 20 | 0 | 1.6 | 4.8 | BB/BA/PLA 70:10:20 |
| H | 0.3 | 0.4 | 8 | 67 | 0 | 5 | 0 | 20 | 1.5 | 3.1 | SAIB/BB/EtOH/PLGA |
| | | | | | | | | | | | 8:67:5:20 |

All eight of the above formulations went through needles in the sizes of 25G×⅝″ and 27G×½″ smoothly and were considered to have acceptable injectability.

### Example 20

### Additional Pharmacokinetic Characterization for GLP-1 Analog Formulations

Additional in vivo experiments were performed using the GLP-1 analog formulations described above for Example 19. The sustained release, initial burst and bioavailability characteristics of each of the formulations were determined.

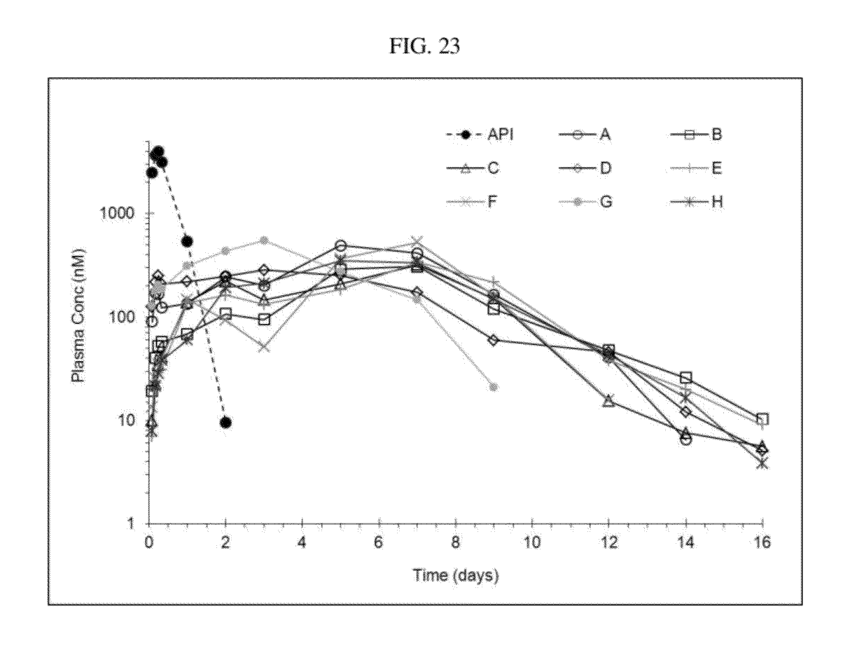

The above formulations were injected subcutaneously into Sprague Dawley rats following removal of the hair at the local injection site. The formulations were administered in a volume of approximately 100 ul with dosages ranging from 7.3 to 9.5 mg/rat with 3 rats per treatment group. These formulations were compared with the administration of API alone at a dose of 2 mg/rat. The average PK profiles for each of the above treatment conditions are shown in FIG. 23.

Based on the above data, it was determined that the drug release rates AUC(Day1)/AUC(Day14) (a measure of initial burst) for each of the above formulations was less than 10%. Some of the formulations (001, 004 and 007) showed a small initial burst at the same time as Tₘₐₓ of the API. The average values for AUC(Day1)/AUC(Day14) are provided below in Table 30.

TABLE 30

| Formulation | AUC_{Day1}/AUC_{Day14} (%) |
|---|---|
| A | 3.9 |
| B | 2.5 |
| C | 3.3 |
| D | 10.0 |
| E | 2.7 |
| F | 3.5 |
| G | 9.2 |
| H | 2.0 |

Bioavailability was calculated based on the above experiments and the results are provided below in Table 31.

TABLE 31

| Formulation | BA Relative to SC API (%) |
|---|---|
| SC API | N/A |
| A | 34 |
| B | 18 |
| C | 22 |
| D | 25 |
| E | 27 |
| F | 27 |
| G | 30 |
| H | 26 |

The above formulations showed acceptable bioavailability of 18-34% relative to the API out to 14 days.

In summary, the above data showed that all of the tested formulations maintained adequate concentration of the GLP-1 analog in rat. In addition, cumulative drug input over 0-24 h (AUC_{day1}/AUC_{day14}) was less than 10% for each of the formulations. The predicted steady state concentrations were entirely within the therapeutic window with the exception of formulations F and G. Finally, each of the above formulations exhibited acceptable bioavailability.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A composition comprising:
a vehicle comprising
a biodegradable polymer present in an amount of from about 5% to about 40% by weight of the vehicle and
a hydrophobic solvent present in an amount of from about 95% to about 60% by weight of the vehicle; and
an insoluble beneficial agent complex dispersed in the vehicle, the insoluble beneficial agent complex having a solubility of less than 1 mg/mL in the vehicle at 25° C.,
wherein the composition has a zero shear viscosity less than 1,200 centipoise at 25° C., and
wherein the composition is not an emulsion.

2. The composition of claim 1, wherein the composition is not a gel.

3. The composition of claim 1, wherein the composition has a G″/G′ ratio of greater than or equal to 10.

4. The composition of claim 1, wherein the biodegradable polymer comprises an ionizable end-group and has a weight average molecular weight ranging from 1000 Daltons to 20,000 Daltons.

5. The composition of claim 1, wherein the biodegradable polymer comprises at least one member selected from poly-lactides, poly-glycolides, poly-caprolactones and copolymers and terpolymers thereof.

6. The composition of claim 1, wherein the biodegradable polymer comprises at least one of polylactic acid and poly(lactic acid-co-glycolic acid).

7. The composition of claim 1, wherein the hydrophobic solvent comprises at least one member selected from benzyl alcohol, methyl benzoate, ethyl benzoate, n-propyl benzoate, isopropyl benzoate, butyl benzoate, isobutyl benzoate, sec-butyl benzoate, tert-butyl benzoate, isoamyl benzoate, and benzyl benzoate.

8. The composition of claim 1, wherein the hydrophobic solvent comprises benzyl benzoate.

9. The composition of claim 1, further comprising benzyl alcohol.

10. The composition of claim 1, further comprising ethanol.

11. The composition of claim 1, wherein the insoluble beneficial agent complex comprises beneficial agent, a divalent metal, and one of a polymeric cationic complexing agent and a polymeric anionic complexing agent.

12. The composition of claim 1, wherein the insoluble beneficial agent complex comprises at least one member selected from protamine, poly-lysine, poly-arginine, polymyxin, carboxy-methyl-cellulose (CMC), poly-adenosine, and poly-thymine.

13. The composition of claim 1, wherein the insoluble beneficial agent complex comprises beneficial agent and protamine.

14. The composition of claim 1, wherein the insoluble beneficial agent complex comprises beneficial agent and a divalent metal or salt thereof.

15. The composition of claim 14, wherein the divalent metal is selected from Zn²⁺, Mg²⁺, and Ca²⁺.

16. The composition of claim 15, wherein the insoluble beneficial agent complex further comprises protamine.

17. The composition of claim 1, wherein the insoluble beneficial agent complex comprises beneficial agent and protamine, wherein the molar ratio of the beneficial agent and protamine is approximately 1:0.1 to 0.5.

18. The composition of claim 1, wherein the insoluble beneficial agent complex comprises beneficial agent, zinc, and protamine, wherein the molar ratio of the beneficial agent, zinc, and protamine is approximately 1:0.4 to 2:0.1 to 0.5.

19. The composition of claim 1, wherein the vehicle consists of a single solvent consisting of the hydrophobic solvent consisting of benzyl benzoate, and the insoluble beneficial agent complex comprises beneficial agent and protamine.

20. The composition of claim 1, wherein the mean residence time (MRT) of beneficial agent in-vivo is greater than the sum of MRTₛₒₗᵥₑₙₜ+ΔMRT_{complex}+ΔMRT_{polymer}, wherein MRTₛₒₗᵥₑₙₜ is the MRT for the beneficial agent in the hydrophobic solvent alone, ΔMRT_{complex} is the change in MRT due to the insoluble beneficial agent complex, in the absence of polymer, and ΔMRT_{polymer} is the change in MRT due to the polymer, in the absence of complexation of the beneficial agent.

21. The composition of claim 1, wherein when 10 mg of the insoluble beneficial agent complex is dispersed and left to stand in 1 mL of a test solution of phosphate buffered saline at pH 7.4 at 37° C. for 24 hours, the amount of beneficial agent dissolved in the test solution is less than 60% of the beneficial agent in the 10 mg of insoluble beneficial agent complex.

22. A composition comprising:
a vehicle comprising
a biodegradable polymer present in an amount of from about 5% to about 40% by weight of the vehicle and
a hydrophobic solvent present in an amount of from about 95% to about 60% by weight of the vehicle; and
an insoluble beneficial agent complex dispersed in the vehicle, the insoluble beneficial agent complex having a solubility of less than 1 mg/mL in the vehicle at 25° C.,
wherein when 0.8 mL of the composition is placed in a 1 mL syringe at 25° C. fitted with a 0.5 inch needle with a gauge of 21 and 10 lbs of force are applied, at least 0.5 mL of the composition is ejected from the syringe in less than 10 seconds, and
wherein the composition is not an emulsion.
